(19) United States
(12) Patent Application Publication
(10) Pub. No.: US 2009/0247455 A1
(43) Pub. Date: Oct. 1, 2009

(54) Isolation of Inhibitors of IRES-Mediated Translation

(76) Inventor: Mark Fear, Western (AU)

Correspondence Address: FENWICK & WEST LLP, SILICON VALLEY CENTER, 801 CALIFORNIA STREET, MOUNTAIN VIEW, CA, 94041, US

(21) Appl. No.: 11/909,785

(22) PCT Filed: Mar. 31, 2006
(86) PCT No.: PCT/AU2006/000435

§ 371 Date: Aug. 27, 2008

(30) Foreign Application Priority Data
Mar. 31, 2005 (AU) 2005901574

(51) Int. Cl. A61K 38/16; C12Q 1/68; C07K 7/08; C07K 7/06; C07K 14/00; A61K 38/10; A61K 38/08; A61K 31/7052; C07K 1/00; A61P 31/12

(52) U.S. Cl. 514/12; 435/6; 530/326; 530/328; 530/324; 530/327; 530/325; 514/13; 514/15; 514/14; 514/44 R; 530/333

(57) ABSTRACT

The present invention relates to a method for identifying or determining a compound that inhibits or reduces internal ribosome entry site (IRES) mediated translation. For example, the present invention provides a method for determining a compound that inhibits IRES-mediated translation, said method comprising expressing in a cell a counter selectable marker operably under the control of an IRES. A candidate compound is then introduced into the cell or contacted with the cell and the cell maintained under conditions that select against a cell expressing the counter-selectable marker gene. Accordingly, a cell in which IRES-mediated translation of the counter-selectable reporter gene is selected, thereby identifying a compound that inhibits IRES-mediated translation. The present invention also provides compounds identified by the method.

### FIELD OF THE INVENTION

The present invention relates to a method for identifying or determining a compound that inhibits or reduces internal ribosome entry site (IRES) mediated translation.

### BACKGROUND OF THE INVENTION

### General

This specification contains nucleotide and amino acid sequence information prepared using PatentIn Version 3.3. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution and solid phase peptide synthesis. Such procedures are described, for example, in the following texts that are incorporated by reference:
(i) Sambrook, Fritsch & Maniatis, Molecular Cloning: A Laboratory Manual, Cold Spring Harbor Laboratories, New York, Second Edition (1989), whole of Vols I, II, and III;
(ii) DNA Cloning: A Practical Approach, Vols. I and II (D. N. Glover, ed., 1985), IRL Press, Oxford, whole of text;
(iii) Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
(iv) Animal Cell Culture: Practical Approach, Third Edition (John R. W. Masters, ed., 2000), ISBN 0199637970, whole of text;
(v) Immobilized Cells and Enzymes: A Practical Approach (1986) IRL Press, Oxford, whole of text;
(vi) Perbal, B., A Practical Guide to Molecular Cloning (1984);
(vii) Methods In Enzymology (S. Colowick and N. Kaplan, eds., Academic Press, Inc.), whole of series;
(viii) J. F. Ramalho Ortigão, “The Chemistry of Peptide Synthesis” In: Knowledge database of Access to Virtual Laboratory website (Interactiva, Germany);
(ix) Sakakibara, D., Teichman, J., Lien, E. Land Fenichel, R. L. (1976). Biochem. Biophys. Res. Commun. 73 336-342
(x) Merrifield, R. B. (1963). J. Am. Chem. Soc. 85, 2149-2154.
(xi) Barany, G. and Merrifield, R. B. (1979) in The Peptides (Gross, E. and Meienhofer, J. eds.), vol. 2, pp. 1-284, Academic Press, New York.
(xii) Bodanszky, M. (1984) Principles of Peptide Synthesis, Springer-Verlag, Heidelberg.
(xiii) Bodanszky, M. & Bodanszky, A. (1984) The Practice of Peptide Synthesis, Springer-Verlag, Heidelberg.

### DESCRIPTION OF THE RELATED ART

In eukaryotic cells translation of mRNA is generally initiated by a mechanism known as Cap-dependent initiation of translation. The majority of mRNAs in eukaryotes comprise a mGpppX cap structure at their 5′ end. This cap interacts with the eIF4E subunit of the heterotrimeric translation initiation factor eIF4F. This complex of proteins facilitates docking of the ribosomal 43S subunit which then scans the mRNA from the 5′ untranslated region until an initiation codon is detected. The ribosomal 60S subunit then binds to the complex of proteins and translation of the encoded protein commences.

However, a number of mRNAs are transcribed using a mechanism of translation that is Cap-independent. Rather than a 5′ Cap, these mRNAs commonly comprise a large 5′ untranslated region that is highly structured and comprise a region known as an internal ribosome entry site (IRES).

### Internal Ribosome Entry Site

Cap-independent initiation of translation was initially noted in mRNA in poliovirus infected cells (Pelletier and Sonenburg Nature 334: 320-325, 1988). The uncapped 5′ region of the genome of poliovirus (and other picornaviruses) contains a 5′ untranslated region comprising 400 to 500 nucleotides that form dense and stable secondary structures. This highly structured nucleic acid is bound by members of the complex of proteins required for translation at a site adjacent to or directly at an initiation codon used to translate the viral holoprotein.

Subsequent studies using a picornavirus IRES demonstrated that this nucleic acid induces ribosome entry to a downstream open reading frame of a mulitcistronic mRNA (i.e., internal ribosome entry) (Jang et al., J. Virol., 63: 1651-1660, 1989). Furthermore, incorporation of a picornavirus IRES into a circular mRNA facilitates ribosome entry and translation of the circular cistron (Chen and Sarnow, Science 268: 415-417, 1995). Based on these studies, it was concluded that an IRES is a genetic element that facilitates internal ribosome entry and mRNA translation independent of the presence of a cap structure.

Following from these studies, IRESs have been identified in a number of different virus types, including, for example, RNA virus (e.g., bovine diarrhea virus, swine fever virus or hepaciviruses, for example, hepatitis C virus), retrovirus (moloney murine leukemia virus, simian immunodeficiency virus or human immunodeficiency virus) or DNA virus (e.g., human herpesvirus 8). Furthermore, several endogenous eukaryotic mRNAs have also been shown to contain an active IRES (for example, c-myc, BiP, vascular endothelial growth factor (VEGF) or X-linked inhibitor of apoptosis protein (XiaP)).

Analysis of IRES from a number of different viruses indicates that whilst the nucleotide sequence of an IRES may be conserved between strains of the same species, IRESs are highly divergent in their primary structure between different species. Notwithstanding the apparent lack of nucleotide sequence identity, much of the nucleotide sequence variation observed in related organisms merely causes compensatory changes in the secondary structure of an IRES (Martinez-Salas et al., J. Gen Virol., 82: 973-984, 2001). In fact, the secondary and/or tertiary structure of IRESs, particularly, those from related organisms are highly conserved.

To determine those secondary structures required for IRES initiated translation several groups have determined regions of high structural conservation and performed mutagenesis studies. Results from these studies indicate that whilst single base pair changes or small deletions or insertions into the sequence of an IRES reduce the level of IRES-induced translation, it is the overall RNA secondary structure of this region that is required for translational activity (Martinez-Salas et al., J. Gen Virol., 82: 973-984, 2001).

Furthermore, the tertiary structure of an IRES contributes its ability to interact with cellular proteins and initiate Cap-independent translation. For example, the IRES of hepatitis C virus forms a pseudoknot structure and destabilization of this tertiary structure dramatically reduces the level of translation induced by the IRES (Wang et al., RNA, 1: 536-537, 1995).

Using negative stain transmission electron microscopy (TEM), Beales et al., J. Virol., 77: 6574-65579, 2003 showed a high level of conservation in the tertiary structure of IRESs of related viruses. For example, flaviviruses were shown to comprise an IRES that is forked in structure with two long stems variably linked at their bases by a pseudoknot domain. Picornavirus' IRES form a F-shaped structure with a large backbone and two side stems. Based on these results the authors concluded that the high level of structural conservation indicates that IRES tertiary structure is important in inducing Cap-independent translation.

### IRES and Disease

Viral Disease

IRES-mediated translation occurs in a number of diseases and disorders. For example, a number of retroviruses, e.g., picornaviruses, use Cap-independent translation to induce translation of their RNA genome. These viruses also encode a protease that is capable of cleaving eIF4G and rendering this protein incapable of interacting with the Cap-binding protein, EIF4E. Accordingly, picornaviruses suppress Cap-dependent expression of host cell proteins, thereby permitting translation of their own genome without competition from cellular mRNAs.

Viruses that are known to make use of IRES-mediated translation are known to cause a growing number of diseases including, for example, HIV/AIDS, hepatitis C, hepatitis A, foot and mouth disease, bovine viral diarrhea and classic swine fever. Those diseases that are caused by viruses that utilize an IRES affect large populations of individuals, and a number of these diseases are currently incurable.

For example, hepatitis C virus (HCV) infection is one of the most common blood-borne disease in the United States with the Center for Disease Control (CDC) estimating that there are approximately 3.9 million people currently infected in the US alone (i.e. 1.8% of the US population). Treatment for HCV infection and its associated complications (e.g., liver failure or cirrhosis) is estimated to be approximately USD600 million per year with a lifetime cost for a patient that does not undergo a liver transplant being approximately USD100,000.

Another common disease caused by a retrovirus is autoimmune deficiency syndrome (AIDS), caused by the human immunodeficiency virus (HIV) (Barre-Sinossi, F. et al., Science 220:868-870, 1983). HIV exhibits high genetic variation, which results in a variety of biological phenotypes displayed by various strains of the virus and also by the same strain of the virus in a single patient at different times. Phenotypic heterogeneity is found in replication kinetics, susceptibility to serum neutralization, anti-viral drug resistance, induction of cytopathicity and host-cell range specificity. The CDC estimates that as at the end of 2003 there were about 405,000 people living with AIDS in the USA and approximately 1 million people infected with HIV.

Foot and mouth disease is among the most virulent and contagious diseases of farm animals, particularly, economically important species, such as, for example, cattle, sheep and pigs. The disease is endemic in several areas of the world and can be found in many countries of Africa, Asia and South America where it is controlled to varying degrees by immunisation programmes.

Many viruses that make use of an IRES for translation of their genome or a region thereof, particularly, RNA viruses also have a high mutation rate. For example, in HIV approximately 50 percent of transcripts comprise at least one mutation and in HCV the mutation rate is estimated at between 1.44×10⁻³ and 1.92×10⁻³ base substitutions per site per year; Otaga et al., Proc. Natl. Acad. Sci. USA 88: 3392-3396, 1991. This high level of mutation provides a virus with the ability to rapidly become resistant to current therapeutic strategies (Condra et al., J. Virol., 708270-8276, 1990). As a consequence, the high mutation rate is thought to contribute significantly to the high rate of failure of current therapeutic strategies.

Cancer

Several, endogenous genes have also been shown to comprise an IRES. Unlike IRES from viruses, these sequences do not appear to be constitutively active. Rather, translation from a cellular IRES appears to be induced by a specific stimulus, such as, for example, a stage of development, inflammation, angiogenesis or γ-irradiation. As cellular IRES activity appears to be induced by cellular stress it has been postulated that cancer cells escape cell death (e.g., induced by chemotherapy or radiation therapy) or induce cell proliferation by inducing expression of specific genes (e.g., a cell survival gene) using this mechanism (Holccik, Curr. Cancer Drug Targets, 4: 299-311, 2004).

For example, Fraser et al., Reprod Biol. Endocrinol,. 1:1-13, 2003 showed the gene encoding X-linked inhibitor of apoptosis protein (Xiap) comprises an IRES that induces translation of Xiap mRNA by a Cap-independent mechanism during cellular stress, e.g., during chemotherapy and/or radiotherapy. However, proteins that suppress Xiap activity are not translated by a Cap-independent mechanism. As protein translation mechanisms are often suppressed during as a result of chemotherapy, Xiap activity is increased during treatment as a result of suppression of Xiap inhibitor expression. Accordingly, IRES-mediated translation of Xiap induces chemoresistance in cancer cells, particularly, ovarian cancer cells.

Galy et al., Cancer Res. 59: 165-171, 2003, showed that translation of fibroblast growth factor 2 (FGF2) in cancer cells was induced by two IRESs. This leads to increased expression of high molecular weight FGF2, which acts as an intracrine factor that induces cellular proliferation and transformation. In contrast, normal cells translate FGF2 mRNA in a Cap-dependent manner and expression of this gene is reduced in a cell density-dependent manner. Accordingly, FGF2 expression and the associated cell proliferation is reduced as cell density is increased. On the basis of these results the authors conclude that uncontrolled IRES-mediated translation of FGF2 causes continuing cell proliferation despite increased cell density and is associated with cellular transformation.

It is clear from the preceding discussion that IRES-mediated translation plays a role in a number of important diseases that affect both humans and non-human animals. Accordingly, a compound that inhibits IRES-mediated translation is a likely candidate therapeutic for the treatment of a disease associated with IRES-mediated translation, e.g., a viral disease or a cancer.

### Methods for Determining an IRES Inhibitor

Currently, there are few methods for determining an IRES inhibitory compound. One such method (described, for example, in Tallet-Lopez et al., 31: 734-742, 2003) uses a bicistronic vector in which a detectable reporter gene (for example, luciferase) is placed in operable connection with the IRES being studied. The reporter gene from the vector is transcribed, contacted with a test compound and an in vitro translation system, e.g. a rabbit reticulocyte extract that is capable of translating the mRNA. A compound that reduces the level of reporter gene expression relative to a control sample is considered to reduce IRES-mediated translation. The disadvantage of this system is that only some IRESs are amenable to translation using an in vitro translation system. Rather IRES-mediated translation is often restricted to specific cell-types. The method is also both laborious and expensive, requiring the detection of reporter gene expression in a large number of individual assays to identify an inhibitory compound.

Nulf and Corey, Nucl. Acids Res. 32: 3792-3798, 2004 describe a similar method in which the bicistronic vector is introduced into a cell and the level of IRES-mediated translation of a detectable marker determined in the presence or absence of a candidate compound. However, this method requires directly assaying each cell sample into which a test compound is introduced to determine the level of reporter gene expression, and, as a consequence, is both time consuming and expensive.

An alternative assay is described in USSN20030124550 in which a compound is assayed for its ability to inhibit the interaction between the hepatitis C IRES and a 40S ribosomal protein S5. The assay uses a gel shift assay to determine the binding of the IRES to the 40S ribosomal protein S5 in the presence or absence of a test compound. Clearly, this method is both time consuming and laborious, requiring complex molecular techniques to determine the binding of the IRES and the ribosomal subunit. Furthermore, this assay provides no evidence that the test compound is capable of inhibiting IRES-mediated translation, let alone in the context of a cell.

Accordingly, it is clear that there is a need in the art for a rapid and relatively inexpensive method for determining a compound that reduces or inhibits IRES-mediated translation. Such a method is useful, for example, for identifying a candidate antiviral compound and/or a candidate anti-cancer agent.

### SUMMARY OF INVENTION

In work leading up to the present invention the present inventors sought to produce a method that permitted rapid identification of a compound that reduces or inhibits IRES-mediated translation. As exemplified herein, the present inventors have developed an assay that comprises expressing a counter-selectable marker gene, preferably, a positive-negative selectable marker gene operably linked to an IRES in a cell. A cell expressing the reporter gene, i.e., in which the IRES is active is then selected by positive selection. Following introduction of a candidate compound into the cell, negative selection is used to select against a cell still capable of expressing the selectable marker gene. Accordingly, only a cell comprising or expressing a compound capable of inhibiting IRES-mediated translation is capable of growing under negative selection. Accordingly, the present inventors have developed a high-throughput method for the determination of compounds that inhibit or reduce IRES-mediated translation. Using this method, the inventors have also identified a number of peptides capable of reducing or inhibiting IRES-mediated translation.

As a large number of IRESs studied to date are capable of initiating translation of a mRNA in a cell, the present inventors developed an assay using the HCV IRES as a model for IRES-mediated translation generally.

Accordingly, the present invention provides a method for identifying a compound that reduces or inhibits internal ribosome entry site (IRES)-mediated translation, said method comprising:
(i) expressing in a cell a counter-selectable reporter gene operably linked to an IRES;
(ii) contacting the cell with or introducing into the cell a candidate compound under conditions sufficient to kill or inhibit or reduce the growth of a cell expressing the counter-selectable reporter gene; and
(iii) selecting a cell in which the expression of the counter-selectable reporter gene is reduced or inhibited wherein said reduced or inhibited expression is indicative of reduced or inhibited IRES-mediated translation,
thereby identifying a compound that reduces or inhibits IRES-mediated translation.

By “internal ribosome entry site” or “IRES” is meant a sequence of nucleotides within a mRNA to which a ribosome or a component thereof, e.g., a 40S subunit of a ribosome is capable of binding. An IRES need not necessarily comprise nucleic acid that induces translation of a mRNA (e.g., a start codon; AUG). In the presence of a suitable start codon, an IRES contemplated by the invention initiates Cap-independent translation. A suitable IRES will be apparent to the skilled artisan and/or is described herein.

As used herein, the term “IRES-mediated translation” shall be taken to mean the translation of a mRNA or open reading frame comprising an IRES that is induced by the binding of a ribosome or a component thereof to the IRES. Accordingly, this term does not encompass Cap-dependent translation as described herein-above.

As used herein, the term “counter-selectable reporter gene” shall be taken to mean a gene encoding a polypeptide that is capable of converting a non-toxic substrate into a substrate that is toxic to a cell in which the counter-selectable reporter gene is expressed. Preferably, the toxic substrate inhibits or reduces the growth of a cell expressing the counter-selectable reporter gene. A suitable counter-selectable reporter gene will be apparent to the skilled artisan and/or described herein. For example, a preferred counter-selectable reporter gene is a gpt gene from Escherichia coli. A cell expressing gpt is incapable of growing in the presence of, for example, 5-thioxanthine. The gpt gene provides an additional advantage of being a positive-selectable marker, e.g., a cell expressing gpt is capable of growing under conditions wherein thioxanthine in the sole source of purine, whereas a cell that does not express gpt is not able to grow under these conditions. Accordingly, in one embodiment, the counter-selectable reporter gene provides for positive and/or negative selection.

As will be apparent to the skilled artisan, only those cells contacted with or comprising a compound capable of reducing or inhibiting IRES-mediated translation are capable of growing in the presence of the substrate of the counter-selectable reporter gene product. Accordingly, the method of the invention enables the rapid identification of a suitable compound as only a cell expressing such a compound is capable of surviving and/or growing under selection conditions.

In the present context, “operably-linked” means that, following transcription, the binding of a ribosome or a component thereof is controlled the IRES. Preferably, when an IRES that is operably linked to a nucleic acid comprising a translation start codon thereby permitting the IRES to control the translation of a protein encoded by the nucleic acid.

Generally, an IRES is positioned 5′ to the coding sequence that it controls, however, the present invention additionally contemplates positioning an IRES both 5′ and 3′ to a sequence that it controls. Some variation in the specific location of the IRES relative to the nucleic acid and/or the translation start site can be accommodated without loss of IRES function. This is because the IRES only provides the site of binding for a ribosome or a component thereof, after which the ribosome or the component scans the linked nucleic acid for a suitable translation start site.

As used herein, the terms “expression”, “expressed” or “express” shall be taken to mean at least the transcription of a nucleotide sequence to produce a RNA molecule. The term “expression” “expressed” or “express” further means the translation of said RNA molecule to produce a peptide, polypeptide or protein.

Preferably, the IRES is from a virus or a mammalian cell, e.g., an IRES that expresses a protein in a cancer cell. Even more preferably, the virus is hepatitis C virus. For example, the IRES comprises the nucleotide sequence set forth in SEQ ID NO: 6.

In one embodiment the method comprises maintaining the cell for a time and under conditions sufficient for the counter selectable reporter gene to be expressed.

In another embodiment, the conditions sufficient to kill a cell expressing the counter-selectable reporter gene are achieved by contacting the cell with a non-toxic substrate that is converted into a cytotoxic substrate by an expression product of the counter-selectable reporter gene.

Preferred counter selectable reporter genes include a gene selected from the group consisting of herpes simplex virus thymidine kinase gene, cytosine deaminase gene, xanthine-guanine phosphoribosyltransferase (gpt) gene, hypoxanthine-guanine phosphoribosyltransferase gene, URA3, CYH2, LYS3 and a D-amino acid oxidase gene.

Preferably, counter selectable reporter gene is a xanthine-guanine phosphoribosyltransferase (gpt) gene.

In a preferred embodiment, the counter selectable reporter gene is a xanthine-guanine phosphoribosyltransferase (gpt) gene and wherein conditions sufficient to kill or inhibit or reduce the growth of a cell expressing the counter-selectable reporter gene are achieved by contacting the cell with thioxanthine or 6-thioguanine.

In one embodiment the method additionally comprises selecting a cell expressing the counter selectable reporter gene prior to contacting the cell with or introducing into the cell a candidate compound under conditions sufficient to kill or inhibit or reduce the growth of a cell expressing the counter-selectable reporter gene.

In accordance with this embodiment, it is preferable that the counter selectable reporter gene is additionally a selectable reporter gene. Suitable reporter genes include, for example, a counter selectable reporter gene selected from the group consisting of a xanthine-guanine phosphoribosyltransferase (gpt) gene, a hypoxanthine-guanine phosphoribosyltransferase gene, URA3, CYH2, LYS3 and a D-amino acid oxidase gene.

In one embodiment, the counter selectable reporter gene is a xanthine-guanine phosphoribosyltransferase (gpt) gene.

Preferably, the method of the invention comprises a first step of maintaining the cell in the presence of xanthine as the sole precursor for guanine nucleotide formation and in the presence of one or more inhibitors that reduce or prevent de novo purine nucleotide synthesis to select a cell expressing the counter-selectable reporter gene.

Preferred compounds for assaying in the method of the invention include, for example, a compound is selected from the group consisting of a peptide, a polypeptide, an antibody, a nucleic acid, a dendrimer and a small molecule. Preferably, the compound is a peptide.

In a preferred embodiment, the peptide is introduced into the cell by means of expressing nucleic acid encoding the peptide. For example, the method of the invention comprises introducing nucleic acid encoding the peptide into the cell.

In one embodiment, the method of the invention described herein according to any embodiment, additionally comprises expressing in the cell a second reporter gene operably linked to a promoter the expression of which is not operably linked to the IRES at (i); and selecting a cell in which the expression of the counter-selectable reporter gene is reduced or inhibited and the expression of the second reporter gene is not detectably reduced or inhibited.

As used herein, the term “promoter” is to be taken in its broadest context and includes the transcriptional regulatory sequences of a genomic gene, including the TATA box or initiator element, which is required for accurate transcription initiation, with or without additional regulatory elements (i.e., upstream activating sequences, transcription factor binding sites, enhancers and silencers) which alter gene expression in response to developmental and/or external stimuli, or in a tissue specific manner. In the present context, the term “promoter” is also used to describe a recombinant, synthetic or fusion molecule, or derivative which confers, activates or enhances the expression of a nucleic acid molecule to which it is operably linked, and which encodes the peptide or protein. Preferred promoters can contain additional copies of one or more specific regulatory elements to further enhance expression and/or alter the spatial expression and/or temporal expression of said nucleic acid molecule.

In the present context, a nucleic acid is placed in “operable connection” with a promoter (i.e., under the regulatory control of a promoter) when it is positioned such that its expression is controlled by the promoter. Promoters are generally positioned 5′ (upstream) to the nucleic acid, the expression of which they control. To construct heterologous promoter/nucleic acid combinations, it is generally preferred to position the promoter at a distance from the gene transcription start site that is approximately the same as the distance between that promoter and the gene it controls in its natural setting, i.e., the gene from which the promoter is derived. As is known in the art, some variation in this distance can be accommodated without loss of promoter function. Similarly, the preferred positioning of a regulatory sequence element with respect to a heterologous nucleic acid to be placed under its control is defined by the positioning of the element in its natural setting, i.e., the gene from which it is derived. Again, as is known in the art, some variation in this distance can also occur.

For example, the second reporter gene is encodes a protein selected from the group consisting of a protein that confers a growth advantage on a cell in which it is expressed, a protein that catalyzes a detectable reaction and a fluorescent protein.

Preferably, the second reporter gene encodes a fluorescent protein, such as, for example, a mutant discosoma red fluorescence protein.

Preferably, the method described herein according to any embodiment additionally comprises obtaining the compound from the cell or providing or producing the compound.

Preferably, the method of the invention additionally comprises providing, producing or obtaining the cell.

In one embodiment, the method of the invention additionally comprises providing, producing or obtaining an expression construct comprising the counter selectable reporter gene in operable connection with the IRES. For example, the method of the invention additionally comprises providing, producing or obtaining an expression construct comprising a second reporter gene operably linked to a promoter.

A preferred construct additionally comprises the IRES operably linked to the counter selectable reporter gene.

In a preferred embodiment, the method of the invention additionally comprises transforming or transfecting the cell with the expression construct.

In one embodiment, a peptide identified using a primary screen of the invention is additionally assayed by performing a method comprising (i.e., the method of the invention additionally comprises):
(i) expressing in a cell a first reporter gene other than a counter-selectable reporter gene operably linked to a promoter and a second reporter gene operably linked to the IRES;
(ii) contacting the cell with or introducing into the cell the identified compound under conditions sufficient for expression of the first and second reporter genes; and
(iii) selecting a cell in which the expression of the first reporter gene is not detectably reduced and the expression of the second reporter gene is reduced or inhibited and said reduced or inhibited expression indicates that the compound that selectively reduces or inhibits IRES-mediated translation.

Preferably, the first and second reporter genes each encode a distinct protein selected from the group consisting of a protein that confers a growth advantage on the cell, a protein that catalyzes a detectable reaction and a fluorescent protein. For example, the first and second reporter genes each encode a fluorescent protein.

In a preferred embodiment, the first reporter gene encodes a green fluorescence protein and the second reporter gene encodes a mutant discosoma red fluorescence protein.

In one embodiment, the method of the invention additionally comprises comparing the level of expression of the first report gene and/or the second reporter gene to the level of expression in the same cell prior to contacting the cell with or introducing into the cell the compound.

Alternatively, the method of the invention additionally comprises comparing the level of expression of the first reporter gene and/or the second reporter gene to a cell that has not been contacted with the compound or had the compound introduced thereto.

Preferably, the method of the invention according to any embodiment comprises:
(i) expressing in a cell a xanthine-guanine phosphoribosyltransferase (gpt) gene counter-selectable reporter gene comprising the nucleotide sequence set forth in SEQ ID NO: 13 operably linked to an IRES;
(ii) expressing in the cell a candidate peptide;
(iii) maintaining the cell in the presence of thioxanthine or 6-thioguanine for a time and under conditions sufficient to kill or inhibit or reduce the growth of a cell expressing the counter-selectable reporter gene; and
(iv) selecting a cell in which the expression of the counter-selectable reporter gene is reduced or inhibited and said reduced or inhibited expression is indicative of reduced or inhibited IRES-mediated translation,
thereby identifying a compound that reduces or inhibits IRES-mediated translation.

In another embodiment, the method as described herein according to any embodiment comprises:
(i) expressing in a cell a xanthine-guanine phosphoribosyltransferase (gpt) gene counter-selectable reporter gene comprising the nucleotide sequence set forth in SEQ ID NO: 13 operably linked to an IRES;
(ii) expressing in the cell a candidate peptide;
(iii) maintaining the cell in the presence of thioxanthine or 6-thioguanine for a time and under conditions sufficient to kill or inhibit or reduce the growth of a cell expressing the counter-selectable reporter gene;
(iv) selecting a cell in which the expression of the counter-selectable reporter gene is reduced or inhibited and said reduced or inhibited expression is indicative of reduced or inhibited IRES-mediated translation and identifying the peptide expressed by the selected cell;
(v) expressing the identified peptide in a cell additionally expressing (a) a fist reporter gene encoding a green fluorescence protein comprising the amino acid sequence set forth in SEQ ID NO: 20 said first reporter gene operably linked to a promoter; and (b) a second reporter gene encoding a mutant discosoma red fluorescence protein comprising an amino acid sequence set forth in SEQ ID NO: 26 said second reporter gene operably linked to the IRES; and
(vi) selecting a cell in which the expression of the first reporter gene is not detectably reduced and the expression of the second reporter gene is reduced or inhibited and said reduced or inhibited expression is indicative of reduced IRES-mediated translation,
thereby identifying a peptide that selectively reduces or inhibits IRES-mediated translation.

The present invention additionally provides a process for providing a compound that inhibits or reduces IRES-mediated translation, said process comprising:
(i) performing the method as described herein according to any embodiment to identify a compound that inhibits or reduces IRES-mediated translation;
(ii) optionally, isolating the compound or a nucleic acid encoding the compound;
(iii) optionally, determining the structure of the compound; and
(iv) providing the compound or the name or structure of the compound.

The present invention additionally provides a cell comprising a counter-selectable reporter gene operably linked to an IRES. For example, the IRES is from a virus or a mammal. Preferably, the IRES is from a hepatitis C virus, e.g., the IRES comprises a nucleotide sequence set forth in SEQ ID NO: 6.

Preferably, the counter selectable reporter gene is selected from the group consisting of herpes simplex virus thymidine kinase gene, a cytosine deaminase gene, a xanthine-guanine phosphoribosyltransferase (gpt) gene, a hypoxanthine-guanine phosphoribosyltransferase gene, URA3, CYH2, LYS3 and a D-amino acid oxidase gene.

More preferably, the counter selectable reporter gene is additionally a selectable reporter gene, e.g., a counter selectable reporter gene is selected from the group consisting of a xanthine-guanine phosphoribosyltransferase (gpt) gene, a hypoxanthine-guanine phosphoribosyltransferase gene, URA3, CYH2, LYS3 and a D-amino acid oxidase gene. Preferably, the counter selectable reporter gene is a xanthine-guanine phosphoribosyltransferase (gpt) gene comprising a nucleotide sequence set forth in SEQ ID NO: 13.

In one embodiment, the cell additionally comprises a second reporter gene operably linked to a promoter. For example, the second reporter gene encodes a protein selected from the group consisting of a protein that confers a growth advantage on the cell, a protein that catalyzes a detectable reaction and a fluorescent protein. Preferably, the second reporter gene encodes a fluorescent protein, e.g., a mutant discosoma red fluorescence protein.

The present invention also provides an expression construct comprising a counter-selectable reporter gene operably linked to an IRES. Preferably, the IRES is from a virus or a mammal, e.g., the IRES is from a hepatitis C virus, e.g., the IRES comprises a nucleotide sequence set forth in SEQ ID NO: 6.

In one embodiment, the counter selectable reporter gene is selected from the group consisting of herpes simplex virus thymidine kinase gene, a cytosine deaminase gene, a xanthine-guanine phosphoribosyltransferase (gpt) gene, a hypoxanthine-guanine phosphoribosyltransferase gene, URA3, CYH2, LYS3 and a D-amino acid oxidase gene.

Preferably, the counter selectable reporter gene is additionally a selectable reporter gene, e.g., a counter selectable reporter gene is selected from the group consisting of a xanthine-guanine phosphoribosyltransferase (gpt) gene, a hypoxanthine-guanine phosphoribosyltransferase gene, URA3, CYH2, LYS3 and a D-amino acid oxidase gene. Preferably, the counter selectable reporter gene is a xanthine-guanine phosphoribosyltransferase (gpt) gene comprising a nucleotide sequence set forth in SEQ ID NO: 13.

In one embodiment, the expression construct additionally comprises a second reporter gene operably linked to a promoter. For example, the second reporter gene encodes a protein selected from the group consisting of a protein that confers a growth advantage on the cell, a protein that catalyzes a detectable reaction and a fluorescent protein. Preferably, the second reporter gene encodes a fluorescent protein, e.g., a mutant discosoma red fluorescence protein.

The present invention additionally provides an isolated or recombinant peptide or peptide analogue selected from the group consisting of:
(i) a peptide consisting of an amino acid sequence selected from the group consisting of SEQ ID NO: 103, SEQ ID NO: 105, SEQ ID NO: 107, SEQ ID NO: 108, SEQ ID NO: 109 and SEQ ID NO: 110, optionally comprising an N-terminal methionine residue;
(ii) a peptide or peptide analogue encoded by a nucleic acid consisting of a sequence selected from the group consisting of SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147 and SEQ ID NO: 148, optionally comprising a 5′ sequence encoding a methionine residue;
(iii) a peptide comprising an amino acid sequence selected from the group consisting of SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 104 and SEQ ID NO: 106;
(iv) a peptide or peptide analogue encoded by a nucleic acid comprising a sequence selected from the group consisting of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73 and SEQ ID NO: 74 SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123 and SEQ ID NO: 124; and
(v) an analogue of any one of (i) to (iv) selected from the group consisting of (a) the sequence of any one of (i) to (iv) comprising one or more non-naturally-occurring amino acids; (b) the sequence any one of (i) to (iv) comprising one or more non-naturally-occurring amino acid analogues; (c) an isostere of any one of (i) to (iv); (d) a retro-peptide analogue of any one of (i) to (iv); and (e) a retro-inverted peptide analogue of any one of (i) to (iv).

Preferably, the isolated or recombinant peptide or peptide analogue is selected from the group consisting of:
(i) a peptide consisting of an amino acid sequence set forth in SEQ ID NO: 103 or SEQ ID NO: 108;
(ii) an analogue of (i) selected from the group consisting of (a) the sequence of (i) comprising one or more non-naturally-occurring amino acids; (b) the sequence of (i) comprising one or more non-naturally-occurring amino acid analogues; (c) an isostere of (i); (d) a retro-peptide analogue of (i); and (e) a retro-inverted peptide analogue of (i).

Preferably, the isolated or recombinant peptide or peptide analogue selected from the group consisting of:
(i) a peptide consisting of an amino acid sequence set forth in SEQ ID NO: 105 or SEQ ID NO: 109;
(ii) an analogue of (i) selected from the group consisting of (a) the sequence of (i) comprising one or more non-naturally-occurring amino acids; (b) the sequence of (i) comprising one or more non-naturally-occurring amino acid analogues; (c) an isostere of (i); (d) a retro-peptide analogue of (i); and (e) a retro-inverted peptide analogue of (i).

Preferably, the isolated or recombinant peptide or peptide analogue is selected from the group consisting of:
(i) a peptide consisting of an amino acid sequence set forth in SEQ ID NO: 107 or SEQ ID NO: 110;
(ii) an analogue of (i) selected from the group consisting of (a) the sequence of (i) comprising one or more non-naturally-occurring amino acids; (b) the sequence of (i) comprising one or more non-naturally-occurring amino acid analogues; (c) an isostere of (i); (d) a retro-peptide analogue of (i); and (e) a retro-inverted peptide analogue of (i).

Preferably, the isolated or recombinant peptide or peptide analogue is selected from the group consisting of:
(i) a peptide comprising an amino acid sequence set forth in SEQ ID NO: 104;
(ii) an analogue of (i) selected from the group consisting of (a) the sequence of (i) comprising one or more non-naturally-occurring amino acids; (b) the sequence of (i) comprising one or more non-naturally-occurring amino acid analogues; (c) an isostere of (i); (d) a retro-peptide analogue of (i); and (e) a retro-inverted peptide analogue of (i).

In one embodiment, the isolated or recombinant peptide or peptide analogue at (iii) or (iv) supra comprises a protein transduction domain. For example, the protein transduction domain is a HIV-1 TAT protein transduction domain.

Preferred analogues comprise one or more D amino acids. For example, each of the amino acids in said analogue is a D amino acid.

Preferably, the analogue of the invention is a retro-inverted peptide analogue. For example, the analogue comprises a reversed sequence of a peptide described herein according to any embodiment and an amino acid residue in said sequence is inverted.

Preferably, the analogue of the invention comprises a reversed sequence of a peptide described herein according to any embodiment and every amino acid residue in said sequence is inverted.

The present invention also provides a pharmaceutical composition comprising the isolated or recombinant peptide or peptide analogue described herein according to any embodiment and a pharmaceutically acceptable carrier or excipient.

The present invention also provides a pharmaceutical composition comprising a nucleic acid that encodes the isolated or recombinant peptide or peptide analogue described herein according to any embodiment and a pharmaceutically acceptable carrier or excipient.

In one embodiment the pharmaceutical composition additionally comprises an antiviral agent.

The present invention also provides a method for providing or producing an isolated or recombinant peptide or peptide analogue described herein according to any embodiment comprising providing or obtaining a sequence of the peptide or peptide analogue or a sequence of nucleic acid encoding the peptide or peptide analogue and synthesizing or expressing the peptide or peptide analogue.

The present invention also provides a method of therapeutic or prophylactic treatment of a subject comprising administering an isolated or recombinant peptide or peptide analogue described herein according to any embodiment to a subject in need thereof. Preferably, the therapeutic or prophylactic treatment comprises treating or preventing a viral infection in a subject.

In one embodiment, the method additionally comprises determining or identifying a subject in need of treatment.

The present invention additionally provides for the use of the isolated or recombinant peptide or peptide analogue described herein according to any embodiment in medicine.

The present invention also provides a method of treating or preventing a viral infection, said method comprising administering one or more peptides or peptide analogues or a pharmaceutical composition comprising said one or more peptides or peptide analogues to a subject in need thereof, wherein a peptide or peptide analogue is selected from the group consisting of:
(i) a peptide comprising an amino acid sequence selected from the group consisting of SEQ ID NO: 103, SEQ ID NO: 105, SEQ ID NO: 107, SEQ ID NO: 108, SEQ ID NO: 109 and SEQ ID NO: 110;
(ii) an analogue of (i) selected from the group consisting of (a) the sequence of (i) comprising one or more non-naturally-occurring amino acids; (b) the sequence of (i) comprising one or more non-naturally-occurring amino acid analogues; (c) an isostere of (i); (d) a retro-peptide analogue of (i); and (e) a retro-inverted peptide analogue of (i).

Preferably, the peptide or peptide analogue comprises an amino acid sequence set forth in SEQ ID NO: 103. Preferably, the peptide or peptide analogue comprises an amino acid sequence set forth in SEQ ID NO: 105. Preferably, the peptide or peptide analogue comprises an amino acid sequence set forth in SEQ ID NO: 107. Preferably, the peptide or peptide analogue comprises an amino acid sequence set forth in SEQ ID NO: 108. Preferably, the peptide or peptide analogue comprises an amino acid sequence set forth in SEQ ID NO: 109. Preferably, the peptide or peptide analogue comprises an amino acid sequence set forth in SEQ ID NO: 110.

The present inventing additionally provides a method of treating or preventing a viral infection, said method comprising administering the peptide or peptide analogue described herein according to any embodiment or the pharmaceutical composition described herein according to any embodiment to a subject in need thereof. Preferably, the subject suffers from a viral infection or is at risk of being infected by a virus.

Preferably, the viral infection is a hepatitis C viral infection.

The present invention also provides for the use of one or more peptides or peptide analogues in the manufacture of a medicament for the treatment of a viral infection, wherein the peptide or peptide analogue is selected from the group consisting of:
(i) a peptide comprising an amino acid sequence selected from the group consisting of SEQ ID NO: 103, SEQ ID NO: 105, SEQ ID NO: 107, SEQ ID NO: 108, SEQ ID NO: 109 and SEQ ID NO: 110;
(ii) an analogue of (i) selected from the group consisting of (a) the sequence of (i) comprising one or more non-naturally-occurring amino acids; (b) the sequence of (i) comprising one or more non-naturally-occurring amino acid analogues; (c) an isostere of (i); (d) a retro-peptide analogue of (i); and (e) a retro-inverted peptide analogue of (i); and
(iii) any other peptide or peptide analogue described herein according to any embodiment.

### BRIEF DESCRIPTION OF THE DRAWINGS

FIG. 1 is a schematic representation of a gene construct used for performance of the screening method of the present invention. The depicted gene construct comprises a nucleic acid encoding the monomer red fluorescent protein dsREDT™ (dsRED) linked to the 5′ site of a internal ribosome entry site (IRES) that is in turn located 5′ to the positive-negative selectable marker xanthine-guanine phosphoribosyltransferase (gpt).

FIG. 2 is a schematic representation of a screening method of the invention, a construct of the invention (e.g., as shown in FIG. 1) is stably integrated into the genome of a cell unable to metabolize xanthine for purine synthesis (e.g. a HEK293 cell). Cells comprising the construct are selected using positive selection (i.e., the ability to metabolize xanthine). A library of test peptides are then transformed or transfected into the selected cells and cells treated with 6-thioxanthine (thereby killing cells expressing gpt). Those cells remaining are considered to express a peptide that inhibits IRES activity and the nucleic acid encoding the peptide isolated using reverse-transcriptase mediated polymerase chain reaction (RT-PCR). The screening of the isolated peptides is optimally repeated a number of times to enrich for those peptides that reproducibly inhibit IRES activity and the identified peptides used in further validation assays.

FIG. 3 is a graphical representation showing the growth of HEK293 cells comprising a construct in which a gpt gene is placed under the control of a HCV IRES or control cells grown in the presence or absence of 6-thioxanthine (100 μM). The number of days of 6-thioxanthine treatment is indicated on the X-axis. The optical density at 590 nm of the tissue culture flask containing the cells is shown on the Y-axis. The OD590 of control cells, i.e., that do not contain the IRES-gpt construct grown in the absence of 6-thioxanthine are represented by the solid circles and dashed line. The OD590 of control cells, i.e., that do not contain the IRES-gpt construct grown in the presence of 6-thioxanthine are represented by the solid circles and solid line. The OD590 of cells comprising the IRES-gpt construct grown in the absence of 6-thioxanthine are represented by solid squares and dashed line. The OD590 of cells comprising the IRES-gpt construct grown in the presence of 6-thioxanthine are represented by solid squares and solid line.

FIG. 4a is a graphical representation showing the level of expression of the eGFP gene operably linked to HCV IRES in a cell expressing the IP 1-04 peptide and in a cell that doesn't express the peptide. The figure shows two peaks of fluorescence detected, with a reduced level in cells expressing the peptide compared to cells not expressing the peptide. This indicates that IP 1-04 reduces IRES-mediated eGFP expression.

FIG. 4b is a graphical representation showing the level of expression of the dsRED2 gene operably linked to a CMV promoter in a cell expressing the IP 1-04 peptide and in a cell that doesn't express the peptide. The figure shows that the two peaks of fluorescence detected are approximately the same indicating that IP 1-04 does not modulate Cap-dependent expression.

### DETAILED DESCRIPTION OF THE PREFERRED EMBODIMENTS

As will be apparent from the description herein, an IRES is generally transcribed in an mRNA transcript and induces translation of a region of the mRNA transcript (i.e., an open reading frame) positioned 3′ to the IRES. Accordingly, in one embodiment, the present invention provides a method for determining a compound that reduces or inhibits IRES-mediated translation, said method comprising:
(i) transcribing in a cell a counter-selectable reporter gene operably linked to an IRES;
(ii) contacting the cell with or introducing into the cell a candidate compound under conditions sufficient to translate the counter-selectable reporter gene transcribed at (i) and under conditions sufficient to kill or inhibit the growth of a cell expressing the counter-selectable reporter gene; and
(iii) selecting a cell in which the translation of the counter-selectable reporter gene is reduced or inhibited and said reduced or inhibited translation is indicative of reduced or inhibited IRES-mediated translation,
   thereby determining a compound that reduces or inhibits IRES-mediated translation.

As will be apparent to the skilled artisan based on the description herein, a cell in which the expression of the counter-selectable reporter gene is reduced or inhibited is “selected” by its ability to grow in the presence of a substrate of a counter-selectable marker that is converted into a toxic substrate and identifying or determining a cell capable of growing.

### 1. Internal Ribosome Entry Sites

At the nucleotide sequence level, IRESs are poorly conserved. An IRES comprises a plurality of ATG start codons and may comprise the ATG start codon that is used as the translational start site by a ribosome (however, this is not a requirement as the HCV IRES does not appear to comprise such a site). Furthermore, an IRES generally comprises a pyrimidine-rich sequence located at or near the 3′ boundary or end of the IRES.

Rather than a specific consensus nucleotide sequence, it is the secondary and/or tertiary structure of a region of nucleic acid that provides the functionality of an IRES (i.e., the site for a ribosome to bind and start scanning for a start codon and commence translation of a mRNA). For example, an IRES generally forms a plurality of stem-loop structures.

A sequence comprising an IRES may be predicted using software that detects regions comprising a high level of RNA secondary structure. For example, the ConStruct software predicts optimal and sub-optimal secondary structures in a single-stranded RNA of a set of homologous RNAs and stores the sequences that form these structures in a base-pair probability matrix. A multiple sequence alignment is performed for the set of RNAs. Any resulting gaps are introduced into the individual probability matrices. These homologous probability matrices are summed to give a consensus probability matrix emphasizing the conserved secondary structure elements of the RNA set. Accordingly, the algorithm used by this software combines the advantages of thermodynamic structure prediction by energy minimization with the information obtained from phylogenetic alignment of sequences. The algorithm used in the ConStruct software is described in Lück et al., J. Mol. Biol. 258:813-26, 1996.

Alternatively, an IRES is predicted on the basis of both RNA secondary structure and thermodynamic stability. For example, the RNAz software is based on the teachings of Washietl et al., Proc. Natl. Acad. Sci. USA 102: 2454-2459 2005 and makes use of two algorithms, the first of which predicts areas of high secondary structure that are conserved in multiple strains or sub-strains of an organism (similar to the method of Lück et al., supra). These regions are then analysed to determine those that have a high degree of thermostability (i.e., z-score) relative to a panel of random sequences. Regions of nucleic acid that are predicted to have a high degree of secondary structure and thermostability are predicted to be functional RNAs, for example, an IRES.

Even following prediction of an IRES using an algorithm, e.g., an algorithm described herein, such a sequence is generally analysed empirically, for example, by deletion studies to determine the minimum sequence required to induce expression of a downstream ORF or that interacts with proteins known to bind IRES sequences, e.g., 40S ribosomal subunit and/or poly(rC) binding protein-1 or -2.

Alternatively, or in addition, the putative IRES is inserted into a bicistronic vector. Preferably, the putative IRES is inserted between two open reading frames in a bicistronic vector. This vector is then transformed or transfected into a cell or analysed using an in vitro translation system and translation of the protein encoded by the open reading frame downstream (i.e., 3′) of the IRES detected. Translation of this protein indicates that the putative IRES is an IRES.

By “bicistronic” is meant a single nucleic acid molecule that is capable of encoding two distinct proteins from different regions of the nucleic acid. In this regard, the two distinct proteins are not encoded by, for example, different splice forms of the same gene, rather each protein is encoded by an entirely distinct region of RNA transcribed from the bicistronic nucleic acid.

Alternatively, Johanns et al, Proc. Natl. Acad. Sci. USA, 96: 13118-13123, 1999 describes a method for identifying a nucleic acid likely to include an IRES. The method involves infecting a cell with a replication defective poliovirus (deficient in IRES activity) to thereby suppress Cap-initiated translation. RNA associated with polysomes is collected from the cells and analysed using a microarray. The sequence of the 5′ untranslated region of an identified RNA molecule is then analysed to determine whether or not it is likely to comprise an IRES using either in silico techniques or by cloning the sequence into a bicistronic vector and analysing for gene expression.

The nucleotide sequences of a number of IRES are known in the art and publicly available. For example, the IRES database (described by Bonnal et al., Nucleic Acids Res. 31: 427-8, 2003) comprises the nucleotide sequence of IRESs from both a viral source and a eukaryotic source. Access to the IRES database is available from The French Institute of Health and Medical Research (INSERM).

By way of exemplification only the nucleotide sequence of an IRES useful for performance of the invention is set forth in any of SEQ ID NOs: 1 to 6. SEQ ID NO: 1 sets forth the nucleotide sequence of an IRES from poliovirus; SEQ ID NO: 2 sets forth the nucleotide sequence of an IRES from human rhinovirus; SEQ ID NO: 3 sets forth the nucleotide sequence of an IRES from hepatitis A virus; SEQ ID NO: 4 sets forth the nucleotide sequence of an IRES from HIV-1; SEQ ID NOs: 5 and 6 set forth the nucleotide sequence from an IRES from HCV; SEQ ID NO: 7 sets forth the nucleotide sequence of an IRES from foot and mouth disease virus; SEQ ID NO: 8 sets forth the nucleotide sequence of an IRES from c-myc; SEQ ID NO: 9 sets forth the nucleotide sequence of an IRES from X-linked inhibitor of apotosis protein (XIAP); SEQ ID NO: 10 sets forth the sequence of an IRES from apoptotic protease activating factor (APAF1); SEQ ID NO: 11 sets forth the sets forth the nucleotide sequence of an IRES from fibroblast growth factor 2 (FGF2); and SEQ ID NO: 12 sets forth the nucleotide sequence of an IRES from vascular endothelial growth factor (VEGF).

### 2. Counter-Selectable Reporter Genes

A suitable counter-selectable reporter gene for use in the method of the present invention will be apparent to those skilled in the art and/or described herein.

For example, should the assay be performed in a mammalian cell, a suitable counter-selectable reporter gene is herpes simplex virus thymidine kinase (TK) gene. In the presence of ganciclovir (an acyclic guanosine analogue) a cell expressing the TK gene converts the analogue to its monophosphate form which is then metabolized into its di- and/or tri-phosphate form by cellular kinases. The triphosphate form of ganciclovir inhibits DNA-α-polymerase and is incorporated into DNA leading to chain termination and cell death.

Alternatively, a counter-selectable marker gene is a cytosine deaminase gene from bacteria or fungi. Cytosine deaminase is useful as a counter-selectable reporter gene in a bacterium, a fungus or a mammalian cell. A cell expressing a cytosine deaminase gene is sensitive to 5-fluorocytosine. In the presence of a cytosine deaminase f-fluorocytosine is deaminated to produce 5-fluorouracil, which is then metabolized by the cell to produce 5-fluorouridine, 5-triphosphate and 5-fluoro-2-deoxyuridine 5-monophosphate each of which reduce or inhibit DNA and/or RNA synthesis thereby inducing cell death.

In a preferred embodiment, the reporter gene used facilitates both positive and negative selection, to thereby facilitate initial selection of a cell that expresses the counter-selectable reporter gene.

As used herein, the term “positive selection” shall be taken to mean placing a cell under conditions sufficient to allow only for a cell expressing a selectable marker (or a counter-selectable marker) to grow. For example, expression a positive selectable marker may confer antibiotic resistance upon a cell, or provide a compound required for survival of a cell under a specific condition.

By “negative selection” is meant placing a cell under conditions sufficient to kill or inhibit or reduce the growth of a cell expressing a counter-selectable marker.

Accordingly, the counter-selectable reporter gene that facilitates both positive and negative selection facilitates the selection of a cell expressing the gene under a set of conditions (i.e., provides a growth advantage to a cell or complements an auxotrophy in a cell) and the reporter gene enables selection against a cell expressing the gene under another set of conditions (i.e., negative selection or counter-selection).

For example, a suitable counter-selectable reporter gene is a modified blasticidin resistance gene (e.g., bsr from Bacillus cereus or BSD from Aspergillus terreus) (described by Karreman et al., Nucl. Acids Res. 26: 2508-2510, 1998). The C-terminus of either of these genes was fused to the HSV-TK gene and the resulting gene encoded a protein that provided resistance to blasticidin and sensitivity to ganciclovir.

Oh et al., Mol. Cells, 11: 192-197, 2001 also describes the production of a nucleic acid encoding a fusion protein that provides for both positive and negative selection. These reporter genes are a fusion of nucleic acids encoding a protein conferring puromycin or hygromycin resistance and green fluorescent protein (GFP) and HSV-TK. The markers provide for positive selection in the presence of either puromycin or hygromycin and also by detection of GFP and for negative selection in the presence of ganciclovir.

Alternatively, the counter-selectable reporter gene is a fusion of the neomycin resistance gene fused to a HSV-TK gene. In this case, the gene confers resistance to various neomycin derivatives in mammalian cells (or Kan in bacteria) and sensitivity to ganciclovir.

In a preferred embodiment, the counter-selectable reporter gene is the E. coli xanthine-guanine phosphoribosyltransferase (gpt) gene (SEQ ID NO: 13). A cell expressing gpt is selected by growth in the presence of xanthine as the sole precursor for guanine nucleotide formation in a medium containing inhibitors (aminopterin and mycophenolic acid) that block de novo purine nucleotide synthesis. Continued expression of the gpt gene is selected against by contacting a cell with thioxanthine or 6-thioguanine. As 6-thioguanine is also toxic to cells expressing endogenous hypoxanthine-guanine phosphoribosyltransferase (HGPRT), thioxanthine is the preferred substrate for negative selection.

In another embodiment, a counter-selectable reporter gene is HPRT or HGPRT, e.g., comprising a nucleotide sequence set forth in SEQ ID NO: 15. A modified cell that does not express functional HPGRT is used for the performance of the invention. A cell expressing HPGRT or HPRT (SEQ ID NO: 16) under control of an IRES is selected using HAT medium (hypoxanthine, guanine and thymidine) and is selected against using medium containing 6-thioguanine. A suitable cell is described, for example, in Fukagawa et al., Nucl. Acids Res., 27: 1966-1969, 1999.

In the case of a screen performed in a yeast cell, a suitable counter-selectable marker is, for example, URA3, CYH2 or LYS3. For example, URA3 expression is selected for in the absence of uracil in culture medium, while the compound 5-fluoro-orotic acid is converted into a toxic compound thereby providing negative selection.

A suitable positive/negative selectable marker gene for use in preforming the method of the invention in a plant cell is, for example, a D-amino acid oxidase gene (dao-1) (SEQ ID NO: 17) encoding the DAAO protein (SEQ ID NO: 18)(Erikson et al., Nat. Biotech., 22: 455-458, 2004. In the absence of dao-1 expression the amino acid D-alanine or D-serine is toxic to a plant or plant cell. However these D-amino acids are metabolised by DAAO to produce non-toxic products, thereby providing for positive selection of a plant cell expressing the marker. In contrast, D-isoleucone or D-valine are metabolised by DAAO into the toxic keto-acids 3-methyl-2-oxopentanoate and 3-methyl-2-oxobutanoate, respectively. However, these D-amino acids are not toxic to a cell that does not express dao-1, thereby providing for negative selection of a plant cell that expresses the marker (i.e., a cell that does not comprise an agent capable of inhibiting RNA-viral IRES-mediated translation).

### Additional Reporter Genes

In another embodiment, the cell used in the method of the present invention additionally expresses a second reporter gene (preferably, a positive selectable maker, e.g., a protein that confers a growth advantage on a cell expressing the gene or a fluorescent protein). The second reporter gene is operably under the control of a promoter. By determining a compound capable of reducing or preventing expression of the counter selectable reporter gene but not the second reporter gene, and IRES inhibitory compound is determined.

Alternatively, or in addition, the second reporter gene facilitates detection of a cell comprising or capable of expressing or expressing a counter selectable reporter gene operably linked to an IRES. For example, such an additional reporter gene is linked to the IRES and counter-selectable marker so as to produce a bicistronic expression construct. Those skilled in the art will be aware that a large number of reporter genes are suitable for practice of the method of the invention. The reporter gene need only be capable of expression and detection in the screening environment, e.g., in the cell in which the method of the invention is performed.

As will be apparent to the skilled artisan from the foregoing, it is preferable that the bicistronic expression construct is capable of expressing a second reporter gene independently of IRES-mediated translation and the counter-selectable reporter gene under control of the IRES. The second reporter gene provides for the ability to select for a cell that comprises the bicistronic expression construct by detection of an expression product of said reporter gene.

Any reporter gene that facilitates selection of a cell comprising an expression construct and expressing the second reporter gene is contemplated as a second reporter gene of the present invention. For example, the reporter gene is an enzyme that catalyzes a detectable reaction. Exemplary enzymatic reporter genes include for example, β-galactosidase, alkaline phosphatase, firefly luciferase or Renilla luciferase. For example, the expression of β-galactosidase is detected by the addition of the substrate 5-bromo-4-chloro-3-indolyl-beta-D-galactopyranoside (x-gal), which is hydrolysed by β-galactosidase to produce a blue coloured precipitate. Alternatively, the expression of either firefly luciferase or Renilla luciferase is detected by addition of a substrate that in the presence of the relevant protein is luminescent and is detectable, for example, using a spectrophotometer.

Alternatively, the reporter gene complements an auxotrophy in a cell. For example, a eukaryotic cell lacking HPRT expression is transformed with an expression construct comprising a nucleic acid encoding HPRT (SEQ ID NO: 16). Expression of the reporter gene results in the cell being capable of growing in HAT medium, while cells that do not express the reporter gene are not capable of growing in these conditions.

Alternatively, in the case of a yeast cell, the reporter gene is, for example, LEU2 or LYS2 or TRP. Such a reporter gene is capable of complementing a yeast cell that is auxotrophic for the relevant gene, and, as a consequence unable to produce the relevant amino acid.

In a preferred embodiment, the reporter gene encodes a polypeptide that is directly detectable (i.e., does not require addition of a substrate or maintaining cells under specific conditions). Preferably, the reporter gene is a fluorescent protein. Several fluorescent reporter genes are known in the art and include, for example, those that encode green fluorescent protein (GFP), enhanced green fluorescent protein (eGFP) (SEQ ID NO: 20), red shifted green fluorescent protein (RFP), cyan fluorescent protein (CFP) (SEQ ID NO: 22), yellow fluorescent protein (YFP) (SEQ ID NO: 24), monomeric discosoma red fluorescent protein (dsRED; SEQ ID NO: 26), or dsRED2 (SEQ ID NO: 28); monomeric orange fluorescent protein (SEQ ID NO: 30) or monomeric GFP from Aequorea coerulescens (SEQ ID NO: 32).

Promoters

To provide for expression of the second reporter gene, it is preferable that the gene is placed in operable connection with a promoter, e.g., that is operable in the cell in which the method of the invention is performed.

In one embodiment, the second reporter gene is placed in operable connection with a promoter that is endogenous to the cell being assayed. For example, the reporter gene is located within the genome of a cell such that it is in operable connection with a promoter endogenous to the cell. For example, the positioning of the reporter gene in a suitable site in the genome of the cell is by random integration of the construct following transfection or transformation of the cell. A cell comprising the construct in a suitable location is then selected by determining whether or not the second reporter construct is expressed.

In another embodiment, the reporter construct is positioned at a previously selected site in the genome of the cell using homologous recombination. Methods of homologous recombination are known in the art and/or described herein. In accordance with these embodiments, the expression construct does not comprise a promoter in operable connection with the second reporter gene.

In one embodiment, the promoter is derived from a gene expressed in the cell to be assayed. Alternatively, the promoter used is any promoter capable of expressing a nucleic acid in the selected cell. For example, a constitutive promoter is used.

A suitable promoter for use in the method of the invention will be apparent to the skilled person and/or described herein. For example, a promoter suitable for expressing a nucleic acid in a mammalian cell includes, for example, a promoter selected from the group consisting of, a retroviral LTR element, a SV40 early promoter, a SV40 late promoter, a cytomegalovirus (CMV) promoter, a CMV E (cytomegalovirus immediate early) promoter, an EF_{1α} promoter (from human elongation factor 1α), an EM7 promoter and a UbC promoter (from human ubiquitin C).

A promoter suitable for expression in insect cells includes, but is not limited to, the OPEI2 promoter, an insect actin promoter isolated from Bombyx muri, a Drosophila sp. dsh promoter (Marsh et al Hum. Mol. Genet. 9, 13-25, 2000) or an inducible metallothionein promoter.

A typical promoter for expressing a nucleic acid in a plant cell is known in the art, and includes, but is not limited to, a Hordeum vulgare amylase gene promoter, a cauliflower mosaic virus 35S promoter, a nopaline synthase (NOS) gene promoter, and an auxin inducible plant promoters P1 and P2.

A suitable promoter for expression in a bacterial cell includes, for example, the lacz promoter, the Ipp promoter, temperature-sensitive λ_{L} or λ_{R} promoters, T7 promoter, T3 promoter, SP6 promoter or semi-artificial promoters such as the IPTG-inducible tac promoter or lacUV5 promoter. A number of other gene construct systems for expressing the nucleic acid fragment of the invention in bacterial cells are known in the art and are described, for example, in Ausubel et al (In: Current Protocols in Molecular Biology. Wiley Interscience, ISBN 047 150338, 1987) and (Sambrook et al (In: Molecular Cloning: Molecular Cloning: A Laboratory Manual, Cold Spring Harbor Laboratories, New York, Third Edition 2001).

A typical promoter for expressing a peptide, polypeptide or protein, e.g., as encoded by a reporter gene in a yeast cell, includes, for example, an ADH1 promoter, a GAL1 promoter, a GAL4 promoter, a CUP1 promoter, a PHO5 promoter, a nmt promoter, a RPR1 promoter, or a TEF1 promoter.

### 3. Expression Constructs

Preferably, an IRES operably linked to a counter selectable reporter gene is provided in the form of an expression construct.

As used herein, the term “expression construct” refers to a nucleic acid molecule that has the ability to confer expression on a nucleic acid (e.g. a reporter gene and/or a counter-selectable reporter gene) to which it is operably connected, in a cell. Within the context of the present invention, it is to be understood that an expression construct may comprise or be a plasmid, bacteriophage, phagemid, cosmid, virus sub-genomic or genomic fragment, or other nucleic acid capable of maintaining and/or replicating heterologous DNA in an expressible format.

In one embodiment, the genetic construct of the invention comprises an IRES placed in operable connection with a counter-selectable reporter gene. Such a genetic construct may then be introduced into a cell in operable connection with an endogenous promoter to thereby enable expression of the reporter gene. For example, the genetic construct is introduced 3′- or downstream of an endogenous gene in the cell to effectively produce a bicistronic gene. Preferably, the genetic construct is located between the endogenous gene and the polyadenylation signal of that gene to ensure translation of the reporter gene is induced by the IRES. Such location of the genetic construct may be by random integration or by homologous recombination. Suitable methods for location of the genetic construct will be apparent to the skilled artisan and/or described herein.

A suitable IRES and/or counter selectable reporter gene will be apparent to the skilled person and/or is described supra.

The expression construct may equally comprise a promoter in operable connection with the second reporter gene to thereby induce expression of said reporter gene. Accordingly, in one embodiment, the expression construct additionally comprises a promoter operable in a cell in which an assay of the invention is performed placed in operable connection with the second reporter gene.

A suitable genetic construct for use in the performance of the invention will be apparent to the skilled artisan and/or described herein.

Constructing an Expression Constructs

Methods for the construction of a suitable expression construct for performance of the invention will be apparent to the skilled artisan and are described, for example, in Ausubel et al (In: Current Protocols in Molecular Biology. Wiley Interscience, ISBN 047 150338, 1987) or Sambrook et al (In: Molecular Cloning: Molecular Cloning: A Laboratory Manual, Cold Spring Harbor Laboratories, New York, Third Edition 2001).

For example, each of the components of the expression construct is amplified from a suitable template nucleic acid using, for example, PCR and subsequently cloned into a suitable expression construct, such as for example, a plasmid or a phagemid. Alternatively, the nucleic acid required for the assay is, for example, excised from a suitable source, for example, using a restriction endonuclease and cloned into a suitable expression construct.

One form of expression construct suitable for performance of the invention remains episomal, i.e., does not integrate into the genome of a host cell. Such an expression construct is, for example, a plasmid or a phagemid. Preferably, the expression construct comprises a selectable marker (which may be additional to the selectable markers used in the method of the invention) to enable selection of a cell comprising the construct. A suitable selectable marker will be apparent to the skilled artisan and/or described herein (for example, an antibiotic resistance gene).

Vectors suitable for such an expression construct are known in the art and/or described herein. For example, an expression vector suitable for the method of the present invention in a mammalian cell is, for example, a vector of the pcDNA vector suite supplied by Invitrogen, a vector of the pCI vector suite (Promega), a vector of the pCMV vector suite (Clontech), a pM vector (Clontech), a pSI vector (Promega), a VP16 vector (Clontech) or a vector of the pcDNA vector suite (Invitrogen).

An expression vector suitable for performing the present invention is a plant is, for example, pSS, pB1121 (Clontech), pZ01502, and pPCV701 (Kuncz et al, Proc. Natl. Acad. Sci. USA, 84 131-135, 1987).

Vectors suitable for expressing a nucleic acid in an insect cell are known in the art and commercially available and include, for example, a vector of the pIEX vector suite from Merck or the pAc5 vector suite from Invitrogen.

Vectors suitable for expressing a nucleic acid in a bacterial cell include, for example, PKC30 (Shimatake and Rosenberg, Nature 292, 128, 1981); pKK173-3 (Amann and Brosius, Gene 40, 183, 1985), pET-3 (Studier and Moffat, J. Mol. Biol. 189, 113, 1986); the pCR vector suite (Invitrogen), pGEM-T Easy vectors (Promega), the pL expression vector suite (Invitrogen).

Vectors suitable for expression in a yeast cell include, for example, the pACT vector (Clontech), the pDBleu-X vector, the pPIC vector suite (Invitrogen), the pGAPZ vector suite (Invitrogen), the pHYB vector (Invitrogen), the pYD1 vector (Invitrogen), and the pNMT1, pNMT41, pNMT81 TOPO vectors (Invitrogen), the pPC86-Y vector (Invitrogen), the pRH series of vectors (Invitrogen), pYESTrp series of vectors (Invitrogen).

The skilled artisan will be aware of additionally vectors and sources of such vectors, such as, for example, Invitrogen Corporation, Clontech or Promega.

Alternatively, the expression construct of the invention is produced such that it integrates into the genome of a host cell. Such an expression construct provides several advantages, such as, for example, the construct replicates with the cells native nucleic acid and, as a consequence further transfection or transformation of the expression construct is not required. Furthermore, a stable cell line that expresses a consistent level of a reporter gene operably linked to an IRES may be produced. Such a cell line reduces inter-assay variation when performing the method of the invention.

In one embodiment, a previously described expression construct is used to produce a stably transformed or transfected cell line. For example, the construct is linearized (if necessary) and transfected or transformed into a suitable cell. A cell comprising the expression construct and/or expressing the counter-selectable marker operably linked to an IRES is selected and maintained under selection for a plurality of population doublings. Stable integration of the expression construct into the host cell genome may then be determined using, for example, Southern blotting.

Alternatively, in the case of an expression construct that comprises a second reporter gene that is not in operable connection with a promoter, selection of a cell expressing the reporter gene indicates that the expression construct has integrated into a host cell genome. This is because the expression of the second reporter gene is likely to be caused by the integration of the expression construct such that the gene is placed in operable connection with a promoter endogenous to the cell.

Alternatively, an expression construct of the invention is a viral vector. Suitable viral vectors are known in the art and commercially available. Conventional viral based systems for the delivery of a nucleic acid and integration of that nucleic acid into a host cell genome include, for example, a retroviral vector, a lentiviral vector or an adeno-associated viral vector. Alternatively, an adenoviral vector is useful for introducing a nucleic acid that remains episomal into a host cell. Viral vectors are an efficient and versatile method of gene transfer in target cells and tissues. Additionally, high transduction efficiencies have been observed in many different cell types and target tissues.

The tropism of a retrovirus can be altered by incorporating foreign envelope proteins, expanding the potential target population of target cells. A lentiviral vector is a retroviral vector that is capable of transducing or infecting a non-dividing cell and typically produces high viral titers. Selection of a retroviral gene transfer system depends on the target tissue.

A retroviral vector generally comprises cis-acting long terminal repeats (LTRs) with packaging capacity for up to 6-10 kb of foreign sequence. The minimum cis-acting LTRs are sufficient for replication and packaging of a vector, which is then used to integrate the expression construct into the target cell to provide long term expression. Widely used retroviral vectors include those based upon murine leukemia virus (MuLV), gibbon ape leukemia virus (GaLV), simian immunodeficiency virus (SIV), human immunodeficiency virus (HrV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:274-2378 (1989); Miller et al., J. Virol. 65:2220-2224 (1991); PCT/US94/05700; Miller and Rosman BioTechniques 7:980-990, 1989; Miller, A. D. Human Gene Therapy 1:5-14, 1990; Scarpa et al) Virology 180:849-852, 1991; Burns et al. Proc. Natl. Acad. Sci. USA 90:8033-8037, 1993.).

Various adeno-associated virus (AAV) vector systems have also been developed for nucleic acid delivery. AAV vectors can be readily constructed using techniques known in the art. See, e.g., U.S. Pat. Nos. 5,173,414 and 5,139,941; International Publication Nos. WO 92/01070 and WO 93/03769; Lebkowski et al. Molec. Cell. Biol. 8:3988-3996, 1988; Vincent et al. (1990) Vaccines 90 (Cold Spring Harbor Laboratory Press); Carter Current Opinion in Biotechnology 3:533-539, 1992; Muzyczka. Current Topics in Microbiol. and Immunol. 158:97-129, 1992; Kotin, Human Gene Therapy 5:793-801, 1994; Shelling and Smith Gene Therapy 1:165-169, 1994; and Zhou et al. J. Exp. Med. 179:1867-1875, 1994.

Additional viral vectors useful for delivering an expression construct of the invention include, for example, those derived from the pox family of viruses, such as vaccinia virus and avian poxvirus or an alphavirus or a conjugate virus vector (e.g. that described in Fisher-Hoch et al., Proc. Natl. Acad. Sci. USA 86:317-321, 1989).

Homologous recombination is also a useful method for targeted integration of an expression construct of the invention. For example, an expression construct of the invention comprising a counter-selectable marker operable linked to an IRES is integrated into the genome of a cell with an endogenous gene to thereby produce a bicistronic gene. In this regard, it is preferred that the expression construct is placed following the translation termination signal in the endogenous gene, but before the polyadenylation signal of the endogenous gene.

In another embodiment, an expression construct of the invention comprising a second reporter gene and an IRES operably linked to a counter selectable marker is integrated into the genome of a cell in operable connection with an endogenous promoter. Such integration may be achieved using, for example, homologous recombination to replace an endogenous gene or part thereof. Alternatively, the expression construct is inserted between an endogenous promoter and a gene to which it is operable connected in nature.

Methods for producing a vector for homologous recombination are known in the art and described, for example, in Hogan et al (In: Manipulating the Mouse Embryo. A Laboratory Manual, 2^{nd} Edition. Cold Spring Harbour Laboratory. ISBN: 0879693843, 1994. Homologous recombination is a reaction between any pair of DNA sequences having a similar sequence of nucleotides, where the two sequences interact (recombine) to form a new recombinant DNA species. The frequency of homologous recombination increases as the length of the shared nucleotide DNA sequences increases, and is higher with linearized plasmid molecules than with circularized plasmid molecules. Homologous recombination can occur between two DNA sequences that are less than identical, but the recombination frequency declines as the divergence between the two sequences increases.

A homologous recombination vector generally comprises a nucleic acid of interest flanked by nucleic acids that comprise a nucleotide sequence highly homologous to nucleic acid at the site at which the expression construct is to be integrated. Each of the regions of homology in the nucleic acid construct (i.e., the arms) are preferably of sufficient length to enable integration of the construct into a specific site in the genome of a cell. Such a vector may be an insertion vector (i.e., wherein the two arms comprise nucleotide sequences are adjacent in nature) or alternatively a replacement vector (i.e., wherein the two arms comprise nucleotide sequences are homologous to regions of nucleic acid that are not adjacent in nature and following homologous recombination the nucleic acid between these regions is replaced by the expression construct).

Preferably, the expression construct used in a homologous recombination reaction comprises a selectable marker. In this regard, the selectable marker may be additional to that used to test for IRES activity (e.g., a counter selectable marker). Following introduction of a suitable construct into a cell, e.g., using a method described herein, a cell comprising the expression construct is selected. Preferably, a cell that comprises the expression construct integrated into the site of the cell's genome of interest, for example, using Southern hybridization or using positive-negative selection as described, for example, in U.S. Pat. No. 5,464,764 or U.S. Pat. No. 6,284,541. Suitable methods for performing homologous recombination in a cellular system are known in the art and reviewed, for example, in Bunz, Current Opinion in Oncology, 14: 73-78, 2002.

Introducing an Expression Construct into a Cell

Methods for introducing an expression construct of the invention will be apparent to the skilled artisan. The technique used for a given cell depends on the known successful techniques. Means for introducing recombinant DNA into animal cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

### 4. Suitable Cells

A cell used in the performance of the present invention is any cell in which an IRES to be tested is active, i.e., a cell in which the IRES is capable of binding a ribosome or component thereof and inducing translation of a nucleic acid (comprising a suitable translation initiation codon) operably linked thereto. Accordingly, it is preferred that the cell comprise proteins required for translation of a protein from a mRNA comprising an IRES and preferably, proteins required for Cap-dependent translation. On this basis, it will be apparent to the skilled artisan that the method of the invention may be performed using almost any cell to screen for a candidate IRES inhibitory compound. Preferably, the cell is a eukaryotic cell.

Methods for determining a cell in which an IRES is active will be apparent to the skilled artisan. For example, a cell is transformed or transfected with a nucleic acid construct comprising a bicistronic gene (or multicistronic gene) with an IRES located between two open reading frames (ORFs). Usually, a bicistronic gene useful for determining a cell in which an IRES is active comprises two regions of nucleic acid arranged end to end with an IRES located between the two regions. The vector may comprise a promoter located 5′ to the bicistronic gene or may rely on integration of the nucleic acid in operable connection with an endogenous promoter of the cell.

By transforming or transfecting a cell with the bicistronic gene construct and detecting expression of both gene products encoded by the gene a cell in which an IRES is active is determined. Preferably, the bicistronic gene construct comprises two or more detectable marker genes to facilitate detection of the gene products.

In the case of an IRES active in a mammalian cell a preferred cell in which to perform the method of the invention is, for example, an epithelial cell, a fibroblast, a kidney cell or a T cell. For example, the method of the invention is performed using a cell line selected from the group consisting of COS, CHO, murine 10T, MEF, NIH3T3, MDA-MB-231, MDCK, HeLa, K562, HEK 293 and 293T. Preferably, the cell is a HEK 293 cell.

A compound for inhibiting an IRES that is active in a plant cell is assayed using, for example, a plant cell line selected from the group consisting of a Hordeum vulgare PC-1118 cell line, a Triticum, aestivum PC0998 cell line or a Zea mays PC cv. Zenit PC-1117 cell line.

As several IRESs are also active in an insect cell, the method of the invention may equally be performed with an IRES active in such a cell using, for example, a BT1-TN-5B1-4 cell or a Spodoptera frugiperda cell (eg., a sf19 cell or a sf21 cell).

A cell that is derived from an organism in which the IRES being studied is operable is also useful for performance of the invention. For example, should the IRES being studied be derived from a virus, a suitable cell (or cell-type) for performance of the method of the invention is a cell that the virus infects in nature. For example, an assay to identify an inhibitor of HCV IRES-mediated transcription is performed in a cell or cell line derived from a liver (e.g., a HepG2 cell or a hepatoma cell).

As will be apparent to the skilled artisan, a cell that is suitable for performance of the invention may require modification to facilitate expression of a counter-selectable reporter gene under control of a RNA-virus IRES. Accordingly, it is preferable that the method of the invention additionally comprises providing or producing a cell expressing a counter-selectable reporter gene operably linked to an internal ribosome entry site (IRES).

### 5. Suitable Compounds

The range of compounds contemplated herein for reducing or inhibiting IRES-mediated translation include peptides, peptidomimetics, nucleic acid aptamers, peptide aptamers, dendrimers, antibodies, small organic molecules, such as, for example derived from publicly available combinatorial libraries or nucleic acids.

Preferably, a compound identified in a screen of the invention is capable of binding to an IRES and inhibiting translation mediated therefrom. The compound may bind to an IRES via any means including, for example, a hydrophobic interaction, a hydrogen bond, an electrostatic interaction, a van der Waals interaction, pi stacking, a covalent bond, or a magnetic interaction amongst others.

Peptides

In a preferred embodiment of the invention, a compound screened or assayed using the method of the invention is a peptide, for example, the method is used to screen a peptide library. A peptide is particularly preferred as it provides a larger interaction interface with an IRES than, for example, small molecule.

As used herein, the term “interaction interface” shall be taken to mean the surface of a compound that binds to, forms a bond with or interacts with an IRES. Accordingly, a compound with a large interaction interface forms a plurality of bonds with an IRES, thereby producing a stable interaction that is less susceptible to destabilisation by changing a single nucleotide or the structure of a small region of the IRES as may occur, for example, in an IRES in a retrovirus (e.g., a virus with a high level of mutation).

Such a peptide is produced using any means known in the art. For example, a peptide is produced synthetically. Synthetic peptides are prepared using known techniques of solid phase, liquid phase, or peptide condensation, or any combination thereof, and can include natural and/or unnatural amino acids. Amino acids used for peptide synthesis may be standard Boc (Nα-amino protected Nα-t-butyloxycarbonyl) amino acid resin with the deprotecting, neutralization, coupling and wash protocols of the original solid phase procedure of Merrifield, J. Am. Chem. Soc., 85:2149-2154, 1963, or the base-labile Nα-amino protected 9-fluorenylmethoxycarbonyl (Fmoc) amino acids described by Carpino and Han, J. Org. Chem., 37:3403-3409, 1972. Both Fmoc and Boc Nα-amino protected amino acids can be obtained from various commercial sources, such as, for example, Fluka, Bachem, Advanced Chemtech, Sigma, Cambridge Research Biochemical, Bachem, or Peninsula Labs.

Alternatively, a synthetic peptide is produced using a technique known in the art and described, for example, in Stewart and Young (In: Solid Phase Synthesis, Second Edition, Pierce Chemical Co., Rockford, Ill. (1984) and/or Fields and Noble (Int. J. Pept. Protein Res., 35:161-214, 1990), or using an automated synthesizer. Accordingly, peptides of the invention may comprise D-amino acids, a combination of D- and L-amino acids, and various unnatural amino acids (e.g., β-methyl amino acids, Cα-methyl amino acids, and Nα-methyl amino acids, etc) to convey special properties. Synthetic amino acids include ornithine for lysine, fluorophenylalanine for phenylalanine, and norleucine for leucine or isoleucine.

In another embodiment, a peptide is produced using recombinant means. For example, an oligonucleotide or other nucleic acid is placed in operable connection with a promoter. Methods for producing such expression constructs, introducing an expression construct into a cell and expressing and/or purifying the expressed peptide, polypeptide or protein are known in the art and described supra.

Alternatively, the peptide, polypeptide or protein is expressed using a cell free system, such as, for example, the TNT system available from Promega. Such an in vitro translation system is useful for screening a peptide library by, for example, ribosome display, covalent display or mRNA display.

In a preferred embodiment, a peptide library is screened to identify a compound that inhibits or reduces IRES-mediated translation. By “peptide library” is meant a plurality of peptides that may be related in sequence and/or structure or unrelated (e.g., random) in their structure and/or sequence. Suitable methods for production of such a library will be apparent to the skilled artisan and/or described herein.

For example, a random peptide library is produced by synthesizing random oligonucleotides of sufficient length to encode a peptide of desired length, e.g., 7 or 9 or 15 amino acids. Methods for the production of an oligonucleotide are known in the art. For example, an oligonucleotide is produced using standard solid-phase phosphoramidite chemistry. Essentially, this method uses protected nucleoside phosphoramidites to produce a short oligonucleotide (i.e., up to about 80 nucleotides). Typically, an initial 5′-protected nucleoside is attached to a polymer resin by its 3′-hydroxy group. The 5′hydroxyl group is then de-protected and the subsequent nucleoside-3′-phosphoramidite in the sequence is then coupled to the de-protected group. The internucleotide bond is then formed by oxidising the linked nucleosides to form a phosphotriester. By repeating the steps of de-protection, coupling and oxidation an oligonucleotide of desired length and sequence is obtained. Suitable methods of oligonucleotide synthesis are described, for example, in Caruthers, M. H., et al., “Methods in Enzymology,” Vol. 154, pp. 287-314 (1988).

Each of the oligonucleotides is then inserted into an expression construct (in operable connection with a promoter) and introduced into a cell of the invention. Suitable methods for producing a random peptide library are described, for example, in Oldenburg et al., Proc. Natl. Acad. Sci. USA 89:5393-5397, 1992; Valadon et al., J. Mol. Biol., 261:11-22, 1996; Westerink Proc. Natl. Acad. Sci USA., 92:4021-4025, 1995; or Felici, J. Mol. Biol., 222:301-310, 1991.

Optionally, the nucleic acid is positioned so as to produce a fusion protein, wherein the random peptide is conformationally constrained within a scaffold structure, eg., a thioredoxin (Trx) loop (Blum et al. Proc. Natl. Acad. Sci. USA, 97, 2241-2246, 2000) or a catalytically inactive staphylococcal nuclease (Norman et al, Science, 285, 591-595, 1999), to enhance their stability. Such conformational constraint within a structure has been shown, in some cases, to enhance the affinity of an interaction between a random peptide and its target (e.g., an IRES), presumably by limiting the degrees of conformational freedom of the peptide, and thereby minimizing the entropic cost of binding. A suitable conformationally constrained peptide library is also exemplified herein.

In a preferred embodiment, a peptide library for use in the method of the invention is produced essentially as described in USSN 20030215846 or PCT/AU2004/000214. The method described in these applications is predicated on the understanding that a domain of a protein is capable of forming a stable secondary structure that is capable of binding, with high affinity, to a target nucleic acid, e.g., an IRES.

The libraries described in US 20030215846 or PCT/AU2004/000214 (incorporated herein by reference) are constructed from nucleic acid fragments comprising genomic DNA, cDNA, or amplified nucleic acid derived from one or two or more well-characterized genomes. By “well-characterized” is meant that the genome is substantially sequenced, for example, at least about 60 percent sequenced, or 80 percent sequenced or 90 percent sequenced.

The well-characterized genomes used in the production of an expression library are preferably, a compact genome of a eukaryote (ie. protist, dinoflagellate, alga, plant, fungus, mould, invertebrate, vertebrate, etc). As used herein, the term “compact genome” shall be taken to mean any organism, preferably of the superkingdom Eukaryota that has a haploid genome size of less than about 1700 mega base pairs (Mbp), and preferably, less than 100 Mbp. Exemplary eukaryotes comprising a compact genome include, for example, a eukaryote selected from the group consisting of Arabidopsis thaliana, Anopheles gambiae, Caenorhabditis elegans, Danio rerio, Drosophila melanogaster, Takifugu rubripes, Cryptosporidium parvum, Trypanosoma cruzii, Saccharomyces cerevesiae, and Schizosaccharomyces pombe.

Alternatively, or in addition one or more well-characterized genomes is a compact genome of a prokaryote (ie. bacteria, eubacteria, cyanobacteria, etc) such as, for example a prokaryote selected from the group consisting of Archaeoglobus fulgidis, Aquifex aeolicus, Aeropyrum pernix, Bacillus subtilis, Bordetella pertussis TOX6, Borrelia burgdorferi, Chlamydia trachomatis, Escherichia coli K12, Haemophilus influenzae (rd), Helicobacter pylori, Methanobacterium thermoautotrophicum, Methanococcus jannaschii, Mycoplasma pneumoniae, Neisseria meningitidis, Pseudomonas aeruginosa, Pyrococcus horikoshii, Synechocystis PCC 6803, Thermoplasma volcanium and Thermotoga maritima.

To increase the diversity of the peptides encoded by the expression library, nucleic acid fragments are selected that are from mixtures of organisms, preferably those organisms that are not normally found together in nature. When using fragments from a variety of genomes, it is preferred that the fragments are mixed so as to ensure equal representation from each genome. Accordingly, fragments from each genome are mixed, for example, in accordance with the size of each genome (e.g., in equimolar amounts).

It is to be understood that the nucleic acid fragments used in the production of the expression libraries of the present invention are generated using art-recognized methods such as, for example, a method selected from the group consisting mechanical shearing, digestion with a nuclease and digestion with a restriction endonuclease. Combinations of such methods can also be used to generate the genome fragments. In a particularly preferred embodiment, copies of nucleic acid fragments from one or two or more genomes are generated using polymerase chain reaction (PCR) using random oligonucleotide primers. Preferably, a nucleic acid fragment is of sufficient size to encode a protein domain and/or subdomain.

The nucleic acid fragments or cDNA or amplified DNA derived therefrom are inserted into a suitable vector or expression construct in operable connection with a suitable promoter for expression of each peptide in the diverse nucleic acid sample. This construct is then, for example, introduced into a cell of the invention and screened to identify or determine a peptide capable of inhibiting or reducing IRES-mediated translation.

In another embodiment, a peptide library is produced and a cell of the invention contacted with a peptide from the library. A peptide that is capable of modulating IRES-mediated translation without entering the cell or, alternatively, is capable of entering the cell and reducing or inhibiting IRES-mediated translation is then selected using the method of the invention. Suitable methods for producing a peptide for contacting to a cell of the invention will be apparent to the skilled artisan and include, for example, synthetic means or in vitro transcription and/or translation.

To facilitate peptide entry into the cell, the peptide may be conjugated to (e.g., expressed as a fusion with) a protein transduction domain. As used herein, the term “protein transduction domain” shall be taken to mean a peptide or protein that is capable of enhancing, increasing or assisting penetration or uptake of a compound conjugated to the protein transduction domain into a cell either in vitro or in vivo. Those skilled in the art will be aware that synthetic or recombinant peptides can be delivered into cells through association with a protein transduction domain such as the TAT sequence from HIV or the Penetratin sequence from the Antenapaedia homeodomain protein (see, for example, Temsamani and Vidal, Drug Discovery Today 9: 1012-1019, 2004, for review).

A suitable protein transduction domain will be apparent to the skilled artisan and includes, for example, HIV-1 TAT fragment 48-60 (GRKKRRQRRRG, SEQ ID NO: 33), signal sequence based peptide 1 (GALFLGWLGAAGSTMGAWSQPKKKRKV, SEQ ID NO: 34), signal sequence based peptide 2 (AAVALLPAVLLALLAP, SEQ ID NO: 35), transportan (GWTLNSAGYLLKINLKALAALAKKIL, SEQ ID NO: 36), amphiphilic model peptide (KLALKLALKALKAALKLA, SEQ ID NO: 37), polyarginine (e.g., RRRRRRRRRRR, SEQ ID NO: 38).

Antibodies

The present invention additionally contemplates an antibody-based compound, including an antibody or fragment thereof. Preferably, a library of antibody-based compounds that are capable of being expressed in or entering a cell is screened using the method of the invention.

By “antibody-based compound” is meant an antibody or a fragment thereof, whether produce using standard or recombinant techniques. Accordingly, an antibody-based compound includes, for example, an intact monoclonal or polyclonal antibody, an immunoglobulin (IgA, IgD, IgG, IgM, IgE) fraction, a humanized antibody, or a recombinant single chain antibody, as well as a fragment of an antibody, such as, for example Fab, F(ab)2, and Fv fragments.

For example, a monoclonal antibody against an IRES is produced by immunizing an animal, e.g., a mouse with nucleic acid comprising an IRES or a fragment thereof. Optionally, the nucleic acid is injected in the presence of an adjuvant, such as, for example Freund's complete or incomplete adjuvant, lysolecithin and/or dinitrophenol to enhance the immune response to the immunogen. Spleen cells are then obtained from the immunized animal. The spleen cells are then immortalized by, for example, fusion with a myeloma cell fusion partner, preferably one that is syngenic with the immunized animal. A variety of fusion techniques may be employed, for example, the spleen cells and myeloma cells may be combined with a nonionic detergent or electrofused and then grown in a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and growth media in which the cells have been grown is tested for the presence of binding activity against the polypeptide (immunogen). Hybridomas having high reactivity and specificity are preferred.

Monoclonal antibodies are isolated from the supernatants of growing hybridoma colonies using methods such as, for example, affinity purification using an IRES to isolate an antibody capable of binding thereto. In addition, various techniques may be employed to enhance the yield, such as injection of the hybridoma cell line into the peritoneal cavity of a suitable vertebrate host, such as a mouse. Monoclonal antibodies are then harvested from the ascites fluid or the blood of such an animal subject. Contaminants are removed from the antibodies by conventional techniques, such as chromatography, gel filtration, precipitation, and/or extraction.

A suitable method for producing an anti-nucleic acid antibody will be apparent to the skilled artisan and/or described, for example, in Wang et al., Immunol Lett., 73: 29-34, 2000.

To ensure that the antibody is capable of entering a cell and bind to an IRES thereby inhibiting or reducing IRES-mediated translation an antibody may be conjugated to a protein transduction domain, for example, a protein transduction domain described herein.

Alternatively, an antibody that is expressed within a cell is used for the screening method of the invention. Such an antibody, also known as an intrabody, is essentially a recombinant ScFv antibody fragment. Essentially, an ScFv antibody fragment is a recombinant single chain molecule containing the variable region of a light chain of an antibody and the variable region of a heavy chain of an antibody, linked by a suitable, flexible polypeptide linker.

A library of ScFv fragments is produced, for example, by amplifying the variable regions of a large and/or small chain from nucleic acid encoding an immunoglobulin (for example, using nucleic acid from a spleen cell that may or may not be derived from a subject (e.g., a mouse) that has been previously immunized with an IRES). These regions are cloned into a vector encoding a suitable framework including a linker region to facilitate expression of an intrabody that is stable when expressed in a cell (for example, see Worn et al., J. Biol. Chem., 275: 2795-803, 2003). An intrabody may be directed to a particular cellular location or organelle, for example by constructing a vector that comprises a polynucleotide sequence encoding the variable regions of an intrabody that may be operatively fused to a polynucleotide sequence that encodes a particular target antigen within the cell (see, e.g., Graus-Porta et al, Mol. Cell. Biol. 15:1182-91, 1995; Lener et al., Eur. J. Biochem. 267:1196-205 2000).

Small Molecules

Techniques for synthesizing small organic compounds will vary considerably depending upon the compound, however such methods will be well known to those skilled in the art. In one embodiment, informatics is used to select suitable chemical building blocks from known compounds, for producing a combinatorial library. For example, QSAR (Quantitative Structure Activity Relationship) modelling approach uses linear regressions or regression trees of compound structures to determine suitability. The software of the Chemical Computing Group, Inc. (Montreal, Canada) uses high-throughput screening experimental data on active as well as inactive compounds, to create a probabilistic QSAR model, which is subsequently used to select lead compounds. The Binary QSAR method is based upon three characteristic properties of compounds that form a “descriptor” of the likelihood that a particular compound will or will not perform a required function: partial charge, molar refractivity (bonding interactions), and logP (lipophilicity of molecule). Each atom has a surface area in the molecule and it has these three properties associated with it. All atoms of a compound having a partial charge in a certain range are determined and the surface areas (Van der Walls Surface Area descriptor) are summed. The binary QSAR models are then used to make activity models or ADMET models, which are used to build a combinatorial library. Accordingly, lead compounds identified in initial screens, can be used to expand the list of compounds being screened to thereby identify highly active compounds.

Nucleic Acids

In another embodiment, the method of the invention is used to screen a nucleic acid compound, such as, for example, a siRNA molecule, an antisense molecule, a ribosome or a nucleic acid aptamer.

An anti-sense compound shall be taken to mean an oligonucleotide comprising DNA or RNA or a derivative thereof (e.g., PNA or LNA) that is complementary to at least a portion of a specific nucleic acid (e.g., an IRES). The anti-sense molecule inhibits binding of a ribosome to an IRES or translation induced by an IRES. Preferably, an antisense molecule comprises at least about 15 or 20 or 30 or 40 nucleotides complementary to the sequence of an IRES, for example, an IRES set forth in any one of SEQ ID NOs: 1 to 11. The use of antisense methods is known in the art (Marcus-Sakura, Anal. Biochem. 172: 289, 1988).

A nucleic acid aptamer (adaptable oligomer) is a nucleic acid molecule that is capable of forming a secondary and/or tertiary structure that provides the ability to bind to a molecular target. For example, an aptamer is produced that is capable of binding to an IRES and inhibiting IRES-mediated translation. An aptamer library is produced, for example, by cloning random oligonucleotides into a vector (or an expression vector in the case of an RNA aptamer), wherein the random sequence is flanked by known sequences that provide the site of binding for PCR primers. An aptamer that provides the desired biological activity (e.g., suppresses IRES-mediated expression) is selected. An aptamer with increased activity is selected, for example, using SELEX (Sytematic Evolution of Ligands by EXponential enrichment). Suitable methods for producing and/or screening an aptamer library are described, for example, in Elloington and Szostak, Nature 346:818-22, 1990.

A ribozyme is an antisense nucleic acid molecule that is capable of specifically binding to and cleaving a target nucleic acid (e.g., an IRES). A ribozyme that binds to an IRES and cleaves this sequence reduced or inhibits the ability of a ribosome to bind thereto and/or initiate translation therefrom. Five different classes of ribozymes have been described based on their nucleotide sequence and/or three dimensional structure, namely, Tetrahymena group I intron, Rnase P, hammerhead ribozymes, hairpin ribozymes and hepatitis delta virus ribozymes. Generally, a ribozyme comprises a region of nucleotides (e.g., about 12 to 15 nucleotides) that are complementary to a target sequence, e.g., a nucleotide sequence set forth in any one of SEQ ID NOs: 1 to 11.

An RNAi (or siRNA or small interfering RNA) is a double stranded RNA molecule that is identical to a specific gene product. The dsRNA when expressed or introduced into a cell induces expression of a pathway that results in specific cleavage of a nucleic acid highly homologous to the dsRNA.

RNAi molecules are described, for example, by Fire et al., Nature 391: 806-811, 1998, and reviewed by Sharp, Genes & Development, 13: 139-141, 1999). As will be known to those skilled in the art, short hairpin RNA (“shRNA”) is similar to siRNA. However, the shRNA molecule comprises a single strand of nucleic acid with two complementary regions (highly homologous to the sequence of a region of an IRES or the complement thereof) separated by an intervening hairpin loop such that, following introduction to a cell, it is processed by cleavage of the hairpin loop into siRNA.

A preferred siRNA or shRNA molecule comprises a nucleotide sequence that is identical to about 19-21 contiguous nucleotides of the target mRNA (e.g., a nucleotide sequence set forth in any one of SEQ ID NOs: 1 to 11). Preferably, the target sequence commences with the dinucleotide AA, comprises a GC-content of about 30-70% (preferably, 30-60%, more preferably 40-60% and more preferably about 45%-55%), and is specific to the IRES of interest.

A nucleic acid compound may be directly introduced into a cell of the invention to perform a screening assay using, for example, a method described herein. Alternatively, a vector comprising the nucleic acid (e.g., an expression vector) is introduced using a method described herein.

6. Selection of a Compound that Inhibits IRES-Mediated Translation

In one embodiment, a cell of the invention is contacted with a test compound under conditions suitable for selection of reduced or inhibited IRES activity in the cell. Preferably, the cell is contacted with the compound for a time and under conditions sufficient for the compound to bind to the IRES and/or inhibit IRES-mediated translation prior to selection.

Suitable methods of selection will be apparent to the skilled artisan based on the description herein. By way of exemplification, the present inventors use an E. coli gpt gene linked to an IRES (in the exemplified form, an IRES from HCV). A cell expressing gpt is selected in the presence of, for example, aminopterin and mycophenolic acid and optionally, adenine and xanthine. This cell is then contacted with a test compound or a nucleic acid encoding the compound is introduced into the cell and the cell maintained for a time and under conditions sufficient for the compound to bind to the IRES and/or inhibit IRES-mediated translation. The cell is then selected by the addition of, for example, thioxanthine or 6-thioxanthine. Any cell capable of growing in the presence of thioxanthine is capable of reducing or preventing IRES-mediated translation, e.g., as a result of the compound reducing or preventing IRES activity.

Clearly, the method of selection used will depend upon the counter-selectable marker used. Suitable forms of selection will be apparent to the skilled artisan, for example, based on the description provided herein.

In one embodiment, the method of the invention comprises identifying a compound introduced into or contacted to the cell that is capable of inhibiting or reducing IRES-mediated translation. For example, this may be a direct form of identification wherein the cells used to perform the method of the invention are used in an array and the specific location of each compound tested in the array is recorded. By determining the location of each cell capable of reducing or inhibiting IRES-mediated translation, the compound is identified.

Suitable methods for arraying cells for such assays will be apparent to the skilled artisan and include, for example, the use of multi-well (e.g., 96 or 384 well) culture plates. Clearly, the present invention contemplates high-throughput methods, such as, for example, robotic mediated methods for analysing an array of cells and compounds.

In another embodiment, a compound that inhibits or reduces IRES-mediated translation is encoded by a nucleic acid introduced into the cell. To identify a compound capable of reducing or inhibiting IRES-mediated translation, the nucleic acid encoding the compound is isolated or amplified from the cell and analysed, for example, by sequencing.

For example, a cell capable of inhibiting or reducing IRES-mediated translation is selected using the method of the invention, the cell isolated and nucleic acid from the cell isolated. Nucleic acid encoding the compound that inhibits or reduces IRES-mediated translation is then amplified using PCR and the amplified nucleic acid sequenced. Methods for isolating nucleic acid from a cell, PCR and sequencing are known in the art and/or described, for example, in Ausubel et al (In: Current Protocols in Molecular Biology. Wiley Interscience, ISBN 047 150338, 1987) or Sambrook et al (In: Molecular Cloning: Molecular Cloning: A Laboratory Manual, Cold Spring Harbor Laboratories, New York, Third Edition 2001).

### 7. Confirmation of IRES Specific Inhibition

In one embodiment, the method of the invention additionally comprises determining a compound that selectively inhibits IRES-mediated translation rather than inhibiting cellular translation per se. A selective IRES inhibitor is determined or selected, for example, by performing a method comprising:
(i) expressing in a cell a first reporter gene operably linked to a promoter that is operable in the cell and a second reporter gene, wherein the second reporter gene is operably linked to the IRES;
(ii) contacting the cell with or introducing into the cell the compound under conditions sufficient for expression of the first and second reporter genes; and
(iii) selecting a cell in which the expression of the first reporter gene is not detectably reduced and the expression of the second reporter gene is reduced and said reduced expression is indicative of reduced IRES-mediated translation, thereby determining a compound that selectively reduces or inhibits IRES-mediated translation.

A suitable cell and methods for its production are described herein and are to be taken to apply mutatis mutandis to the present embodiment of the invention.

As the method of the invention detects the level of expression of a reporter gene, the reporter genes used in the method of the invention are preferably quantifiable. A suitable reporter gene will be apparent to the skilled artisan. For example, a reporter gene is a fluorescent reporter gene or an enzymatic reporter gene, e.g., as described herein.

Furthermore, it is preferred that the first and second reporter genes are different to thereby facilitate detection of each reporter gene individually.

Methods for determining the level of reporter gene expression will be apparent to the skilled artisan and will depend upon the type of reporter gene used. For example, in the case of a fluorescent reporter gene expression, as exemplified herein a sample is exposed to a light at a wavelength sufficient to excite the fluorescent reporter gene product and the level of fluorescence detected. Accordingly, in the case of two reporter genes that encode proteins that fluoresce at different wavelengths, the level of fluorescence is detected at one wavelength and then another.

For example, a cell is produced that comprises an expression construct comprising an eGFP gene operably connected to a promoter and a dsRED gene operably linked to an IRES. A test compound is contacted to or introduced into the cell and the level of eGFP expression is determined by exposing the cell to a light at 488 nm and the level of dsRED expression is determined by exposing the cell to light at 455 nm. Clearly, any fluorescent reporter gene is useful for performance of the invention, for example, a reporter gene described herein. In this regard it is preferable that the reporter gene coperably connected to a promoter and the reporter gene operably linked to an IRES fluoresce (i.e., emit light) at wavelengths that are sufficiently different to enable detection of each reporter gene independently (for example, there is little or no overlap in the emission spectra of the two reporter genes).

The use of fluorescent reporter genes is particularly preferred as the fluorescent gene products facilitate high-throughput analysis of cells to identify a compound that selectively inhibits IRES-mediated translation. For example, fluorescent activated cell sorting (FACS) analysis is performed to select a cell in which the expression of a reporter gene operably connected to a promoter is maintained at “normal” or “high” levels and in which the expression of a reporter gene operable linked to an IRES is reduced.

Clearly, an enzymatic reaction may also be used. For example, a cell expressing β-galactosidase operably connected to a promoter and dsRED operably linked to an IRES. Following contacting the cell with a test compound the level of dsRED expression is determined and then the level of β-galactosidase expression is determined by contacting the cell with x-gal and determining the level or amount of coloured precipitate formed.

As will be apparent from the foregoing, a degree of quantification is required to determine a change in the level of expression of the first or second reporter gene. Generally this level of quantification will be determined by comparing the level of reporter gene expression in a test sample to the level of reporter gene expression in a suitable control sample. Accordingly, in one embodiment, the method comprises:
(i) expressing in a cell a first reporter gene operably linked to a promoter that is operable in the cell and a second reporter gene, wherein the second reporter gene is operably linked to the IRES;
(ii) contacting the cell with or introducing into the cell the compound under conditions sufficient for expression of the first and second reporter genes;
(iii) determining the level of expression of the first reporter gene and the second reporter gene in the cell at (ii) and in a suitable control sample; and
(iv) selecting a cell in which the expression of the first reporter gene is not detectably reduced compared to the suitable control and the expression of the second reporter gene is reduced compared to the suitable control and said reduced expression is indicative of reduced IRES-mediated translation,
   thereby determining a compound that selectively reduces or inhibits IRES-mediated translation.

In one embodiment, the suitable control is the cell prior to contacting or introducing the test compound. Accordingly, the method comprises the additional step of determining the level of expression of the first and second reporter genes prior to contacting or introducing into the cell the compound (II).

In another embodiment, a suitable control is a cell that has not been contacted with a test compound. Preferably, the cell is substantially identical to the cell that is contacted with a test compound (i.e., is derived from the same cell and expresses each reporter gene at substantially the same level). Such a cell may provide a suitable control for a plurality of test assays, for example, a plurality of assays run in parallel.

In another embodiment, should the level of reporter gene expression be relatively consistent in the cell type used in the assay of the present embodiment, a suitable control need not be included in the assay. Rather, the average or mean level of expression of the first and/or second reporter gene is determined for a plurality of control samples and this value is used to determine a cell in which the level of expression of the first reporter gene is relatively unchanged and the level of expression of the second reporter gene is reduced.

To allow for a change in cell number, the present assay may comprise, for example, normalizing the level of each reporter gene relative to the number of cells used in the assay to thereby enable comparison between assays.

Methods for determining cell number are known in the art, and include, for example, manually counting the number of cells used in an assay, or, alternatively, counting a fraction of the number of cells used in an assay. For example, when using a microtitre plate, the number of cells in a fraction of the total area of the plate (e.g. 10% or 25% or 50%) of each well of the plate is counted, and this result used to estimate the number of cells in each well of the plate.

Alternatively, or in addition, a sample is normalized for cell number by detecting a protein that is expressed by the cells used in the assay. A protein useful in such an assay is one that is not affected by any conditions, e.g., compounds, to which the cells are exposed. For example, should the cells be exposed to various concentrations of a compound, a protein that is affected by the compound (i.e., the expression levels of the protein) is not useful for normalization. Various proteins useful for normalization are known in the art and include, for example, β-tubulin, actin, glyceraldehyde 3-phosphate dehydrogenase (GAPDH), β2 microglobulin, hydroxy-methylbilane synthase, hypoxanthine phosphoribosyl-transferase 1 (HPRT), ribosomal protein L13c, succinate dehydrogenase complex subunit A and TATA box binding protein (TBP). A suitable method for determining the level of expression of such a control protein will be apparent to the skilled person, for example, an immunoassay, such as, for example, an ELISA.

In another embodiment, the level of reporter gene expression is determined by comparing the level of expression detected to a standard curve. Methods for producing a standard curve will be apparent to the skilled artisan. Generally, a standard curve is produced by determining the level of, for example, fluorescence detected in the presence of a known amount of a fluorescent reporter gene. Accordingly, a plurality of known amounts of a fluorescent reporter gene product are assayed to determine the level of fluorescence produced and these levels plotted to produce a curve that is predictive of the absolute amount of fluorescent protein relative to fluorescence detected. The level of fluorescence detected in a sample is then compared to the standard curve to determine the amount of reporter gene product present.

### 8. IRES Inhibitory Compounds

In one embodiment, the present invention provides a compound identified or determined using the method of the invention.

In another embodiment, the present invention provides a method for providing a compound identified or determined using a method of the invention. For example, the present invention provides a process for determining a compound that inhibits or reduces IRES-mediated translation, said process comprising:
(i) performing a method described herein to determine a compound that inhibits or reduces IRES-mediated translation;
(ii) optionally, isolating the compound or a nucleic acid encoding the compound;
(iii) optionally, determining the structure of the compound; and
(iv) providing the compound or the name or structure of the compound.

Naturally, for compounds that are known albeit not previously tested for their function using a screen provided by the present invention, determination of the structure of the compound is implicit in step (i) supra. This is because the skilled artisan will be aware of the name and/or structure of the compound at the time of performing the screen.

As used herein, the term “providing the compound” shall be taken to include any chemical or recombinant synthetic means for producing said agent or alternatively, the provision of an agent that has been previously synthesized by any person or means. Suitable compounds and method for their production will be apparent to the skilled artisan and/or described herein.

In a preferred embodiment, the compound or the name or structure of the compound is provided with an indication as to its use e.g., as determined by a screen described herein.

In another example, the invention provides a process for providing a compound that inhibits or reduces IRES-mediated translation, said process comprising:
(i) performing a method described herein to determine a compound that inhibits or reduces IRES mediated translation;
(ii) optionally, isolating the compound or a nucleic acid encoding the compound;
(iii) optionally, determining the structure of the compound;
(iv) optionally, providing the name or structure of the compound; and
(v) providing, the compound.

### 9. Therapeutic Compounds

Anti-Viral Compounds

As several viruses use an IRES to induce Cap-independent translation of their genome or an RNA form of their genome, the present invention is preferably useful for determining a candidate anti-viral agent.

Accordingly, in one embodiment, the present invention provides a method for determining a candidate anti-viral compound, said method comprising:
(i) expressing in a cell a counter-selectable reporter gene wherein said counter selectable reporter gene is operably linked to a viral IRES;
(ii) contacting the cell with or introducing into the cell a candidate compound under conditions sufficient to kill or inhibit the growth of a cell expressing the counter-selectable reporter gene; and
(iii) selecting a cell in which the expression of the counter-selectable reporter gene is reduced and said reduced expression is indicative of reduced IRES-mediated translation,
   thereby determining a candidate anti-viral compound.

Both DNA and RNA viruses are known to use IRES to induce translation of mRNA from their genome. Accordingly, the method of the invention is useful for determining a candidate anti-DNA or anti-RNA viral compound.

As used herein the term “DNA virus” shall be taken to mean any form of virus that comprises DNA as its means for encoding polypeptides required for producing progeny particles. Preferably, the DNA virus comprises one or more IRES, for example, the DNA virus is a human herpes virus 8 (HHV8).

As used herein the term “RNA virus” shall be taken to mean any form of virus that comprises RNA as opposed to DNA as its means for encoding polypeptide/s required for producing progeny particles. A RNA virus may be merely a RNA molecule or alternatively a RNA molecule encompassed by an envelope that comprises one or more proteins. The term “RNA virus” includes a virus from a taxon selected from the group consisting of arenaviridae, bornaviridae, bunyaviridae, filoviridae, ophiovirus, orthomyxoviridae, paramyxoviridae, rhabdoviridae, tenuivirus, arteriviridae, astroviridae, bromoviridae, caliciviridae, closteroviridae, comoviridae, coronaviridae, flaviridae, furoviridae, luteoviridae, necrovirus, nodaviridae, picornaviridae, potexvirus, potyviridae, sequiviridae, sobemovirus, tetraviridae, tobamovirus, tobravirus, togaviridae, tombusviridae, tymovirus, birnaviridae, partitiviridae, reoviridae, totiviridae, hypoviridae and retroviridae. In a preferred embodiment, the RNA virus is a retrovirus.

As used herein, the term “retrovirus” shall be taken to mean a class of enveloped viruses that comprise genetic material in the form of RNA and use a reverse transcriptase to translate this RNA into DNA which is then incorporated into the genome of a host cell. A preferred retrovirus is from a genus selected from the group consisting of alpharetrovirus, betaretrovirus, gammaretrovirus, deltaretrovirus, epsilonretrovirus, lentivirus and spumavirus. For example, a suitable retrovirus is a hepatitis C virus (HCV) or a human immunodeficiency virus.

As RNA viruses and particularly, retroviruses, have a high mutation rate they are particularly suited to becoming resistant to current therapeutic strategies. However, as the structure of an IRES is important for its functioning and is well conserved between related viruses, a compound that inhibits IRES-mediated translation may provide an anti-viral compound that is not as sensitive to mutation as currently used strategies.

In a preferred embodiment, the compound has a large interaction interface for binding to an IRES and inhibiting its functioning. Such a compound is predicted to be less sensitive to point mutations and/or small structural changes in the structure of the IRES, as is observed in RNA-viruses. Accordingly, it is preferred that the candidate anti-viral agent is a peptide, preferably, a peptide that binds to an IRES with high affinity and inhibits IRES-mediated translation.

A suitable IRES, expression construct and cell is described supra and is to be taken to apply mutatis mutandis to the present embodiment of the invention.

The present invention additionally provides a process for manufacturing a medicament for the treatment of a viral infection or a disease associated therewith, said process comprising:
(i) determining a candidate antiviral compound using a method described herein
(ii) optionally, isolating the antiviral compound;
(c) optionally, providing the name or structure of the candidate antiviral compound;
(d) optionally, providing the candidate antiviral compound; and
(e) using the candidate antiviral compound in the manufacture of a medicament for the treatment of a viral infection or a disease associated therewith.

A disease associated with a viral infection shall be taken to mean a disease that is caused as a result of a viral infection and includes, for example, acquired autoimmune disease (AIDS) caused by HIV-1 infection, foot and mouth disease caused by foot and mouth disease virus infection, hepatitis C caused by hepatitis C virus infection and Karposi's sarcoma caused by HHV8 infection.

In this respect, the skilled artisan will be aware that a compound isolated by a method described herein according to any embodiment, or a peptide described herein according to any embodiment is useful for treating a viral disease. For example, the compound or peptide is useful for treating a disease caused by a virus comprising an IRES, such as, for example, a disease or disorder caused by a virus selected from the group consisting of poliovirus, Coxsackievirus, enterovirus, rhinovirus, hepatitis A virus, encephalomyocarditis virus, Theirler's encephalomyocarditis virus, foot and mouth disease virus, equine rhinitis virus, echovirus, cricket paralysis-like virus, bovine viral diarrhea virus, classical swine fever virus, hepatitis C virus, GB virus, SIV, HIV-1, human T-lymphotrophic virus, Moloney murine leukemia virus, Friend murine leukemia virus, Rous sarcoma virus, Karposi's sarcoma associate herpesvirus. Preferably, the disease or disorder is caused by hepatitis C virus. Preferably, the disease or disorder is caused by HIV.

In a preferred embodiment, the method of the present embodiment is used to manufacture a medicament for the treatment of a retroviral infection or a disease associated therewith.

Methods for manufacturing a medicament will be apparent to the skilled artisan. For example, the anti-viral compound may be formulated in a suitable excipient or diluent, such as, for example, an aqueous solvent, non-aqueous solvent, non-toxic excipient, such as a salt, preservative, buffer and the like. For example, a non-aqueous solvent is propylene glycol, polyethylene glycol, vegetable oil and injectable organic esters such as ethyloleate. Examples of a suitable aqueous solvent include water, alcoholic/aqueous solutions, saline solutions, parenteral vehicles such as sodium chloride, Ringer's dextrose, etc. Preservatives include antimicrobial, anti-oxidants, chelating agents and inert gases. The pH and exact concentration of the various components the formulation suitable for administration to the animal are adjusted according to routine skills in the art.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. Pharmaceutically compatible binding agents, and/or adjuvant materials can be included as part of the composition.

For administration by inhalation, the compounds are delivered in the form of an aerosol spray from pressured container or dispenser which contains a suitable propellant, e.g., a gas such as carbon dioxide, or a nebulizer.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Where the compound is a protein or peptide or antibody or fragment thereof, the agent can be administered via in vivo expression of the recombinant protein. In vivo expression can be accomplished via somatic cell expression according to suitable methods (see, e.g. U.S. Pat. No. 5,399,346). In this embodiment, nucleic acid encoding the protein can be incorporated into a retroviral, adenoviral or other suitable vector (preferably, a replication deficient infectious vector) for delivery (e.g., as described supra), or can be introduced into a transfected or transformed host cell capable of expressing the protein for delivery. In the latter embodiment, the cells can be implanted (alone or in a barrier device), injected or otherwise introduced in an amount effective to express the protein in a therapeutically effective amount.

The pH and exact concentration of the various components the formulation suitable for administration to the animal are adjusted according to routine skills in the art.

Following determination of an agent using a method described herein, the agent is additionally tested in vivo. For example, an anti-HCV compound is tested in an animal model of HCV. A suitable HCV model will be apparent to the skilled artisan and include, for example, chimpanzee (which may be directly infected with HCV or cDNA encoding same) (Kolykhalov et al. Science 277: 570-574, 1997). Alternatively, the tree shrew (Tupaia spp.) may also be directly infected with HCV as can a primary hepatocyte therefrom. Whole body irradiation of tree shrews has been shown to increase the efficiency of HCV infection (Xie et al., Virology 244: 513-520 1998).

Recently, a mouse model of HCV infection has also been developed, wherein primary hepatocytes in a matrix are transplanted under the renal capsule of immunodeficient nonobese diabetic (NOD)/SCID mice. The administration of an agonistic antibody against hepatocyte growth factor receptor (cMet) stimulated growth of the engrafted human hepatocytes. These mice are proposed to be susceptible to HCV infection (Ohashi et al., Nat. Med. 6(3): 327-331, 2000). By grafting hepatocytes into transgenic mice that expressed uPA in the liver from an albumin promoter (alb-uPA) and that are immunodeficient because of mutation of the recombination activation gene 2 (RAG-2) investigators have been able to replace up to 50% of the mouse liver hepatocytes with human hepatocytes. The human hepatocytes survive for as long as 35 weeks and support high levels of viremia following infection with HCV-infected serum. Injection of the serum from the viremic mouse to other mice engrafted with human hepatocytes resulted in serial transfer of HCV. Hepatitis C virus replication in mice with chimeric human livers (Mercer et al., Nat. Med. 7: 927-933 2001).

Alternatively, an anti-HIV-1 compound is tested, for example, using an HIV-1 infection model, such as, for example, the SHIV-macaque model or the HIV-1 chimpanzee model of HIV infection.

Anti-Cancer Compounds

As discussed herein some forms of cancer express one or more endogenous genes in a Cap-independent manner (e.g., c-myc or VEGF) and this expression may induce transformation of the cell or assist in cell survival despite chemotherapy. Such Cap-independent translation is induced by an IRES in the gene. Accordingly, suppression of such IRES-mediated translation may be useful for the treatment of some forms of cancer, or useful in combination with traditional chemotherapeutic approaches.

Accordingly, in one embodiment, the present invention provides a method for determining a candidate anti-cancer compound, said method comprising:
(i) expressing in a cell a counter-selectable reporter gene wherein said counter selectable reporter gene is operably linked to an IRES derived from a gene that is expressed in a Cap-independent manner in cancer;
(ii) contacting the cell with or introducing into the cell a candidate compound under conditions sufficient to kill or inhibit the growth of a cell expressing the counter-selectable reporter gene; and
(iii) selecting a cell in which the expression of the counter-selectable reporter gene is reduced and said reduced expression is indicative of reduced IRES-mediated translation,
   thereby determining a candidate anti-cancer compound.

By “cancer” is meant any disease or disorder characterized by hyperproliferation of a cell in a subject. The term cancer includes a primary cancer or tumor, a metastasis of a cancer or tumor, or a recurrence of a cancer or tumor. Preferably, the cancer is selected from the group consisting of prostate cancer, breast cancer, ovarian cancer, and Karposi's sarcoma.

A suitable IRES, expression construct and cell is described supra and is to be taken to apply mutatis mutandis to the present embodiment of the invention.

The present invention additionally provides a process for manufacturing a medicament for the treatment of a cancer, said process comprising:
(i) determining a candidate anti-cancer compound using a method described herein
(ii) optionally, isolating the anti-cancer compound;
(c) optionally, providing the name or structure of the candidate anti-cancer compound;
(d) optionally, providing the candidate anti-cancer compound; and
(e) using the candidate anti-cancer compound in the manufacture of a medicament for the treatment of a viral infection or a disease associated therewith.

As with an anti-viral compound, it is preferred that an anti-cancer compound is tested in a model of cancer. For example, a compound identified by the method of the invention is contacted to or introduced into a cancer cell line that expresses FGF-2 in a Cap-independent manner, such as, for example, a neuroblastoma cell line (e.g., SK-N-BE and SK-N-AS) or an osteosarcoma cell line (e.g., Saos2) and the effect of the peptide on FGF-2 expression and cell morphology/cell division determined.

Alternatively, a candidate anti-cancer compound is contacted to, administered to or introduced into a breast cancer cell line or a prostate cancer cell line, such as, for example, LnCAP, Du145, PC-3, T-47D, MCF-7, SW-13, MCF-12A or MCF-12F.

Preferably, the cell line is additionally treated with a chemotherapeutic agent (e.g., Gemcitabine; Eli Lilly) and/or γ radiation to thereby induce expression of, for example, XIAP. Cells are then monitored to determine cell death, for example, in response to the presence of a chemotherapeutic agent or exposure to γ radiation.

Furthermore, a candidate compound may be tested or analysed in vivo, for example, by administering the compound to a xenograft mouse model of prostate cancer (i.e., a mouse (usually a SCID mouse) in which a prostate cancer cell line or a prostate cancer cell from a subject is implanted (e.g., under the kidney capsule). Suitable methods for producing a xenograft model are known in the art and/or described, for example, in Wang et al., Prostate, 2005 (advance online publication). Preferably, the mouse model is additionally treated with, for example, a chemotherapeutic drug, to thereby induce expression, of XIAP. A compound that reduces or prevents tumor development (preferably, without adverse effect to the mouse) is considered a candidate agent for the treatment of cancer.

A candidate compound for the treatment of a cancer associated with Cap-independent translation of c-myc is validated, for example, in a cell or cell line derived from a subject suffering from multiple myeloma. Preferably, the cell comprises a mutation in the c-myc IRES that induces increased Cap-independent translation (e.g., as described, in Chappell et al., Oncogene, 19: 4437-4440, 2000). A compound that reduces or prevents Cap-independent expression of c-myc in such a cell represents a candidate compound for the treatment of cancer.

In one embodiment, the method of the invention provides for the use of a compound identified using a method described herein in the manufacture of a medicament for the treatment of a cancer. Suitable methods for producing a medicament are described supra and are to be taken to apply mutatis mutandis to the present embodiment of the invention.

In one embodiment, the medicament additionally comprises an additional chemotherapeutic compound. Accordingly, the medicament comprises a compound suitable to suppress or prevent expression of a gene (e.g. a gene that induces cell survival) by a Cap-independent manner and a compound that, for example, induces cell death.

### 10. IRES-Inhibitory Peptides

The present inventors have also clearly demonstrated the isolation of IRES-inhibitory peptides, e.g., a peptide comprising an amino acid sequence set forth in any one of SEQ ID NOs: 75 to 110. Alternatively, the present invention provides a peptide comprising an amino acid sequence consisting essentially of an amino acid sequence set forth in any one of SEQ ID NOs: 75 to 110. Alternatively, the present invention provides a peptide comprising an amino acid sequence consisting of an amino acid sequence set forth in any one of SEQ ID NOs: 75-110.

Accordingly, the present invention also provides an isolated or recombinant peptide identified by the method of the present invention or an analogue thereof.

Methods for producing peptides and/or peptide analogues will be apparent to the skilled artisan and/or described herein.

As used herein, the term “analogue” shall be taken to mean a peptide that is modified to comprise one or more naturally-occurring and/or non-naturally-occurring amino acids, provided that the peptide analogue displays IRES-inhibitory activity. For example, the term “analogue” encompasses peptide as described herein according to any embodiment comprising one or more conservative amino acid changes. The term “analogue” also encompasses a peptide comprising, for example, one or more D-amino acids. Such an analogue has the characteristic of, for example, reduced immunogenicity and/or protease resistance.

The term “analogue” shall also be taken to include a peptide that is derived from a peptide of the invention, e.g., a fragment or processed form of a peptide of the invention.

The term “analogue” also encompasses a derivatized peptide, such as, for example, a peptide modified to contain one or more-chemical moieties other than an amino acid.

The chemical moiety may be linked covalently to the peptide e.g., via an amino terminal amino acid residue, a carboxy terminal amino acid residue, or at an internal amino acid residue. Such modifications include the addition of a protective or capping group on a reactive moiety in the peptide, addition of a detectable label, and other changes that do not adversely destroy the activity of the peptide compound.

The present invention additionally provides a fusion protein comprising a peptide of the invention. For example, the fusion protein comprises a label, such as, for example, an epitope, e.g., a FLAG epitope or a V5 epitope or an HA epitope. Such a tag is useful for, for example, purifying the fusion protein. Additional suitable fusion proteins will be apparent to the skilled artisan based on the disclosure herein and include, for example, a fusion protein comprising a plurality of the peptides described herein according to any embodiment.

Alternatively, or in addition, the fusion protein comprises a protein transduction domain, e.g., as described herein, e.g., a HIV-1 TAT protein transduction domain. Optionally, the protein transduction domain is separated from the peptide of the invention, for example, by a linker.

Peptide Analogues

Suitable peptide analogues include, for example, a peptide comprising one or more conservative amino acid substitutions. A “conservative amino acid substitution” is one in which the amino acid residue is replaced with an amino acid residue having a similar side chain.

Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), .beta.-branched side chains (e.g., threonine, valine, isoleucine) and aromatic side chains (e.g., tyrosine, phenylalanine, tryptophan, histidine).

Analogues of the peptides of the invention are intended to include compounds in which one or more amino acids of the peptide structure are substituted with a homologous amino acid such that the properties of the original modulator are maintained. Preferably conservative amino acid substitutions are made at one or more amino acid residues.

The importance of the hydropathic amino acid index in conferring interactive biological function on a protein is generally understood in the art (Kyte & Doolittle, J. Mol. Biol. 157, 105-132, 1982). It is known that certain amino acids may be substituted for other amino acids having a similar hydropathic index or score and still retain a similar biological activity. The hydropathic index of amino acids also may be considered in determining a conservative substitution that produces a functionally equivalent molecule. Each amino acid has been assigned a hydropathic index on the basis of their hydrophobicity and charge characteristics, as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making changes based upon the hydropathic index, the substitution of amino acids whose hydropathic indices are within +/−0.2 is preferred. More preferably, the substitution will involve amino acids having hydropathic indices within +/−0.1, and more preferably within about +/−0.05.

It is also understood in the art that the substitution of like amino acids is made effectively on the basis of hydrophilicity. As detailed in U.S. Pat. No. 4,554,101, the following hydrophilicity values have been assigned to amino acid residues: arginine (+3.0); lysine (+3.0); aspartate (+3.0+/−0.1); glutamate (+3.0+/−0.1); serine (+0.3); asparagine (+0.2); glutamine (+0.2); glycine (0); threonine (−0.4); proline (−0.5+/−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, it is preferred to substitute amino acids having hydrophilicity values within about +/−0.2 of each other, more preferably within about +/−0.1, and even more preferably within about +/−0.05

Additional preferred peptide analogues have reduced immunogenicity compared to a peptide of the invention. Alternatively, or in addition, a preferred peptide analogue has enhanced stability compared to a peptide of the invention.

It also is contemplated that sterically similar compounds may be formulated to mimic the key portions of the peptide structure. Such compounds, which may be termed peptidomimetics, may be used in the same manner as the peptides of the invention and hence are also analogues of a peptide of the invention. The generation of such an analogue may be achieved by the techniques of modeling and chemical design known to those of skill in the art. It will be understood that all such sterically similar peptide analogues fall within the scope of the present invention.

Another method for determining the “equivalence” of modified peptides involves a functional approach. For example, a given peptide analogue is tested for its IRES inhibitory activity e.g., using any screening method described herein.

Particularly preferred analogues of a peptide of the invention will comprise one or more non-naturally occurring amino acids or amino acid analogues. For example, the peptide of the invention may comprise one or more naturally occurring non-genetically encoded L-amino acids, synthetic L-amino acids or D-enantiomers of an amino acid. For example, the peptide comprises only D-amino acids. More particularly, the analogue may comprise one or more residues selected from the group consisting of: hydroxyproline, β-alanine, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylananine 3-benzothienyl alanine 4-chlorophenylalanine, 2-fluorophenylalanine, 3-fluorophenylalanine, 4-fluorophenylalanine, penicillamine, 1,2,3,4-tetrahydro-tic isoquinoline-3-carboxylic acid β-2-thienylalanine, methionine sulfoxide, homoarginine, N-acetyl lysine, 2,4-diamino butyric acid, ρ-aminophenylalanine, N-methylvaline, homocysteine, homoserine, 8-amino hexanoic acid, 6-amino valeric acid, 2,3-diaminobutyric acid and mixtures thereof.

Commonly-encountered amino acids that are not genetically encoded and which can be present, or substituted for an amino acid in an analogue of a peptide of the invention include, but are not limited to, β-alanine (β-Ala) and other omega-amino acids such as 3-aminopropionic acid (Dap), 2,3-diaminopropionic acid (Dpr), 4-aminobutyric acid and so forth; α-aminoisobutyric acid (Aib); ε-aminohexanoic acid (Aha); δ-aminovalericacid (Ava); methylglycine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-NaI); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); .beta.-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH₂)); N-methyl valine (MeVal); homocysteine (hCys) and homoserine (hSer).

Other amino acid residues that are useful for making the peptides and peptide analogues described herein can be found, e.g., in Fasman, 1989, CRC Practical Handbook of Biochemistry and Molecular Biology, CRC Press, Inc., and the references cited therein.

The present invention additionally encompasses an isostere of a peptide described herein. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) known to those skilled in the art. Such modifications include modifications of the amide nitrogen, the α-carbon, amide carbonyl, complete replacement of the amide bond, extensions, deletions or backbone crosslinks. Several peptide backbone modifications are known, including ψ[CH₂S], ψ[CH₂NH], ψ[CSNH₂], ψ[NHCO], ψ[COCH₂], and ψ[(E) or (Z) CH═CH]. In the nomenclature used above, v indicates the absence of an amide bond. The structure that replaces the amide group is specified within the brackets.

Other modifications include, for example, an N-allyl (or aryl) substitution (ψ[CONR]), or backbone crosslinking to construct lactams and other cyclic structures. Other derivatives of the modulator compounds of the invention include C-terminal hydroxymethyl derivatives, O-modified derivatives (e.g., C-terminal hydroxymethyl benzyl ether), N-terminally modified derivatives including substituted amides such as alkylamides and hydrazides.

In another embodiment, the peptide analogue is a retro peptide analogue (Goodman et al., Accounts of Chemical Research, 12: 1-7, 1979). A retro peptide analogue comprises a reversed amino acid sequence of a peptide of the present invention. For example, a retro peptide analogue of a peptide of the present invention comprises an amino acid sequence set forth in any one of SEQ ID NOs: 75 to 102, 104 or 106 in which the amino acid sequence of two or more amino acids in the peptide is reversed. Preferably, the retro peptide analogue of a peptide of the present invention comprises an amino acid sequence set forth in any one of SEQ ID NOs: 75 to 102, 104 or 106 in which the sequence of each of the residues in said peptide is reversed.

In another embodiment, a retro-peptide analogue of a peptide of the present invention consists of an amino acid sequence set forth in any one of SEQ ID NOs: 103, 105, 107, 108, 109 or 110 in which the amino acid sequence of two or more amino acids in the peptide is reversed. Preferably, the retro peptide analogue of a peptide of the present invention consists of an amino acid sequence set forth in any one of SEQ ID NOs: 103, 105, 107, 108, 109 or 110 in which the sequence of each of the residues in said peptide is reversed.

In a preferred embodiment, an analogue of a peptide of the invention is a retro-inverted peptide analogue (see, for example, Sela and Zisman, FASEB J. 11:449, 1997). Evolution has ensured the almost exclusive occurrence of L-amino acids in naturally occurring proteins. As a consequence, virtually all proteases cleave peptide bonds between adjacent L-amino acids. Accordingly, artificial proteins or peptides composed of D-amino acids are preferably resistant to proteolytic breakdown. Retro-inverted peptide analogues are isomers of linear peptides in which the direction of at least a portion and preferably the entire amino acid sequence is reversed (retro) and the chirality, D- or L-, of one or more amino acids therein is inverted e.g., using D-amino acids rather than L-amino acids, e.g., Jameson et al., Nature, 368, 744-746 (1994); Brady et al., Nature, 368, 692-693 (1994). The net result of combining D-enantiomers and reverse synthesis is that the positions of carbonyl and amino groups in each amide bond are exchanged, while the position of the side-chain groups at each alpha carbon is preserved.

An advantage of retro-inverted peptide analogues is their enhanced activity in vivo due to improved resistance to proteolytic degradation, i.e., the peptide has enhanced stability. (e.g., Chorev et al., Trends Biotech. 13, 438-445, 1995).

Retro-inverted peptide analogues may be complete or partial. Complete retro-inverted peptide analogues are those in which a complete sequence of a peptide of the invention is reversed and the chirality of each amino acid in a sequence is inverted. Accordingly, a retro-inverted peptide analogue of a peptide of the present invention comprises an amino acid sequence set forth in any one of SEQ ID NOs: 75 to 102, 104 or 106 in which the sequence of each of the residues in said peptide is reversed and the chirality of each amino acid in the sequence is reversed. For example, a retro-inverted peptide analogue of a peptide of the present invention comprises an amino acid sequence set forth in any one of SEQ ID NOs: 75 to 102, 104 or 106 in which the sequence of each of the residues in said peptide is reversed and each amino acid is a D-amino acid.

Alternatively, a retro-inverted peptide analogue of a peptide of the present invention consists of an amino acid sequence set forth in any one of SEQ ID NOs: 103, 105, 107, 108, 109 or 110 in which the sequence of each of the residues in said peptide is reversed and the chirality of each amino acid in the sequence is reversed. Preferably, the retro-inverted peptide analogue of a peptide of the present invention consists of an amino acid sequence set forth in any one of SEQ ID NOs: 103, 105, 107, 108, 109 or 110 in which the sequence of each of the residues in said peptide is reversed and each amino acid is a D-amino acid.

Partial retro-inverso peptide analogues are those in which only some of the peptide bonds are reversed and the chirality of only those amino acid residues in the reversed portion is inverted. For example, one or two or three or four or five or six or seven or eight or nine or ten or eleven or twelve or thirteen or fourteen or fifteen or sixteen or seventeen or eighteen or nineteen or twenty or twenty one or twenty two or twenty three or twenty four or twenty five or twenty six or twenty seven or twenty eight or twenty nine or thirty or thirty one or thirty two or thirty three or thirty four or thirty five or thirty six or thirty seven or thirty eight or thirty nine or forty or forty one or forty two or forty three or forty four or forty five amino acid residues are D-amino acids. The present invention clearly encompasses both partial and complete retro-inverted peptide analogues.

For example, the description herein above of retro-inverted peptide analogues shall be taken to apply mutatis mutandis to partial retro-inverted analogues.

Preferred partial retro-inverted peptide analogues are those in which the chirality of each amino acid in the sequence of the peptide other than glycine is reversed. For example, each amino acid residue in the sequence of the retro-invented peptide analogue other than glycine is a D-amino acid.

In another embodiment, an analogue of a peptide is modified to reduce the immunogenicity of said analogue. Such reduced immunogenicity is useful for a peptide that is to be injected into a subject. Methods for reducing the immunogenicity of a peptide will be apparent to the skilled artisan. For example, an antigenic region of a peptide is predicted using a method known in the art and described, for example, in Kolaskar and Tongaonkar FEBS Letters, 276:172-174, 1990. Any identified antigenic region may then be modified to reduce the immunogenicity of a peptide analogue, provided that said analogue is an IRES-inhibitory peptide analogue.

Alternatively, or in addition, Tangri et al., The Journal of Immunology, 174: 3187-3196, 2005, describe a process for identifying an antigenic site in a peptide and modifying said site to thereby reduce the immunogenicity of the peptide without significantly reducing the activity of said protein. The approach is based on 1) the identification of immunodominant epitopes, e.g., by determining binding to purified HLA molecules; and 2) reducing their binding affinity to HLA-DR molecules to levels below those associated with naturally occurring helper T lymphocyte epitopes. Generally, the approach is based on quantitative determination of HLA-DR binding affinity coupled with confirmation of these epitopes by in vitro immunogenicity testing.

Other preferred peptide analogues include, for example, a fragment or processed form of a peptide of the invention. For example, an antigenic determinant is deleted from a peptide of the invention thereby producing an analogue having reduced immunogenicity.

Alternatively, or in addition, a cleavage site of a protease active in a subject to which the peptide is to be administered is mutated and/or deleted to produce a stable analogue of a peptide of the invention.

Methods for producing additional analogues of a peptide of the invention will be apparent to the skilled artisan and include recombinant methods. For example, a nucleic acid encoding a peptide of the invention or an analogue thereof is amplified using mutagenic PCR and the resulting nucleic acid expressed to produce a peptide using a method known in the art and/or described herein.

For example, the nucleic acid fragments are modified by amplifying a nucleic acid fragment using mutagenic PCR. Such methods include a process selected from the group consisting of: (i) performing the PCR reaction in the presence of manganese; and (ii) performing the PCR in the presence of a concentration of dNTPs sufficient to result in misincorporation of nucleotides.

Methods of inducing random mutations using PCR are known in the art and are described, for example, in Dieffenbach (ed) and Dveksler (ed) (In: PCR Primer: A Laboratory Manual, Cold Spring Harbour Laboratories, NY, 1995). Furthermore, commercially available kits for use in mutagenic PCR are obtainable, such as, for example, the Diversify PCR Random Mutagenesis Kit (Clontech) or the GeneMorph Random Mutagenesis Kit (Stratagene).

In one embodiment, PCR reactions are performed in the presence of at least about 200 μM manganese or a salt thereof, more preferably at least about 300 μM manganese or a salt thereof, or even more preferably at least about 500 μM or at least about 600 μM manganese or a salt thereof. Such concentrations manganese ion or a manganese salt induce from about 2 mutations per 1000 base pairs (bp) to about 10 mutations every 1000 bp of amplified nucleic acid (Leung et al Technique 1, 11-15, 1989).

In another embodiment, PCR reactions are performed in the presence of an elevated or increased or high concentration of dGTP. It is preferred that the concentration of dGTP is at least about 25 μM, or more preferably between about 50 μM and about 100 μM. Even more preferably the concentration of dGTP is between about 100 μM and about 150 μM, and still more preferably between about 150 μM and about 200 μM. Such high concentrations of dGTP result in the misincorporation of nucleotides into PCR products at a rate of between about 1 nucleotide and about 3 nucleotides every 1000 bp of amplified nucleic acid (Shafkhani et al BioTechniques 23, 304-306, 1997).

Alternatively, an analogue of a peptide of the invention is produced by performing site-directed mutagenesis. Suitable methods of site-directed mutagenesis are known in the art and/or described in Dieffenbach (ed) and Dveksler (ed) (In: PCR Primer: A Laboratory Manual, Cold Spring Harbour Laboratories, NY, 1995).

Peptide analogues of the present invention also encompass a peptide of the invention or an analogue thereof as described herein in any embodiment that is modified to contain one or more-chemical moieties other than an amino acid. The chemical moiety may be linked covalently to the peptide or analogue e.g., via an amino terminal amino acid residue, a carboxy terminal amino acid residue, or at an internal amino acid residue. Such modifications include the addition of a protective or capping group on a reactive moiety in the peptide, addition of a detectable label, and other changes that do not adversely destroy the activity of the peptide compound (e.g., the IRES inhibitory activity of the peptide).

An “amino terminal capping group” of a peptide described herein is any chemical compound or moiety that is covalently linked or conjugated to the amino terminal amino acid residue of a peptide or analogue. An amino-terminal capping group may be useful to inhibit or prevent intramolecular cyclization or intermolecular polymerization, to protect the amino terminus from an undesirable reaction with other molecules, or to provide a combination of these properties. A peptide compound of this invention that possesses an amino terminal capping group may possess other beneficial activities as compared with the uncapped peptide, such as enhanced efficacy or reduced side effects. Examples of amino terminal capping groups that are useful in preparing peptide derivatives according to the invention include, but are not limited to, 1 to 6 naturally occurring L-amino acid residues, preferably, 1-6 lysine residues, 1-6 arginine residues, or a combination of lysine and arginine residues; urethanes; urea compounds; lipoic acid (“Lip”); glucose-3-O-glycolic acid moiety (“Gga”); or an acyl group that is covalently linked to the amino terminal amino acid residue of a peptide, wherein such acyl groups useful in the compositions of the invention may have a carbonyl group and a hydrocarbon chain that ranges from one carbon atom (e.g., as in an acetyl moiety) to up to 25 carbons (e.g., palmitoyl group, “Palm” (16:0) and docosahexaenoyl group, “DHA” (C22:6-3)). Furthermore, the carbon chain of the acyl group may be saturated, as in Palm, or unsaturated, as in DHA. It is understood that when an acid, such as docosahexaenoic acid, palmitic acid, or lipoic acid is designated as an amino terminal capping group, the resultant peptide compound is the condensed product of the uncapped peptide and the acid.

A “carboxy terminal capping group” of a peptide compound described herein is any chemical compound or moiety that is covalently linked or conjugated to the carboxy terminal amino acid residue of the peptide compound. The primary purpose of such a carboxy terminal capping group is to inhibit or prevent intramolecular cyclization or intermolecular polymerization, to promote transport of the peptide compound across the blood-brain barrier, and to provide a combination of these properties. A peptide of this invention possessing a carboxy terminal capping group may also possess other beneficial activities as compared with the uncapped peptide, such as enhanced efficacy, reduced side effects, enhanced hydrophilicity or enhanced hydrophobicity. Carboxy terminal capping groups that are particularly useful in the peptides described herein according to any embodiment include primary or secondary amines that are linked by an amide bond to the α-carboxyl group of the carboxy terminal amino acid of the peptide compound. Other carboxy terminal capping groups useful in the invention include aliphatic primary and secondary alcohols and aromatic phenolic derivatives, including flavenoids, with 1 to 26 carbon atoms, which form esters when linked to the carboxylic acid group of the carboxy terminal amino acid residue of a peptide compound described herein.

Other chemical modifications of a peptide or analogue, include, for example, glycosylation, acetylation (including N-terminal acetylation), carboxylation, carbonylation, phosphorylation, PEGylation, amidation, addition of trans olefin, substitution of α-hydrogens with methyl groups, derivatization by known protecting/blocking groups, circularization, inhibition of proteolytic cleavage (e.g., using D amino acids), linkage to an antibody molecule or other cellular ligand, etc. Any of numerous chemical modifications may be carried out by known techniques, including but not limited to specific chemical cleavage by cyanogen bromide, trypsin, chymotrypsin, papain, V8 protease, NaBH₄, acetylation, formylation, oxidation, reduction, etc.

Linkers

Each of the components of an analogue of a peptide of the invention or fusion protein comprising a peptide of the invention may optionally be separated by a linker that facilitates the independent folding of each of said components. A suitable linker will be apparent to the skilled artisan. For example, it is often unfavourable to have a linker sequence with high propensity to adopt α-helix or β-strand structures, which could limit the flexibility of the protein and consequently its functional activity. Rather, a more desirable linker is a sequence with a preference to adopt an extended conformation. In practice, most currently designed linker sequences have a high content of glycine residues that force the linker to adopt loop conformation. Glycine is generally used in designed linkers because the absence of a β-carbon permits the polypeptide backbone to access dihedral angles that are energetically forbidden for other amino acids.

Preferably, the linker is hydrophilic, i.e. the residues in the linker are hydrophilic.

Linkers comprising glycine and/or serine have a high freedom degree for linking of two proteins, i.e., they enable the fused proteins to fold and produce functional proteins. Robinson and Sauer Proc. Natl. Acad. Sci. 95: 5929-5934, 1998 found that it is the composition of a linker peptide that is important for stability and folding of a fusion protein rather than a specific sequence. For example, the authors found that a fusion protein comprising a linker consisting almost entirely of glycine was unstable. Accordingly, the use of amino acid residues other than glycine, such as, for example, alanine or serine, is also useful for the production of a linker.

In one embodiment, the linker is a glycine rich linker. Preferably, the linker is a glycine linker that additionally comprises alanine and/or serine.

### 11. Compositions

Preferably, a peptide and/or fusion protein and/or analogue of the present invention is provided in a composition, e.g., a pharmaceutical composition. Such a composition additionally comprises, for example, a suitable carrier, e.g., pharmaceutically acceptable carrier. The term “carrier” as used herein, refers to a carrier that is conventionally used in the art to facilitate the storage, administration, and/or the biological activity of an active agent, i.e., an IRES-inhibitor peptide described herein according to any embodiment. A carrier may also reduce any undesirable side effects of the active agent. A suitable carrier is stable, i.e., incapable of reacting with other ingredients in the formulation. The carrier does not produce significant local or systemic adverse effect in recipients at the dosages and concentrations employed for treatment. Such carriers are generally known in the art. Suitable carriers for this invention include those conventionally used. Water, saline, aqueous dextrose, and glycols are preferred liquid carriers, particularly (when isotonic) for solutions. Alternatively, the carrier is selected from various oils, including those of petroleum, animal, vegetable or synthetic origin, for example, peanut oil, soybean oil, mineral oil, sesame oil, and the like. Suitable pharmaceutical carriers include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like.

A composition comprising a peptide of the invention or a derivative or analogue thereof can be subjected to conventional pharmaceutical expedients, such as sterilization, and can contain conventional pharmaceutical additives, such as preservatives, stabilizing agents, wetting, or emulsifying agents, salts for adjusting osmotic pressure, buffers, and the like. Other acceptable components in the composition of the invention include, but are not limited to, isotonicity-modifying agents such as water, saline, and buffers including phosphate, citrate, succinate, acetic acid, and other organic acids or their salts.

Preferably, a composition of the invention also includes one or more stabilizers, reducing agents, anti-oxidants and/or anti-oxidant chelating agents. The use of buffers, stabilizers, reducing agents, anti-oxidants and chelating agents in the preparation of protein-based compositions, is known in the art and described, for example, in Wang et al. J. Parent. Drug Assn. 34:452-462, 1980; Wang et al. J. Parent. Sci. Tech. 42:S4-S26 (Supplement), 1988. Suitable buffers include acetate, adipate, benzoate, citrate, lactate, maleate, phosphate, tartarate, borate, tri(hydroxymethyl aminomethane), succinate, glycine, histidine, the salts of various amino acids, or the like, or combinations thereof. Suitable salts and isotonicifiers include sodium chloride, dextrose, mannitol, sucrose, trehalose, or the like. Where the carrier is a liquid, it is preferred that the carrier is hypotonic or isotonic with oral, conjunctival, or dermal fluids and has a pH within the range of 4.5-8.5. Where the carrier is in powdered form, it is preferred that the carrier is also within an acceptable non-toxic pH range.

In some embodiments, the peptide of the invention or analogue thereof is incorporated within a composition for administration to a mucus membrane, e.g., by nasal administration. Such a composition generally includes a biocompatible polymer functioning as a carrier or base. Such polymer carriers include polymeric powders, matrices or microparticulate delivery vehicles, among other polymer forms. The polymer can be of plant, animal, or synthetic origin. Often the polymer is crosslinked. Additionally, in these delivery systems the peptide or analogue can be functionalized in a manner where it can be covalently bound to the polymer and rendered inseparable from the polymer by simple washing. Polymers useful in this respect are desirably water interactive and/or hydrophilic in nature to absorb significant quantities of water, and they often form hydrogels when placed in contact with water or aqueous media for a period of time sufficient to reach equilibrium with water.

Drug delivery systems based on biodegradable polymers are preferred in many biomedical applications because such systems are broken down either by hydrolysis or by enzymatic reaction into non-toxic molecules. The rate of degradation is controlled by manipulating the composition of the biodegradable polymer matrix. These types of systems can therefore be employed in certain settings for long-term release of biologically active agents. Examples of suitable biodegradable polymers include, for example, poly(glycolic acid) (PGA), poly-(lactic acid) (PLA), and poly(D,L-lactic-co-glycolic acid) (PLGA).

Alternatively, a peptide or analogue thereof of the invention can be administered via in vivo expression of the recombinant protein. In vivo expression can be accomplished via somatic cell expression according to suitable methods (see, e.g. U.S. Pat. No. 5,399,346). In this embodiment, nucleic acid encoding the protein is incorporated into a retroviral, adenoviral or other suitable vector (preferably, a replication deficient infectious vector) for delivery, or can be introduced into a transfected or transformed host cell capable of expressing the protein for delivery. In the latter embodiment, the cells are implanted (alone or in a barrier device), injected or otherwise introduced in an amount effective to express the protein in a therapeutically effective amount.

Preferred compositions comprise an IRES-inhibitory peptide as described herein in accordance with any embodiment, and an antiviral agent. Such compositions are useful for the treatment or prevention of viral infection, particularly an infection by a virus that makes use of an IRES, e.g., for replication. The skilled artisan will be aware of suitable antiviral agents. For example, the antiviral agent is a nucleoside analogue, such as, for example, Zidovudine pyrimidine, Acyclovir purine analogue, Ganciclovir purine analog, Vidarabine purine analogue, Idoxuridine pyrimidine Trifluridine pyrimidine, Foscamet, tricyclic amantadine, Rimantadine, Amantadine, Ribavirin purine analog, Didanosine purine analog and Zalcitabine pyrimidine.

Alternatively, or in addition, the antiviral agent is a protease inhibitor, such as, for example an oligopeptide analog, such as saquinavir (Roche Laboratories), indinavir (MercK) or ritonavir (Abbott Laboratories), which are fully described in detail in U.S. Pat. Nos. 5,413,999 and 5,476,874.

### 11. Methods of Administration or Application

In the case of administration to an animal or a human, numerous methods of administering an effective amount of an isolated peptide of the present invention or an analogue thereof are available for use by the skilled artisan. For example, the peptide or analogue is introduced topically (e.g., in the form of a cream or a spray or a powder), parenterally, transmucosally, e.g., orally, nasally, or rectally, or transdermally, intra-arteriolely, intramuscularly, intradermally, subcutaneously, intraperitoneally, intraventricularly, and intracranially. Such administration can also occur via bolus administration. Oral solid dosage forms are described generally in Remington's Pharmaceutical Sciences, 18th Ed. 1990 (Mack Publishing Co. Easton Pa. 18042) at Chapter 89, and; Marshall, K. In: Modern Pharmaceutics Edited by G. S. Banker and C. T. Rhodes Chapter 10, 1979). A method and composition for pulmonary delivery of drugs for systemic effect is described in U.S. Pat. No. 5,451,569. Systems of aerosol delivery, such as the pressurized metered dose inhaler and the dry powder inhaler are disclosed in Newman, S. P., Aerosols and the Lung, Clarke, S. W. and Davia, D. editors, pp. 197-22 and can be used in connection with the present invention.

In another embodiment, the peptide of the present invention or analogue thereof, is delivered in a vesicle, in particular a liposome (see Langer, Science 249:1527-1533 (1990); Treat et al., in Liposomes in the Therapy of infectious Disease and Cancer,

The present invention is described further with reference to the following non-limiting examples.

### EXAMPLE 1

### An Assay for Determining Inhibitors of HCV IRES-Mediated Translation

### 1.1 Vector Construction

A vector comprising the fluorescent marker dsRED2 the expression of which is operably under the control of the CMV promoter and the E. coli gpt gene operably linked to a HCV IRES comprising the nucleotide sequence set forth in SEQ ID NO: 6 was produced to determine an inhibitor of HCV IRES-mediated translation.

Nucleic acids encoding dsRED2 was excised from the commercially available vector dsREDII (Clontech) and cloned into the multiple cloning site of the pcDNA3 expression vector (Invitrogen). The HCV IRES was amplified using PCR from a plasmid and cloned downstream of the dsRED2 encoding nucleic acid. The E. coli gpt gene lacking an ATG start codon was cloned in-frame and downstream of the HCV IRES and males use of the ATG start codon in the IRES.

The resulting vector, designated pcDNA3-red-HCVIRES-gpt, was then sequenced in both the 5′ and 3′ directions to ensure the correct nucleotide sequence of each of the inserted fragments.

### 1.2 Cell Transfection

Stable monoclonal HEK293 cells comprising the pcDNA3-red-HCVIRES-gpt vector were produced using the serial dilution method. Briefly, cells were transfected with Lipofectamine™ (Invitrogen Corporation) essentially according to manufacturer's instructions. Following a sufficient time to permit the selectable marker gene to be expressed, e.g., about 16 hours, cells were maintained in the presence of a suitable antibiotic, e.g., G418. Cells were maintained in the presence of the antibiotic for a sufficient time to ensure that only transfected cells survived, e.g., about 7-10 days. Surviving cells were then trypsinized and serial dilutions of the resulting solution re-plated and maintained in media containing a suitable antibiotic until the formation of monoclonal colonies, e.g., about 2 weeks. Cells from the resulting colonies are then screened or frozen for later use.

1.3 Identification of Cells Expressing dsRED and gpt

Cells were then screened to identify those capable of expressing dsRED2 using fluorescence using standard methods in the art.

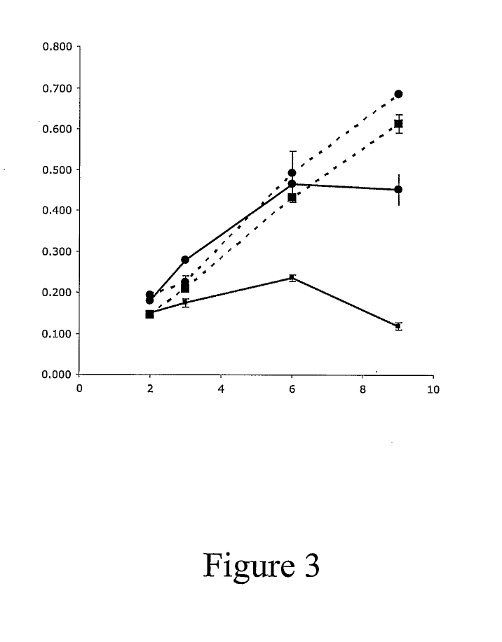

Cells were also screened to determine those capable of expressing gpt under the control of HCV IRES. Briefly, transfected cells or control cells were maintained either in the absence or presence of 6-thioxanthine (100 μM) for up to 9 days. To determine any change in cell number an optical density measurement was performed at 590 nm at days 2, 6 and 9 after the addition of 6-thioxanthine to the relevant cells. As shown in FIG. 3, cells comprising the pcDNA3-red-HCVIRES-gpt vector were considerably more susceptible to 6-thioxanthine selection than control cells.

### EXAMPLE 2

### Identifying Peptides Inhibitors of HCV IRES-Mediated Translation

A library of nucleic acid fragments from biodiverse gene fragments cloned into the expression vector pYTB3 (Phylogica Limited, Perth, Australia) was produced essentially as described in published International Application No. PCT/AU2004/000214. The nucleic acid fragments in the vector were amplified by PCR using a first primer capable of hybridizing to the sequence encoding the FLAG tag in the vector and a second capable of hybridizing to a sequence in the vector 3′ to the insertion site of the nucleic acid fragment. The resulting amplicons were then cloned into the pcDNA3 vector (invitrogen Corporation) to produce pcDNA3-peptide.

The resulting library is estimated to comprise approximately 1 million different nucleic acid fragments.

To identify peptide inhibitors of HCV-mediated translation, the cells described in Example 1.3 were transiently transfected with the pcDNA3-peptide vector using lipofectamine (Invitrogen Corporation). Cells were maintained in standard media for 24 hours to allow for peptide expression, after which they were transferred to selective media comprising 100 μM 6-thioxanthine. Cells were maintained in selective media for about 6 days with daily media changes.

After 6 days of selection, the majority of cells were dead. Cells were washed with phosphate buffered saline (PBS) to remove dead cells, and live cells isolated by trypsinization.

RNA was then isolated from the isolated cells using TRIZOL (Invitrogen Corporation) and nucleic acid encoding the peptide expressed by the cell amplified using reverse transcriptase-mediated PCR.

Amplified nucleic acids were cloned into the pcDNA3 vector and transformed into bacterial cells. Nucleic acid from individual colonies was then isolated and sequenced using standard methods. The nucleic acid sequences of the isolated nucleic acid fragments are set forth in Table 1.

TABLE 1

Amino acid sequences of peptides identified in screen to
identify inhibitors of IRES-mediated translation.

| | | | | |
|---|---|---|---|---|
| Peptide | FLAG + Peptide amino acid | SEQ ID | Peptide amino acid | SEQ ID |
| ID | sequence | NO: | Sequence | NO: |

| | | | | |
|---|---|---|---|---|
| IP 1-01 | RSDYKDVDDKAYQLMQRY | 75 | AYQLMQRYRY* | 89 |
| | RY* | | | |
| IP 1-02 | RSDYKDDDDKAYQSIIEVPI | 76 | AYQSIIEVIPIDASIPVL | 90 |
| | DASIPVLVGPHMPGRTAAA | | VGPHMPGRTAAARA | |
| | RACI* | | CI* | |
| IP 1-04 | RSDYKDDDDKVYQSMRRC | 77 | VYQSMRRCRVPIDASI | 91 |
| | RVPIDASIPVLVGPHMPGRT | | PVLVGPHMPGRTAAA | |
| | AAARACI* | | RACI* | |
| IP 1-06 | RSDYKDDDDKVVVSRKGY | 78 | VVVSRKGYQLMHRY | 92 |
| | QLMHRYRY* | | RY* | |
| IP 1-09 | RSDYKDDDDKVVDRKGTN | 79 | VVDRKGTN* | 93 |
| | * | | | |
| IP 1-17 | RSDYKDDDDTIDASIPVLVG | 80 | TIDASIPVLVGPHMPG | 94 |
| | PHMPGRTAAARACI* | | RTAAARACI* | |
| IP 1-18 | RSDYKDDDDKAYQSITVPID | 81 | AYQSITVPIDASIPVLV | 95 |
| | ASIPVLVGPHMPGRTAAAR | | GPHMPGRTAAARACI | |
| | ACI* | | * | |
| IP 1-22 | RSDYKDDDDKAYQSITFD* | 82 | AYQSITFD* | 96 |
| IP 1-23 | RSDYKDDDDKFNRYQLMH | 83 | FNRYQLMHRYRY* | 97 |
| | RYRY* | | | |
| IP 2-01 | RSDYKDDDDKAYQSIRGVP | 84 | AYQSIRGVPMKVPID | 98 |
| | MKVPIDASIPVLVGPHMPGR | | ASIPVLVGPHMPGRT | |
| | TAAARACI* | | AAALRACI* | |
| IP 2-02 | RSDYKLDDDDKAYQSITRLIN | 85 | AYQSITRLIN* | 99 |
| | * | | | |
| IP 2-04 | DYKDDDDKAYQSISAYD* | 86 | AYQSISAYD* | 100 |
| IP 2-09 | RSDYKDDDDKAYQSIIR* | 87 | AYQSIIR* | 101 |
| IP 2-10 | RSDYKDDDDKAYLN* | 88 | AYLN* | 102 |

Comparison of the amino acid sequence of the identified peptides identified a number of consensus motifs that were present in a plurality of the identified peptides. For example, peptides IP 1-02, IP 1-04, IP 1-17, IP 1-18 and IP 2-01 each contained the following consensus motifs:

| | |
|---|---|
| PIDASIPVLVGPHMPGRT; | (SEQ ID NO: NO: 103) |
| and | |
| PIDASIPVLVGPHMPGRTAAARACI. | (SEQ ID NO: 104) |

Peptides IP 1-02, IP 1-04, IP 1-18 and IP 2-01 also contained the following consensus motifs:

| | |
|---|---|
| VPIDASIPVLVGPHMPGRT; | (SEQ ID NO: NO: 105) |
| and | |
| VPIDASIPVLVGPHMPGRTAAARACI. | (SEQ ID NO: 106) |

Peptides IP 1-01, IP 1-06 and IP 1-23 included the following consensus motif:

| | |
|---|---|
| YQLMQRYRY. | (SEQ ID NO: 107) |

Accordingly, these results indicate that peptides comprising these motifs are capable of reducing or inhibiting IRES-mediated translation.

### EXAMPLE 3

### Specific Inhibitors of HCV IRES-Mediated Translation

To identify peptides capable of specifically inhibiting or reducing IRES-mediated translation a vector was produced comprising the dsRED2 gene operably under the control of a promoter and the eGFP gene operably under the control of the HCV IRES. A peptide capable of reducing expression of the eGFP gene but not the dsRED2 gene is considered to reduce IRES-mediated translation but not Cap-dependent translation.

The vector was produced essentially as described in Example 1, however eGFP encoding nucleic acid was cloned downstream of the HCV IRES in place of the E. Coli gpt gene.

Monoclonal stably transfected cells were then produced essentially as described in Example 1.

The stably transfected cell line was then transiently transfected with a vector encoding a peptide isolated in the initial screen, as described in Example 2. Cells were transfected using Lipofectamine™ (Invitrogen Corporation) essentially according to manufacturer's instructions.

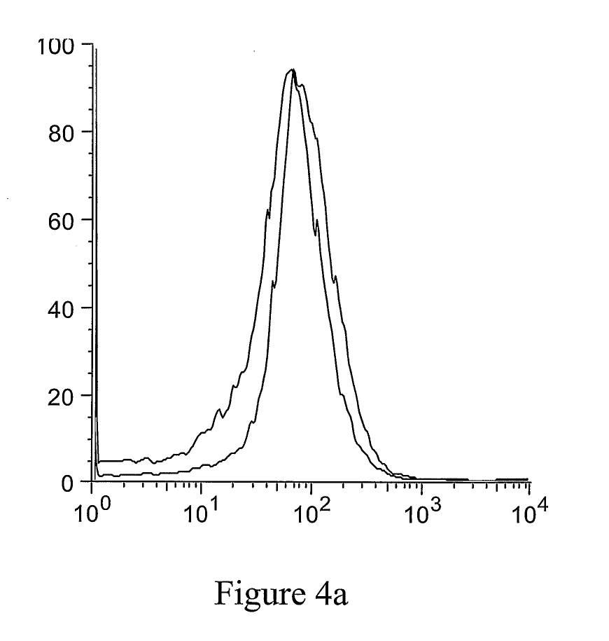

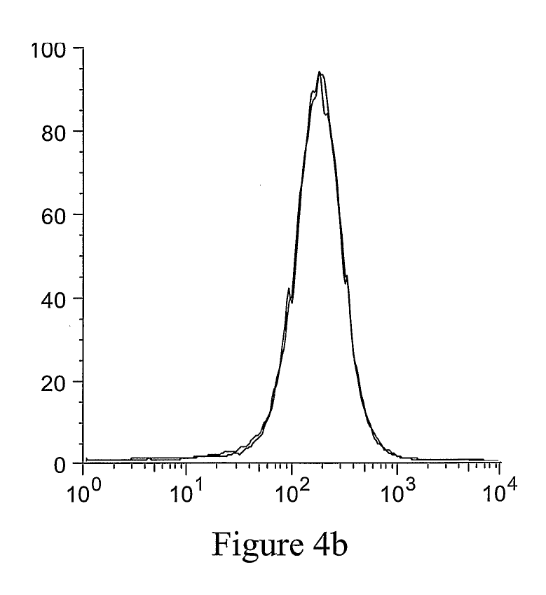

At 24 and 48 hours after transient transfection, the expression levels of both dsRED and eGFP were determined using FACS. Results of FACS analysis of cells are shown in FIG. 4. Those peptides that reduce eGFP expression levels but do not reduce dsRED expression levels were considered to express a peptide capable of specifically inhibiting or reducing IRES-mediated translation. A representative example of the FACS analysis is shown in FIG. 4.

Those peptides comprising the consensus motif PIDASIPVLVGPHMPGRT (SEQ ID NO: NO: 103) and/or PIDASIPVLVGPHMPGRTAAARACI (SEQ ID NO: 104) were shown to reduce eGFP expression in at least one assay.

### EXAMPLE 4

### Cells Expressing gpt Under Control of HCV IRES

### 4.1 Vector Construction

A vector comprising the fluorescent marker dsRED driven by the SV40 promoter and the E. coli gpt gene linked to the HCV IRES is produced to determine an inhibitor of HCV IRES-mediated translation.

Nucleic acid encoding dsRED is amplified using PCR using primers comprising the sequences ATGGCCTCCTCCGAGGAC (SEQ ID NO: 35) and GCCACCATCTGTTCCTTTAG (SEQ ID NO: 36). The amplified nucleic acid is then used to replace the puromycin resistance gene in pPUR-HCV (described by Welch et al., Gene Ther. 3: 994-1001, 1996) to produce pdsRED-HCV.

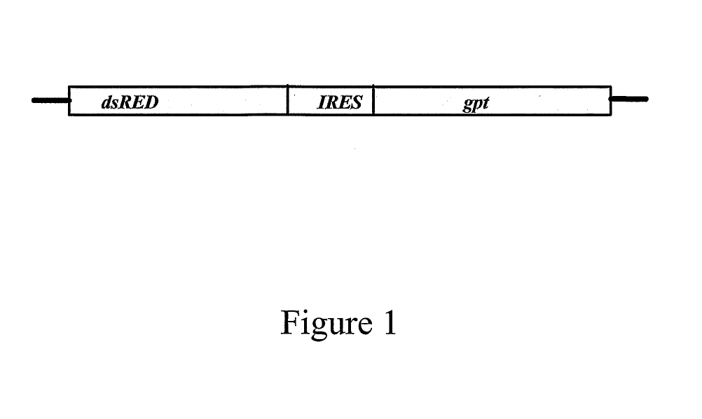

The E. coli gpt gene is then amplified using primers comprising the nucleotide sequence ATGAGCGAAAAATACATCGTC (SEQ ID NO: 37) or GCCAATCTCCGGTCGCTAA (SEQ ID NO: 38). The gpt gene is then inserted 3′ to the HCV 5′ region in the pdsRED-HCV vector to produce the pdsRED-HCV-gpt vector. The mRNA encoded by this vector is shown in FIG. 1.

The eGFP gene is also amplified using primers comprising the nucleotide sequence ATGGTGAGCAAGGGCGAG (SEQ ID NO: 39) or GGACGAGCTGTACAAGTAA (SEQ ID NO: 40). The amplified nucleic acid is then placed 3′ to the HCV 5′ region in the pdsRED-HCV vector (i.e., in place of the gpt gene) to produce the pdsRED-HCV-eGFP vector.

The pdsRED-HCV-eGFP is then further modified to insert a neomycin resistance gene linked to a CMV promoter into the vector to allow for selection.

### 4.2 Cell Transfection

The pdsRED-HCV-gpt vector is linearised and transfected into HEK-293 cells using Lipofectamine Plus (Invitrogen). 72 hours after transfection the medium on the cells is replaced with Eagle's medium with 10% dialysed fetal calf serum/xanthine (250 μg/ml)/hypoxanthine (15 μg/ml) or adenine (25 μg/ml)/L-glutamine (150 μg/ml)thymidine (10 μg/ml)/aminopterin (2 μg/ml)/mycophenolic acid (25 μg/ml). Twenty four hours later the medium is replaced with fresh medium containing the same supplements. Medium is then changed after 3 days.

Cells are maintained until colonies are formed and individual colonies isolated. Individual colonies are then grown and dsRED expression determined using fluorescence microscopy with a propidium iodide filter.

The pdsRED-HCV-eGFP is transfected into cells in a similar manner, however, G418 is added to standard culture medium to select for stable transformants. Furthermore, the expression of eGFP is detected.

### EXAMPLE 5

### Production of a Peptide Library for Screening for Anti-HCV IRES Activity

A peptide library is produced essentially as described in International application No. PCT/AU2004/000214. Nucleic acid is isolated from the following bacterial species: Archaeoglobus fulgidis, Aquifex aeliticus, Aeropyrum pernix, Bacillus subtilis, Bordetella pertussis TOX6, Borrelia burgdorferi, Chlamydia trachomatis, Escherichia coli K12, Haemophilus influenzae (rd), Helicobacter pylori, Methanobacterium thermoautotrophicum, Methanococcus jannaschii, Mycoplasma pneumoniae, Neisseria meningitidis, Pseudomonas aeruginosa, Pyrococcus horikoshii, Synechosistis PCC 6803, Thermoplasma volcanium and Thermotoga maritima.

Nucleic acid fragments are generated from the genomic DNA of each genome using 2 consecutive rounds of PCR using tagged random oligonucleotides. Samples are then purified and further amplified with primers that correspond to the tag region of the previously used primers and that comprise an EcoRI restriction endonuclease site.

Amplified products are then purified again and analyzed by electrophoresis on standard TAB-agarose gels to determine the approximate size of the nucleic acid fragments generated. The nucleic acid concentration of the samples is also determined.

PCR products from each of the 19 bacterial species are then pooled to generate a biodiverse nucleic acid library. To do so, DNA from each organism is added in an equimolar amount when compared to the amount of nucleic acid added to the pool from the organism with the smallest genome. Between 1 g and 10 μg of DNA from each organism is used, depending on the genome size of the organism from which the DNA is obtained.

In order to allow efficient cloning of the nucleic acid fragments into the pcDNA3.1 vector (Invitrogen), both the fragments and the vector are digested with the EcoRI restriction endonuclease.

Restriction digests are allowed to proceed overnight at 37° C. Samples are then purified using QIAquick PCR purification columns as per manufacturer's instructions. Nucleic acid is eluted into 50 μl of H20.

The fragments generated from the PCR products are then ligated into the digested pcDNA3.1 vector.

Ligation reactions are allowed to proceed overnight at 16° C. The ligase is then heat inactivated by incubating the samples at 65° C. for 30 minutes. Following completion of the ligation reaction samples are purified using an Amicon spin column. These columns are then centrifuged for 15 minutes at 3,800 rpm in a microcentrifuge.

Constructs is then cloned into the vector pcDNA3.1 and electrotransformed into DH10B.

Bacterial cells are grown in culture with amplicillin selection, and the vectors purified using standard techniques.

### EXAMPLE 6

### A Constrained Random Peptide Library

Two oligonucleotides encoding Cysteine or Proline constrained peptide libraries are synthesized. In both cases, 20 random codons are incorporated in a framework generated by constant cysteine or proline codons, flanked by sequences containing NcoI and BglII restriction sites for insertion in the vector pcDNA3.1 (Invitrogen). Oligonucleotide C5, encoding cysteine-constrained peptides, comprises the sequence GCGCATGCCATGGAGGGGATCCGATGT(NNK)₂TGT(NNK)₅TGT(NNK)₆TGT(N NK)₅TGT(NNK)₂TGTTAATAAGATCTCGCGTG (SEQ ID NO: 41). Oligonucleotide C8, encoding proline-constrained peptides, comprises a nucleotide sequence GCGCATGCCATGGAGGGGATCCGACCACCT(NNK)₅CCT(NNK)₅CCTCCACCT (NNK)₅CCT(NNK)₅CCACCTTAATAAGATCTCGCGTG (SEQ ID NO: 42).

Oligonucleotides are synthesized on an Applied Biosystems 392 synthesizer and contain triplets of the sequence NNK (where N is G, A, T, or C and K is G or C), which encodes all 20 amino acids but results in only one stop codon. To avoid synthesis bias, introduced by the chemical reactivity of each phosphoramidite, the N mix contains a final dN-CE phosphoramidite (Glen Research) concentration of 0.1 mmol/ml and a ratio of 3 dA/3 dC/2 dG/2 dT. In contrast, the K mix contains equal proportions of dG and dC.

Second-strand synthesis is performed by PCR with primers comprising a nucleotide sequence GCGCATGCCATGGAGGGGATCC (SEQ ID NO: 43) or CACGCGAGATCTTATTAA (SEQ ID NO: 44). Each of the constructs is then cloned into the vector pcDNA3.1 and electrotransformed into DH10B.

Bacterial cells are grown in culture with amplicillin selection, and the vectors purified using standard techniques.

### EXAMPLE 7

### Screening for Inhibitors of HCV IRES in Microplate Format

The expression vectors described in Examples 5 and 6 are separately transiently transfected into cells stably transfected with pdsRED-HCV-gpt. Two days following transfection cell culture medium is replaced with medium containing 100 μM 6-thioxanthine, and the cells maintained for three to five days.

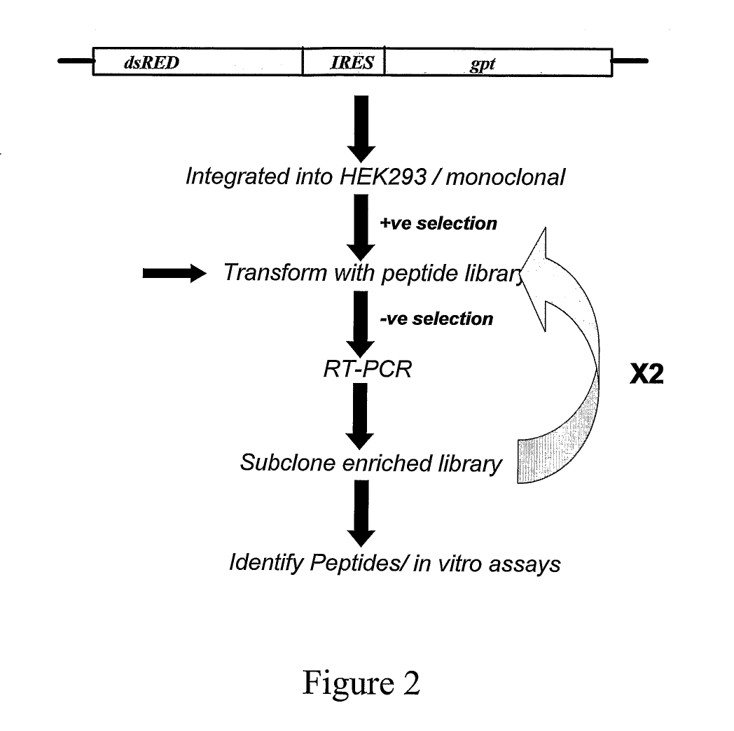

Those cells remaining are collected, lysed and nucleic acid isolated therefrom using standard methods. Each region encoding a peptide is isolated using PCR and cloned back into the pcDNA3.1 vector. These vectors are again screened to confirm that the encoded peptide is capable of suppressing HCV IRES-mediated translation of gpt. A schematic drawing of this screening process is shown in FIG. 2.

Following rescue of nucleic acid from the secondary screen and recloning, pdsRED-HCV-eGFP expressing cells are transfected with the inhibitory peptide encoding vectors. Control cells are transfected with empty pcDNA3.1 vector.

A constant number of cells (7.5×10⁵) expressing a specific peptide (or control) are then seeded into a clear-bottom 96-well microtitre plate and allowed to grow for 1 day. EGFP levels are measured in the intact cells (without removal of media), using a Fluostar microtiter plate fluorometer (Phoenix Research Products) with an excitation wavelength of 485 nm (25-nm bandwidth) and an emission wavelength of 515 nm (10-nm bandwidth). dsRED expression is determined using the fluorometer with an excitiation of 545 nm and an emission wavelength of 620 nm.

Results are normalised relative to cells transfected with the pcDNA3.1 vector with no insert. Samples are run in triplicate, and the fluorescent activity present in wells containing media only are subtracted from the fluorescent activity in all samples.

Those cells that express reduced levels of eGFP and an approximately equal level of dsRED when compared to a control cell are considered to express a peptide that selectively inhibits HCV IRES-mediated translation.

### EXAMPLE 8

### Selecting Cells Expressing Inhibitors of HCV IRES in Using Drug Selection and FACS Analysis

### 9.1 Drug Selection for Determining a HCV IRES Inhibitor

Expression vectors expressing inhibitors of HCV IRES-mediated translation are separately transiently transfected into cells stably transfected with pdsRED-HCV-gpt.

Two days following transfection, cell culture medium is replaced with medium containing 100 μM 6-thioxanthine, and the cells maintained for three to five days.

Cells capable of growth in the presence of thioxanthine are collected, lysed and nucleic acid isolated therefrom using standard methods. Each region encoding a putuative inhibitor peptide is isolated using PCR and re-cloned into the pcDNA3.1 vector.

The expression construct are again transfected into cells stably transfected with pdsRED-HCV-gpt to confirm that the encoded peptide is capable of suppressing HCV IRES-mediated translation of gpt.

Following rescue of nucleic acid from the secondary screen and recloning, pdsRED-HCV-eGFP expressing cells are transfected with the inhibitory peptide encoding vectors prior to FACS analysis of expression of dsRED and eGFP. Control cells are transfected with empty pcDNA3.1 vector.

### 8.2 FACS Analysis to Determine an IRES Specific Inhibitor

### 8.2.1 Cell Preparation

Transfected HEK293 adherent cells that have been transfected with a nucleic acid encoding a putative IRES inhibitor are trypsinised. The trypsin/EDTA solution is neutralised with 5 volumes of media (DMEM, 10% FCS, 2 mM L-glutamine, 50 U pen/strep). Cells are then centrifuged at 1,500 rpm for five minutes, supernatant discarded, and pellets resuspended in FACS buffer (PBS, 2% FCS, 0.02% sodium azide). A 400 μlaliquot of the cellular suspension is filtered through nylon membrane and 100 μl of propidium iodide (PI) (0.5 μg/ml in FACS buffer) added immediately prior to flow cytometry.

### 8.2.2 Flow Cytometry

Cells are analysed for dsRed and GFP fluorescence on a FACSCalibur machine (Becton Dickinson). Compensation is set using HEK293 cells transiently transfected with either pcDNA3-dsRed or pShuttle containing eGFP (positive controls) or untransfected HEK293 cells (negative control). The computer package Cellquest (V3.1) is used to set the parameters for the sample evaluation and to do a 4-colour analysis of the cells. Within the 4-colour analysis, channels FL1, FL2, and FL3 measure eGFP expression, dsRed expression, and PI, respectively, and FL4 (APC) is not recorded. Expression of dsRed and eGFP is evaluated with either Cellquest or FloJo (V.4) software.

Those samples that contain cells with high level of dsred expression and low levels of gfp expression are confirmed as containing inhibitors of IRES-mediated translation.

### EXAMPLE 9

### Selection of HCV IRES Inhibitors Using FACS

The expression vectors described in Examples 6 and 7 are separately transiently transfected into cells stably transfected with pdsRED-HCV-eGFP. Two days following transfection, the cells are prepared for high speed cell sorting using FACS. Those cells are then sorted and selected for cells expressing high levels of dsRED and low levels of eGFP as those containing a selective inhibitor of IRES-mediated translation initiation.

The population of cells that are determined to contain inhibitors of IRES-mediated translation are collected, lysed and nucleic acid isolated therefrom using standard methods. Each region encoding a peptide is isolated using PCR and cloned into the pcDNA3.1 vector. These vectors are then transformed into cells stably transfected with pdsRED-HCV-gpt confirm that the encoded peptide is capable of suppressing HCV IRES-mediated translation, i.e., by virtue of its ability to suppress expression of gpt (as described in Examples 2 and 8).

We claim:

1. A method for identifying a compound that reduces or inhibits internal ribosome entry site (IRES)-mediated translation, said method comprising:
(i) expressing in a cell a counter-selectable reporter gene operably linked to an IRES;
(ii) contacting the cell with or introducing into the cell a candidate compound under conditions sufficient to kill or inhibit or reduce the growth of a cell expressing the counter-selectable reporter gene; and
(iii) selecting a cell in which the expression of the counter-selectable reporter gene is reduced or inhibited wherein said reduced or inhibited expression is indicative of reduced or inhibited IRES-mediated translation,
thereby identifying a compound that reduces or inhibits IRES-mediated translation.

2. The method according to claim 1 wherein the IRES is from a virus.

3. The method according to claim 2 wherein the virus is hepatitis C virus.

4. The method according to claim 2 wherein the IRES comprises the nucleotide sequence set forth in SEQ ID NO: 6.

5. The method according to claim 1 wherein the counter selectable reporter gene is selected from the group consisting of herpes simplex virus thymidine kinase gene, cytosine deaminase gene, xanthine-guanine phosphoribosyltransferase (gpt) gene, hypoxanthine-guanine phosphoribosyltransferase gene, URA3, CYH2, LYS3 and a D-amino acid oxidase gene.

6. The method according to claim 1 wherein the counter selectable reporter gene is a xanthine-guanine phosphoribosyltransferase (gpt) gene and wherein conditions sufficient to kill or inhibit or reduce the growth of a cell expressing the counter-selectable reporter gene are achieved by contacting the cell with thioxanthine or 6-thioguanine.

7. The method according to claim 6 comprising a first step of maintaining the cell in the presence of xanthine as the sole precursor for guanine nucleotide formation and in the presence of one or more inhibitors that reduce or prevent de novo purine nucleotide synthesis to select a cell expressing the counter-selectable reporter gene.

8. The method according to claim 1 wherein the compound is a peptide.

9. The method according to claim 8 wherein the peptide is introduced into the cell by means of expressing nucleic acid encoding the peptide.

10. The method according to claim 9 further comprising introducing nucleic acid encoding the peptide into the cell.

11. The method according to claim 1 additionally comprising expressing in the cell a second reporter gene operably linked to a promoter the expression of which is not operably linked to the IRES at (i); and selecting a cell in which the expression of the counter-selectable reporter gene is reduced or inhibited and the expression of the second reporter gene is not detectably reduced or inhibited.

12. The method according to claim 11 wherein the second reporter gene encodes a fluorescent protein.

13. The method according to claim 12 wherein the fluorescent protein is a mutant discosoma red fluorescence protein.

14. The method according to claim 1 additionally comprising obtaining the compound from the cell or providing or producing the compound.

15. The method according to claim 1 additionally comprising providing, producing or obtaining the cell.

16. The method according to claim 14 additionally comprising:
(i) expressing in a cell a first reporter gene other than a counter-selectable reporter gene operably linked to a promoter and a second reporter gene operably linked to the IRES;
(ii) contacting the cell with or introducing into the cell the identified compound under conditions sufficient for expression of the first and second reporter genes; and
(iii) selecting a cell in which the expression of the first reporter gene is not detectably reduced and the expression of the second reporter gene is reduced or inhibited and said reduced or inhibited expression indicates that the compound that selectively reduces or inhibits IRES-mediated translation.

17. The method according to claim 16 wherein the first and second reporter genes each encode a fluorescent protein.

18. The method according to claim 17 wherein the first reporter gene encodes a green fluorescence protein and the second reporter gene encodes a mutant discosoma red fluorescence protein.

19. The method of claim 1 comprising:
(i) expressing in a cell a xanthine-guanine phosphoribosyltransferase (gpt) gene counter-selectable reporter gene comprising the nucleotide sequence set forth in SEQ ID NO: 13 operably linked to an IRES;
(ii) expressing in the cell a candidate peptide;
(iii) maintaining the cell in the presence of thioxanthine or 6-thioguanine for a time and under conditions sufficient to kill or inhibit or reduce the growth of a cell expressing the counter-selectable reporter gene; and
(iv) selecting a cell in which the expression of the counter-selectable reporter gene is reduced or inhibited and said reduced or inhibited expression is indicative of reduced or inhibited IRES-mediated translation,
thereby identifying a compound that reduces or inhibits IRES-mediated translation.

20. The method of claim 1 identifying a peptide that selectively inhibits or reduces internal ribosome entry site (IRES)-mediated translation, said method comprising:
(i) expressing in a cell a xanthine-guanine phosphoribosyltransferase (gpt) gene counter-selectable reporter gene comprising the nucleotide sequence set forth in SEQ ID NO: 13 operably linked to an IRES;
(ii) expressing in the cell a candidate peptide;
(iii) maintaining the cell in the presence of thioxanthine or 6-thioguanine for a time and under conditions sufficient to kill or inhibit or reduce the growth of a cell expressing the counter-selectable reporter gene;
(iv) selecting a cell in which the expression of the counter-selectable reporter gene is reduced or inhibited and said reduced or inhibited expression is indicative of reduced or inhibited IRES-mediated translation and identifying the peptide expressed by the selected cell;
(v) expressing the identified peptide in a cell additionally expressing (a) a first reporter gene encoding a green fluorescence protein comprising the amino acid sequence set forth in SEQ ID NO: 20 said first reporter gene operably linked to a promoter; and (b) a second reporter gene encoding a mutant discosoma red fluorescence protein comprising an amino acid sequence set forth in SEQ ID NO: 26 said second reporter gene operably linked to the IRES; and
(vi) selecting a cell in which the expression of the first reporter gene is not detectably reduced and the expression of the second reporter gene is reduced or inhibited and said reduced or inhibited expression is indicative of reduced IRES-mediated translation,
thereby identifying a peptide that selectively reduces or inhibits IRES-mediated translation.

21. A process for providing a compound that inhibits or reduces IRES-mediated translation, said process comprising:
(i) performing the method according to claim 1 to identify a compound that inhibits or reduces IRES-mediated translation;
(ii) optionally, isolating the compound or a nucleic acid encoding the compound;
(iii) optionally, determining the structure of the compound; and
(iv) providing the compound or the name or structure of the compound.

22. An isolated or recombinant peptide or peptide analogue selected from the group consisting of:
(i) a peptide consisting of an amino acid sequence selected from the group consisting of SEQ ID NO: 103, SEQ ID NO: 105, SEQ ID NO: 107, SEQ ID NO: 108, SEQ ID NO: 109 and SEQ ID NO: 110, optionally comprising an N-terminal methionine residue;
(ii) a peptide or peptide analogue encoded by a nucleic acid consisting of a sequence selected from the group consisting of SEQ ID NO: 125, SEQ ID NO: 126, SEQ ID NO: 127, SEQ ID NO: 128, SEQ ID NO: 129, SEQ ID NO: 130, SEQ ID NO: 131, SEQ ID NO: 132, SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147 and SEQ ID NO: 148, optionally comprising a 5′ sequence encoding a methionine residue;
(iii) a peptide comprising an amino acid sequence selected from the group consisting of SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO: 88, SEQ ID NO: 89, SEQ ID NO: 90, SEQ ID NO: 91, SEQ ID NO: 92, SEQ ID NO: 93, SEQ ID NO: 94, SEQ ID NO: 95, SEQ ID NO: 96, SEQ ID NO: 97, SEQ ID NO: 98, SEQ ID NO: 99, SEQ ID NO: 100, SEQ ID NO: 101, SEQ ID NO: 102, SEQ ID NO: 104 and SEQ ID NO: 106;
(iv) a peptide or peptide analogue encoded by a nucleic acid comprising a sequence selected from the group consisting of SEQ ID NO: 61, SEQ ID NO: 62, SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73 and SEQ ID NO: 74 SEQ ID NO: 111, SEQ ID NO: 112, SEQ ID NO: 113, SEQ ID NO: 114, SEQ ID NO: 115, SEQ ID NO: 116, SEQ ID NO: 117, SEQ ID NO: 118, SEQ ID NO: 119, SEQ ID NO: 120, SEQ ID NO: 121, SEQ ID NO: 122, SEQ ID NO: 123 and SEQ ID NO: 124; and
(v) an analogue of any one of (i) to (iv) selected from the group consisting of (a) the sequence of any one of (i) to (iv) comprising one or more non-naturally-occurring amino acids; (b) the sequence any one of (i) to (iv) comprising one or more non-naturally-occurring amino acid analogues; (c) an isostere of any one of (i) to (iv); (d) a retro-peptide analogue of any one of (i) to (iv); and (e) a retro-inverted peptide analogue of any one of (i) to (iv).

23. The isolated or recombinant peptide or peptide analogue according to claim 66 selected from the group consisting of:
(i) a peptide comprising an amino acid sequence set forth in SEQ ID NO: 104;
(ii) an analogue of (i) selected from the group consisting of (a) the sequence of (i) comprising one or more non-naturally-occurring amino acids; (b) the sequence of (i) comprising one or more non-naturally-occurring amino acid analogues; (c) an isostere of (i); (d) a retro-peptide analogue of (i); and (e) a retro-inverted peptide analogue of (i).

24. The isolated or recombinant peptide or peptide analogue according to claim 22 wherein the peptide at (iii) or (iv) comprises a protein transduction domain.

25. The isolated or recombinant peptide or peptide analogue according to claim 24 wherein the protein transduction domain is a HIV-1 TAT protein transduction domain.

26. The isolated or recombinant peptide or peptide analogue according to claim 22 wherein the analogue comprises one or more D amino acids.

27. The isolated or recombinant peptide or peptide analogue according to claim 22 wherein the analogue is a retro-inverted peptide analogue.

28. A pharmaceutical composition comprising the isolated or recombinant peptide or peptide analogue according to claim 22 and a pharmaceutically acceptable carrier or excipient.

29. A pharmaceutical composition comprising a nucleic acid that encodes the isolated or recombinant peptide or peptide analogue according to any claim 22 and a pharmaceutically acceptable carrier or excipient.

30. The pharmaceutical composition according to claim 28 additionally comprising an antiviral agent.

31. A method for providing or producing an isolated or recombinant peptide or peptide analogue according to claim 22 comprising providing or obtaining a sequence of the peptide or peptide analogue or a sequence of nucleic acid encoding the peptide or peptide analogue and synthesizing or expressing the peptide or peptide analogue.

32. A method of therapeutic or prophylactic treatment of a subject comprising administering an isolated or recombinant peptide or peptide analogue according to claim 22 to a subject in need thereof.

33. The method according to claim 32 wherein the therapeutic or prophylactic treatment comprises treating or preventing a viral infection in a subject.

34. Use of the isolated or recombinant peptide or peptide analogue according to claim 22 in medicine.

35. A method of treating or preventing a viral infection, said method comprising administering one or more peptides or peptide analogues or a pharmaceutical composition comprising said one or more peptides or peptide analogues to a subject in need thereof, wherein a peptide or peptide analogue is selected from the group consisting of:
(i) a peptide comprising an amino acid sequence selected from the group consisting of SEQ ID NO: 103, SEQ ID NO: 105, SEQ ID NO: 107, SEQ ID NO: 108, SEQ ID NO: 109 and SEQ ID NO: 110;
(ii) an analogue of (i) selected from the group consisting of (a) the sequence of (i) comprising one or more non-naturally-occurring amino acids; (b) the sequence of (i) comprising one or more non-naturally-occurring amino acid analogues; (c) an isostere of (i); (d) a retro-peptide analogue of (i); and (e) a retro-inverted peptide analogue of (i).

36. The method according to claim 33 wherein the viral infection is a hepatitis C viral infection.

37. The method according to claim 35 wherein the viral infection is a hepatitis C viral infection.

38. Use of one or more peptides or peptide analogues in the manufacture of a medicament for the treatment of a viral infection, wherein the peptide or peptide analogue is selected from the group consisting of:
(i) a peptide comprising an amino acid sequence selected from the group consisting of SEQ ID NO: 103, SEQ ID NO: 105, SEQ ID NO: 107, SEQ ID NO: 108, SEQ ID NO: 109 and SEQ ID NO: 110;
(ii) an analogue of (i) selected from the group consisting of (a) the sequence of (i) comprising one or more non-naturally-occurring amino acids; (b) the sequence of (i) comprising one or more non-naturally-occurring amino acid analogues; (c) an isostere of (i); (d) a retro-peptide analogue of (i); and (e) a retro-inverted peptide analogue of (i); and
(iii) the peptide or peptide analogue according to claim 22.


SEQUENCE LISTING (148 sequences)

SEQ ID NO: 1 - DNA, 743 bp, poliovirus
ttaaaacagctctggggttgtacccaccccagaggcccacgtggcggctagtactccggtattgcggtacccttgtacgcctgttttatactcccttcccgtaacttagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttggttagcactcaaccccagagtgtagcttaggctgatgagtctggacatccctcaccggtgacggtgttccaggctgcgttggcggcctacctatggctaacgcatgggacgctagttgtgaacaaggtgtgaagagcctattgagctacataagaatcctccggcccctgaatgcggctaatcccaacctcggagcaggtggttcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatg

SEQ ID NO: 2 - DNA, 621 bp, rhinovirus
ttaaaactgggagtgggttgttcccactcactccacccatgcggtgttgtactctgttattacggtaactttgtacgccagtttttcccacccttccccataatgtaacttagaagtttgtacaatatgaccaataggtgacaatcatccagactgtcaaaggtcaagcacttctgtttccccggtcaatgaggatatgctttacccaaggcaaaaaccttagagatcgttatccccacactgcctacacagagcccagtaccatttttgatataattgggttggtcgctccctgcaaacccagcagtagacctggcagatgaggctggacattccccactggcgacagtggtccagcctgcgtggctgcctgctcacccttcttgggtgagaagcctaattattgacaaggtgtgaagagccgcgtgtgctcagtgtgcttcctccggcccctgaatgtggctaaccttaaccctgcagccgttgcccataatccaatgggtttgcggtcgtaatgcgtaagtgcgggatgggaccaactactttgggtgtccgtgtttcctgtttttcttttgattgcattttatggtgacaatttatagtgtatagattgtcatcatg

SEQ ID NO: 3 - DNA, 621 bp, hepatitis A; identical to SEQ ID NO: 2

SEQ ID NO: 4 - DNA, 234 bp, human immunodeficiency virus-1
tgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatg

SEQ ID NO: 5 - DNA, 614 bp, hepatitis C virus
agaccacaacggtttccctctagcgggatcaattccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgataataccatg

SEQ ID NO: 6 - DNA, 363 bp, hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggaaggccatagtggtctgcggaaccggtgagtacaccggaattgccaggacgaccgggtcctttcttggattaaccccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcatcatgagcacaaatcctaaacct

SEQ ID NO: 7 - DNA, 1041 bp, foot and mouth disease virus
ttgaaagggggcgctagggtctcacccctaacatgccaacgacagtccctgcattgcactccacacttacgttgtgcgtacgcggggcccgatggaccatcgttcacccacctacagctggactcacgacaccgcgtggccattttagctggattgtgcggacgaacaccgcttgcgcttctcgcgtgaccggttagtactcttaccaccttccgcctacttggttgttagcgctgtcctgggcacccctgttgggggccgttcgacgctccacgggttcccccgtgcggcaactacggtgatggggccgtttcgcgcgggctgatcgcctggtttgcttcggctgtcacccgaaacccgcctttcataagttttaccgtctgtcccgacgttaaagggaggtaaccacaagcatactgccgccttccccggcgttaacgggatgcaaccgtaagacacaccttcatccggaagtgaaacggctgcgtcacatagttttgcccgttttcacgagaaatgggacgtctgcgcacgaaacgcgccgtcgcttgaggaagacttgtacaaacacgatctaagcaggtttccccaactgacacaaaacgtgcaacttgaaactccgcctggtctttccaggtctagaggggtaacactttgtactgtgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttacaattaatgaccctatg

SEQ ID NO: 8 - DNA, 410 bp, Homo sapiens
caggatccccctaattccagcgagaggcagagggagcgagcgggcggccggctagggtggaagagccgggcgagcagagctgcgctgcgggcgtcctgggaagggagatccggagcgaatagggggcttcgcctctggcccagccctcccgctgatcccccagccagcggtccgcaacccttgccgcatccacgaaactttgcccatagcagcgggcgggcactttgcactggaacttacaacacccgagcaaggacgcgactctcccgacgcggggaggctattctgcccatttggggacacttccccgccgctgccaggacccgcttctctgaaaggctctccttgcagctgcttagacgctggatttttttcgggtagtggaaaaccagcagcctcccgcgaccatg

SEQ ID NO: 9 - DNA, 201 bp, Homo sapiens
tgaatgatgtggtaatgtcgaactctagtatttagaattagaatgtttcttagcggtcgtgtagttatttttatgtcataagtggataatttgttagctcctataacaaaagtctgttgcttgtgtttcacattttggatttcctaatataatgttctctttttagaaaaggtggacaagtcctattttcaagagaagatg

SEQ ID NO: 10 - DNA, 580 bp, Homo sapiens
aagaagaggtagcgagtggacgtgactgctctatcccgggcaaaagggatagaaccagaggtggggagtctgggcagtcggcgacccgcgaagacttgaggtgccgcagcggcatccggagtagcgccgggctccctccggggtgcagccgccgtcgggggaagggcgccacaggccgggaagacctcctccctttgtgtccagtagtggggtccaccggagggcggcccgtgggccgggcctcaccgcggcgctccgggactgtggggtcaggctgcgttgggtggacgcccacctcgccaaccttcggaggtccctgggggtcttcgtgcgccccggggctgcagagatccaggggaggcgcctgtgaggcccggacctgccccggggcgaagggtatgtggcgagacagagccctgcacccctaattcccggtggaaaactcctgttgccgtttccctccaccggcctggagtctcccagtcttgtcccggcagtgccgccctccccactaagacctaggcgcaaaggcttggctcatggttgacagctcagagagagaaagatctgagggaagatg

SEQ ID NO: 11 - DNA, 497 bp, Homo sapiens
actagttctagaccgccgaactcaggccggccccagaaaacccgagcgagtagggggcggcgcgcaggagggaggagaactgggggcgcgggaggctggtgggtgtcgggggtggagatgtagaagatgtgacgccgcggcccggcgggtgccagattagcggacgcggtgcccgcggttgcaacgggatcccgggcgctgcagcttgggaggcggctctccccaggcggcgtccgcggagacacccatccgtgaaccccaggtcccgggccgccggctcgccgcgcaccaggggccggcggacagaagagcggccgagcggctcgaggctgggggaccgcgggcgcggccgcgcgctgccgggcgggaggctggggggccggggccggggccgtgccccggagcgggtcggaggccggggccggggccgggggacggcggctccccgcgcggctccagcggctcggggatcccggccgggccccgcagggaccatg

SEQ ID NO: 12 - DNA, 297 bp, Homo sapiens
gctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatg

SEQ ID NO: 13 - DNA, 459 bp, Escherichia coli
  CDS (1)..(459)
atgagcgaaaaatacatcgtcacctgggacatgttgcagatccatgcacgtaaactcgcaagccgactgatgccttctgaacaatggaaaggcattattgccgtaagccgtggcggtctggtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaa

SEQ ID NO: 14 - protein, 152 aa, Escherichia coli
MSEKYIVTWDMLQIHARKLASRLMPSEQWKGIIAVSRGGLVPGALLARELGIRHVDTVCISSYDHDNQRELKVLKRAEGDGEGFIVIDDLVDTGGTAVAIREMYPKAHFVTIFAKPAGRPLVDDYVVDIPQDTWIEQPWDMGVVFVPPISGR

SEQ ID NO: 15 - DNA, 1545 bp, Mus musculus
  CDS (48)..(704)
ccattgtgctggaaagcagcagcctgcccggcgggccgcgccgctccatggcgactcacagcccctgcatcgtgattggcgatgatgaacaaggttacgacctggacttgttctgcatacccaaacattatgcagacgatctggagaaagtctatatccctcatgggctcatcatggacaggacagagagactggcacgtgaaattatgaagggcatgggaggacaccacattgtagctctctgtgtactcaagggtggctataaattttttgctgatctgttagactacatcaaagcactgaacagaaacagtgacaagtcaatccccatgactgtggacttcattaggttgaagagttactgtaatgatcagtcaactggagatataaaagtcattggtggggatgacctctcaaccttgactggaaagaatgttttaattgtagaagatataattgatactgggaaaacaatgaaaacgttgctgtctctacttaagcagtacaatccaaagatggtgaaagtggccagtttgttggtcaaaagaactcctcgaagtgtgggatatcggccagactttgttggatttgaagtgccagacaaatttgttgttggatacgccctcgactacaatgaatacttcagagatttgaatcatatctgtgtgatcagtgagacggggaagcagaagtacaaagcatgaaagcacagttgaagtgctatggctacaaagctttgaaatgttttgtttaccaagtcctatctctcacaggcttcaactctggtacaccagctgaaattgtagaacaccccagccccatcgtcatatgcttactataacttattgcatgtacaatataagaatctcgtcttggtcatttatattatagaaatgtaaccacccagtgcaattctgcactccttattttgatttgcactatgactctaccgactattgctgcccctggttgggttgtgctgtttgtgagctccggagtctaactcttgcagtggtaaattgcttaaacctcatcaacccagaactgaaatagttcgagtactgtaaatgtaaaacattttatgatagggaagtctattagtaatattttttaaaaactgtaatttaagttttatattttaattcacaaatgtgattgtgatgaaaggatagttgcacctttgggtgttatgaaacacaagaagcagccagttacagtatctgtaatctccagggagcccactgacacctcctggagttgccttattgctgtaaaaaggaatctgggtaaaaaatatctttttttcttcttcttctttttgagatggaatgacaaaacagaatgcttgaggtctagttttagttgagctgcctgtttctttattttttttttctttaaaaatatccatcagacagtgaaattgccttttaaatttaaacagttttaatatatttgtgggaaaaaaaaaatatgtaatgttaactttataagaactacaaagcaaagtactttaaataaaggctgtctctttaaaataaaaaaaaa

SEQ ID NO: 16 - protein, 218 aa, Mus musculus
MATHSPCIVIGDDEQGYDLDLFCIPKHYADDLEKVYIPHGLIMDRTERLAREIMKGMGGHHIVALCVLKGGYKFFADLLDYIKALNRNSDKSIPMTVDFIRLKSYCNDQSTGDIKVIGGDDLSTLTGKNVLIVEDIIDTGKTMKTLLSLLKQYNPKMVKVASLLVKRTPRSVGYRPDFVGFEVPDKFVVGYALDYNEYFRDLNHICVISETGKQKYKA

SEQ ID NO: 17 - DNA, 1633 bp, Homo sapiens
  CDS (201)..(1244)
ttggggtccattgcaacccgaggcgagactagagttcccaagcgagaagggaagaggcagtgggtgcacgtggaaggcggacagagggctggaaacaagacgctccagaatcaggagcttcccctcaggaaatagcatcctgtgtccccgcactgcagttgtctggtctctccagcagtttggtacttccggctgctgcaatgcgtgtggtggtgattggagcaggagtcatcgggctgtccaccgccctctgcatccatgagcgctaccactcagtcctgcagccactgcacataaaggtctacgcggaccgcttcaccccactcaccaccaccgacgtggctgccggcctctggcagccctacctttctgaccccaacaacccacaggaggcggactggagccaacagacctttgactatctcctgagccatgtccattctcccaacgctgaaaacctgggcctgttcctaatctcgggctacaacctcttccatgaagccattccggacccttcctggaaggacacagttctgggatttcggaagctgacccccagagagctggatatgttcccagattacggctatggctggttccacacaagcctaattctggagggaaagaactatctacagtggctgactgaaaggttaactgagaggggagtgaagttcttccagcggaaagtggagtcttttgaggaggtggcaagagaaggcgcagacgtgattgtcaactgcactggggtatgggctggggcgctacaacgagaccccctgctgcagccaggccgggggcagatcatgaaggtggacgccccttggatgaagcacttcattctcacccatgacccagagagaggcatctacaattccccgtacatcatcccagggacccagacagttactcttggaggcatcttccagttgggaaactggagtgaactaaacaatatccaggaccacaacaccatttgggaaggctgctgcagactggagcccacactgaagaatgcaagaattattggtgaagcaactggcttccggccagtacgcccccagattcggctagaaagagaacagcttcgcactggaccttcaaacacagaggtcatccacaactatggccatggaggctacgggctcaccatccactggggatgtgccctggaggcagccaagctctttgggagaatcctggaagaaaagaaattgtccagaatgccaccatcccacctctgaagactccagtgactgctgcctccccccacaagaactcccttctcccctcagccaatgaatcaatgtgctccttcataagccattgcttctccctcacttctttcctcaaagaagcatgaggtgagagaaagccacraagtcagtgcctggagaagggttcagcccaacatggggcccctctcatcactgaaatccctctaccttctctgggtctggcattataaagaacagctgaggctgtcattccatgagtcttcagaagaaaggacagctcagaaagtcaaagaggccaactgcccagagccacagaaaatggaggataattgaggctaagtaacctgattacaagttgtactaacatattaaaggttctgaaaagtcctgcaaaa

SEQ ID NO: 18 - protein, 347 aa, Homo sapiens [sequence omitted]

SEQ ID NO: 19 - DNA, 720 bp, Artificial sequence
  enhanced green fluorescence protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 20 - protein, 239 aa, Artificial sequence
  Synthetic Construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 21 - DNA, 696 bp, Artificial sequence
  cyan fluorescence protein
atgtcttattcaaagcaaggcatcgcacaagtaatgaagacgaaataccatatggaaggcagtgtcaatggccatgaattcacgatcgaaggtgtaggaactggaaacccttacgaaggcacacagatgtccgaattagtgatcaccaagcctgcaggaaaaccccttccattctcctttgacattctgtcaacagtctttcaatatggaaacaggtgcttcacaaagtaccctgaaggaatgactgactatttcaagcaagcattcccagatggaatgtcatatgaaaggtcatttctatatgaggatggaggagttgctacagccggctggaacattggtcttgagagagattgcttcatccacaaatccatctatcatggcgttaactttcccgctgatggacccgtaatgaaaaagaagaccattggctgggataaagccttcgaaaaaatgactgtgtccaaagacgtgttaagaggtgatgtgactgggtttcttatgctcgaaggaggtggttaccacagctgccagtttcactccacttacaaaccagaaaagccggctgcactgcccccgaatcatgtcgtagaacatcacattgtgaggactgaccttggccaaagtgcaaaaggcttcacagtcaagctggaagaacatgctgcggctcatgttaaccctttgaaggttcaataa

SEQ ID NO: 22 - protein, 231 aa, Artificial sequence
  Synthetic Construct
MSYSKQGIAQVMKTKYHMEGSVNGHEFTIEGVGTGNPYEGTQMSELVITKPAGKPLPFSFDILSTVFQYGNRCFTKYPEGMTDYFKQAFPDGMSYERSFLYEDGGVATAGWNIGLERDCFIHKSIYHGVNFPADGPVMKKKTIGWDKAFEKMTVSKDVLRGDVTGFLMLEGGGYHSCQFHSTYKPEKPAALPPNHVVEHHIVRTDLGQSAKGFTVKLEEHAAAHVNPLKVQ

SEQ ID NO: 23 - DNA, 678 bp, Artificial sequence
  Yellow fluorescence protein
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttcatgtggggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggcggccaccaccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagattgacgggaagctggacatcacctcccacaacgaggactacaccatcgtggaacagtacgagcgcgccgagggccgccactccaccggcgcctaa

SEQ ID NO: 24 - protein, 225 aa, Artificial sequence
  Synthetic Construct
MASSEDVIKEFMRFKVRMEGSVNGHEFEIEGEGEGRPYEGTQTAKLKVTKGGPLPFAWDILSPQFMWGSKAYVKHPADIPDYLKLSFPEGFKWERVMNFEDGGVVTVTQDSSLQDGEFIYKVKLRGTNFPSDGPVMQKKTMGWAATTERMYPEDGALKGEIKMRLKLKDGGHYDAEVKTTYMAKKPVQLPGAYKIDGKLDITSHNEDYTIVEQYERAEGRHSTGA

SEQ ID NO: 25 - DNA, 678 bp, Artificial sequence
  mutant discosoma red fluorescence protein
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggtgaccgtgacccaggactcctccctgcaggacggctgcttcatctacaaggtgaagttcatcggcgtgaacttcccctccgacggccccgtaatgcagaagaagactatgggctgggagccctccaccgagcgcctgtacccccgcgacggcgtgctgaagggcgagatccacaaggccctgaagctgaaggacggcggccactacctggtggagttcaagtccatctacatggccaagaagcccgtgcagctgcccggctactactacgtggactccaagctggacatcacctcccacaacgaggactacaccatcgtggagcagtacgagcgcaccgagggacgccaccatctgttcctttag

SEQ ID NO: 26 - protein, 225 aa, Artificial sequence
  Synthetic Construct
MASSEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGCFIYKVKFIGVNFPSDGPVMQKKTMGWEPSTERLYPRDGVLKGEIHKALKLKDGGHYLVEFKSIYMAKKPVQLPGYYYVDSKLDITSHNEDYTIVEQYERTEGRHHLFL

SEQ ID NO: 27 - DNA, 687 bp, artificial sequence
  mutant discosoma red fluorescence protein 2
gggatcctgatggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagaccatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctgtga

SEQ ID NO: 28 - protein, 228 aa, artificial sequence
  Synthetic Construct
GILMASSENVITEFMRFKVRMEGTVNGHEFEIEGEGEGRPYEGHNTVKLKVTKGGPLPFAWDILSPQFQYGSKVYVKHPADIPDYKKLSFPEGFKWERVMNFEDGGVATVTQDSSLQDGCFIYKVKFIGVNFPSDGPVMQKKTMGWEASTERLYPRDGVLKGETHKALKLKDGGHYLVEFKSIYMAKKPVQLPGYYYVDAKLDITSHNEDYTIVEQYERTEGRHHLFL

SEQ ID NO: 29 - DNA, 678 bp, Artificial sequence
  monomeric orange fluorescence protein
atggcctcctccgaggacgtcatcaaggagttcatgcgcttcaaggtgcgcatggagggctccgtgaacggccacgagttcgagatcgagggcgagggcgagggccgcccctacgagggcacccagaccgccaagctgaaggtgaccaagggcggccccctgcccttcgcctgggacatcctgtcccctcagttctgttacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagatgaggctgaagctgaaggacggcggccactacgacgccgaggtcaagaccacctacatggccaagaagcccgtgcagctgcccggcgcctacaagaccgacatcaagctggacatcacctcccacaacgaggactacaccatcgtggaattgtacgagcgcgccgagggccgccactccaccggcgcctaa

SEQ ID NO: 30 - protein, 225 aa, Artificial sequence
  Synthetic Construct
MASSEDVIKEFMRFKVRMEGSVNGHEFEIEGEGEGRPYEGTQTAKLKVTKGGPLPFAWDILSPQFCYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKMRLKLKDGGHYDAEVKTTYMAKKPVQLPGAYKTDIKLDITSHNEDYTIVELYERAEGRHSTGA

SEQ ID NO: 31 - DNA, 941 bp, Aquorea coerulescens
  CDS (67)..(783)
attcaaaacactgcagaattttggatagattttcctgctacttcacacgcataaaagacaagaaagatgagtaaaggagcagaacttttcactggagttgtcccaattcttattgaattaaatggtgatgttaatgggcacaaattctctgtcagtggagagggcgaaggtgatgcgacatacggaaagttaacccttaaatttatttgcactacaggaaaactacctgttccatggccaacacttgtcactactttctcttatggtgttcaatgcttttcaagatatccagatcatatgaaacagcatgacttcttcaagagtgccatgcctgaaggttatatacaggaaagaactatatttttcaaagatgacgggaactacaagtcgcgtgctgaagtcaagttcgaaggtgataccctggttaatagaattgagttaacaggtactgattttaaagaagatggaaacatccttggaaataaaatggaatacaactataacgcacataatgtatacatcatgacagacaaagcaaaaaatggaatcaaagttaacttcaaaattagacacaacattgaagatggaagcgttcaacttgcagaccattatcaacaaaatactccaattggcgatggccctgtccttttaccagataaccattacctgtccacacaatctaccctttccaaagatcccaacgaaaagagagatcacatgatctattttgagtttgtaacagctgctgcgattacacatggcatggatgaattatacaaataaatgtatagacttcaagttgacactaacgtgtccgaacaattactaaaatctcagggttcctggttaaaatcaggctgagatattatttacatattatagattcattagaattatttaaatactttatagatgttattgataggggttattttcttatt

SEQ ID NO: 32 - protein, 238 aa, Aquorea coerulescens
MSKGAELFTGVVPILIELNGDVNGHKFSVSGEGEGDATYGKLTLKFICTTGKLPVPWPTLVTTFSYGVQCFSRYPDHMKQHDFFKSAMPEGYIQERTIFFKDDGNYKSRAEVKFEGDTLVNRIELTGTDFKEDGNILGNKMEYNYNAHNVYIMTDKAKNGIKVNFKIRHNIEDGSVQLADHYQQNTPIGDGPVLLPDNHYLSTQSTLSKDPNEKRDHMIYFEFVTAAAITHGMDELYK

SEQ ID NO: 33 - protein, 11 aa, Artificial sequence
  HIV-1 TAT protein transduction domain
GRKKRRQRRRG

SEQ ID NO: 34 - protein, 27 aa, Artificial sequence
  signal sequecne-based peptide protein transduction domain
GALFLGWLGAAGSTMGAWSQPKKKRKV

SEQ ID NO: 35 - protein, 16 aa, Artificial sequence
  signal based sequence-based peptide protein transduction domain
AAVALLPAVLLALLAP

SEQ ID NO: 36 - protein, 26 aa, Artificial sequence
  Transportan protein transduction domain
GWTLNSAGYLLKINLKALAALAKKIL

SEQ ID NO: 37 - protein, 18 aa, Artificial sequence
  Amphiphilic model peptide protein transduction domain
KLALKLALKALKAALKLA

SEQ ID NO: 38 - protein, 11 aa, Artificial sequence
  Polyarginine protein transduction domain
RRRRRRRRRRR

SEQ ID NO: 39 - DNA, 18 bp, Artificial sequence
  oligonucleotide for amplifying dsRed
atggcctcctccgaggac

SEQ ID NO: 40 - DNA, 20 bp, Artificial sequence
  oligonucleotide for amplifying dsRed
gccaccatctgttcctttag

SEQ ID NO: 41 - DNA, 21 bp, Artificial sequence
  oligonucleotide for amplifying gtp
atgagcgaaaaatacatcgtc

SEQ ID NO: 42 - DNA, 19 bp, Artificial sequence
  oligonucleotide for amplifying gtp
gccaatctccggtcgctaa

SEQ ID NO: 43 - DNA, 18 bp, Artificial sequence
  oligonucleotide for amplifying gtp
atggtgagcaagggcgag

SEQ ID NO: 44 - DNA, 19 bp, Artificial sequence
  oligonucleotide for amplifying gtp
ggacgagctgtacaagtaa

SEQ ID NO: 45 - DNA, 119 bp, Artificial sequence
  oligonucleotide for producing random peptide library
gcgcatgccatggaggggatccgatgtnnknnktgtnnknnknnknnknnktgtnnknnknnknnknnknnktgtnnknnknnknnknnktgtnnknnktgttaataagatctcgcgtg

SEQ ID NO: 46 - DNA, 125 bp, Artificial sequence
  oligonucleotide for producing random peptide library
gcgcatgccatggaggggatccgaccacctnnknnknnknnknnkcctnnknnknnknnkcctccacctnnknnknnknnknnkcctnnknnknnknnknnkccaccttaataagatctcgcgtg

SEQ ID NO: 47 - DNA, 807 bp, Artificial sequence
  nucleic acid encoding IRES-inhibitory peptide IP 1-01
ncnnnnntcngnctnaaaccacgctnggtaccgagctcggatccactagtaacggccgccagtgtgctggaattccaccatggacaggtcagactacaaggacgtcgacgacaaggcttatcaattgatgcagcgataccggtactagtcggaccgcatatgcccgggcgtaccgcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgcttttcttccctttcctttctcgccacggttcgcccggctttccccggtcaagnctctaaaatcggggggcctcccttttaangggtttcccgaatttaggtggcctttnacgggcaccctccgaacccccaaaaaaancttggatttaanggggtggaatgggtttcaaccggtaagntgngggcccc

SEQ ID NO: 48 - DNA, 858 bp, Artificial sequence
  nucleic acid encoding IRES-inhibitory peptide IP 1-02
gcnnnntncnnnccttaaaccactgcttggtaccgagctcggatccactagtaacggccgccagtgtgctggaataccaccatggacaggtcagactacaaggacgacgacgacaaggcttatcaatcaatcatagaggtaccaattgatgcatcgataccggtactagtcggaccgcatatgcccgggcgtaccgcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcnttctattctggggggtggggtggggcaggacagcaagggggaggattgggaagacaatagcaggcatgctggggatgcggtgggctctatggcttctgaggcggaaagaaccagctggggcttctagggggtatccccacncgccctgtagcggcgcattaancngcggcggggtgtggtgggttaaccncccaccgtgaccgctacannttgctcannngcccctaanccgccccgnatccctttttcgccntttcttncccntttaccctttncntccgcccaacgntttcgncccnngccttttcccccgnttcnaatttttcntatnattccggnnggggncttcncccnttttnagtgggntttcangaanctncannntgntctntttacccngnncaccntttgnangccccnnanaaaanannnnncnaanttactgcncntgc

SEQ ID NO: 49 - DNA, 138 bp, Artificial sequence
  nucleic acid encoding IRES-inhibitory peptide IP 1-04
aggtcagactacaaggacgacgacgacaaggtttatcaatcaatgcgccgatgcagggtaccaattgatgcatcgataccggtactagtcggaccgcatatgcccgggcgtaccgcggccgctcgagcatgcatctag

SEQ ID NO: 50 - DNA, 837 bp, Artificial sequence
  nucleic acid encoding IRES-inhibitory peptide IP 1-06
ncnnnntcngnnctnaaaccacgctnggtaccgagctcggatccactagtaacggccgccagtgtgctggaattccatggaattccaccatggacaggtcagactacaaggacgacgacgacaaggttgtggtgtctagaaaagggtaccaattgatgcatcgataccggtactagtcggaccgcatatgcccgggcgtaccgcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcatttaagcgcggcgggtgtggtggtttacnccgcagccgtgaccgctacacttngccagcggccctagccgcccgctccttttcggctttcttcccctttccttttctccgcccaccgttttcgcccgggcntttccccccgnttcaaaagncttcctaaaaattcngngggggcctccccctttttaannggnttttcccggaatttttagcttggcttttnancgggncaanccttncnaaaccccccaaaaaaanaactttggac

SEQ ID NO: 51 - DNA, 837 bp, Artificial sequence
  nucleic acid encoding IRES-inhibitory peptide IP 1-09
ncnnngncngnccttaaaccacttctaggtaccgagctcggatccactagtaacggccgccagtgtgctggaattccaccatggacaggtcagactacaaggacgacgacgacaaggtggtcgaccgcaagggtaccaattgatgcatcgataccggtactagtcggaccgcatatgcccgggcgtaccgcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccanctggggctctagggggtatccccacgcgccctgtagcggcgcattaagcgcggcgggtgtggtggttacgcgcagcgtgaccgctacactttgccagcgccctagcgcccgcctccttttcgctttccttcccttttcctttttctcgccaccgttttcggcccgggcttttcccccgttcaacgccttcttaaaaattcgggggggcttcccccttttagggggtttcccgnaatttttaaggttgccttttttnacgggncaaccctncgaaccccccaanaaaaaaacntttngaatntaagggggttgattggntttcc

SEQ ID NO: 52 - DNA, 825 bp, Artificial sequence
  nucleic acid encoding IRES-inhibitory peptide IP 1-17
ncnnngncgnncctttaaaccactnctnggtaccgagctcggatccactagtaacggccgccagtgtgctggaattccaccatggacaggtcagactacaaggacgacgacgacacaattgatgcatcgataccggtactagtcggaccgcatatgcccgggcgtaccgcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttccctttccttttctccgccacgtttcgccgggcttttcccccgtcaagctctaaatccggggggcttcccctttntaggggtttcccggattttaagtggctttttacnggcnaccctccgaaccccccaaaaaaaaacntttgaantttagggggttgantgggtttcnaccgtttagttgggggcccattccgccccctngaac

SEQ ID NO: 53 - DNA, 129 bp, Artificial sequence
  nucleic acid encoding IRES-inhibitory peptide IP 1-18
aggtcagactacaaggacgacgacgacaaggcttatcaatcaatcacggtaccaattgatgcatcgataccggtactagtcggaccgcatatgcccgggcgtaccgcggccgctcgagcatgcatctag

SEQ ID NO: 54 - DNA, 837 bp, Artificial sequence
  nucleic acid encoding IRES-inhibitory peptide IP 1-22
ncnnntnnntcngncctttaaaccacncttggtaccgagctcggatccactagtaacggccgccagtgtgctggaattccaccatggacaggtcagactacaaggacgacgacgacaaggcttatcaatcaatcacgtttgattgattcaattgatgcatcgataccggtactagtcggaccgcatatgcccgggcgtaccgcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacccgcagcgtgaccgctacacttgccagcgccctagccgcccggctcccttttcgccttttcttccctttccctttttctccgcccacggnttccgccngngcnttttcccccgnttcaaacnctccctaaaaattcgggggggggcctcccctttttanggggnttcccnnaattttaagttggcattttaaccgggccaacccttcngaaccccccnaaaaaaaaaactttggatnttnac

SEQ ID NO: 55 - DNA, 813 bp, Artificial sequence
  nucleic acid encoding IRES-inhibitory peptide IP 1-23
ncnnnnnnncngncttnaaaccactnctnggtaccgagctcggatccactagtaacggccgccagtgtgctggaattccaccatggacaggtcagactacaaggacgacgacgacaagtttaaccggtaccaattgatgcatcgataccggtactagtcggaccgcatatgcccgggcgtaccgcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagccgccctagcgcccgctcctttcgcctttcttcccttcctttctcgccacggttcggccggcctttccccgntcaaagcntctaaantcgggggggctccccttttangggttcccgaattttaagtngctttttacgggnaaccctncgnanccccaaaaaaaaacttttgaatttagngggtggaatgggttttcaacngntnatcc

SEQ ID NO: 56 - DNA, 825 bp, Artificial sequence
  nucleic acid encoding IRES-inhibitory peptide IP 2-01
ccnnnntnccggnctnnaaaaccactncttggtaccgagctcggatccactagtaacggccgccagtgtgctggaattccaccatggacaggtcagactacaaggacgacgacgacaaggcttatcaatcaatcaggggcgtgccgatgaaggtaccaattgatgcatcgataccggtactagtcggaccgcatatgcccgggcgtaccgcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcnggcatgctggggatgcggtgggctctatggcttctgaggcggaaagaaccagctggggctctagggggtatccccaacgcgccctgtancggcgcattaancncggcggngtgtggtggtttacgcgcagcgtgaccgctncncttncccagccgccctancgccccggcttccttttcgctttccttccctttccttttctcncccacggtttcgccgggccttttccccccgttcnaagctncttaaactcggnggggcctcccnttttnagggggcctcccnaattttaanctngccttttanccgggncanccttccngaanccccaaaaaataaancc

SEQ ID NO: 57 - DNA, 819 bp, Artificial sequence
  nucleic acid encoding IRES-inhibitory peptide IP 2-02
ncnnntncnnncttaaaccangcttggtaccgagctcggatccactagtaacggccgccagtgtgctggaattccaccatggacaggtcagactacaaggacgacgacgacaaggcttatcaatcaatcacgcgcctgatcaattgatgcatcgataccggtactagtcggaccgcatatgcccgggcgtaccgcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacactttgccagcgccctagcgcccgcctcctttcggctttctttcccctttcccttttctcgcccacgtttcgcccnggctttcccccgtcnaaagcctnctaaaaatcggggggngccttccccctttnaggggnttcccggaattttaagtngctttttaacgggcaaccctncgaaccccccaaaaaaaanctttggaatttnanngngttg

SEQ ID NO: 58 - DNA, 836 bp, Artificial sequence
  nucleic acid encoding IRES-inhibitory peptide IP 2-04
ncnnnnncggncctttaaaccactgcttggtaccgagctcggatccactagtaacggccgccagtgtgctggaattccaccatggacaggtagactacaaggacgacgacgacaaggcttatcaatcaatcagcgcctatgattgatcaattgatgcatcgataccggtactagtcggaccgcatatgcccgggcgtaccgcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcatttaancgcggcgggtgtggtggtttacncgcagcgtgaccgctncacttgccagcgccctaagccgccccgctccttttcgcttttctttccctttcccttttctccgccaccgtttcggcccgggcttttcccccgttcaaaggcttccttaaaatccggggggggccttccccctttttagggggtntccccgaanttntaagttggcnttttaacngggcacccnttcgaaaccccccaaaanaaaaactttggantttaaggn

SEQ ID NO: 59 - DNA, 853 bp, Artificial sequence
  nucleic acid encoding IRES-inhibitory peptide IP 2-09
ncnnngncggncctttaaaaccactnctagnctaccnancgcggatncnctactaacggncgccagtgtgctggaattccaccatggacaggtcagactacaaggacgacgacgacaaggcttatcaatcaatcattcgctgattgattgagtcttgtcgtagtcgtccaattgatgcatcgataccggtactagtcggaccgcatatgcccgggcgtaccgcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcnagggggaggattgggaanacaatancnggcatgctggggatgcggtgggctctatggcttctgaggcgganaagaaccagctggggctctagggggtatccccacgcncccctgtancggcgcattnaaaccnccgcgggtgtggtnggttaccncgcatccgctgaccngnntnacacttggcccagacgcccctnncctcccccgccnccntttatcgncttttcctncccactttccnttttnantcgncccccgnntanngcccgggccttttttcccccgtttnnaaggcctcttnaaaatncngggnggggcnnccccttttantagggtntcccnganttttnacntggcctttttaacgngnncaccccttcnnaccc

SEQ ID NO: 60 - DNA, 920 bp, Artificial sequence
  nucleic acid encoding IRES-inhibitory peptide IP 2-10
gcnnngnctggtnannngnaacncnnttaggctaccaanctcggatccnctantaacggncgccagngtgctggaattccaccatggacaggtcagactacaaggacgatgacgacaaggcttatctcaattgatgcatcgataccggtactagtcggaccgcatatgcccgggcgtaccgcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactggcctttcctaattaaatgaggaaattgcctcncattgntctgaatangtgtcattctnttctngggggtngggtggggganganaagccaaggggangantgggaaaaaacattaacnngccttgcttgggnnttccggtggggctcntnnnggtttccnnaggnngggaaaanaaanccaanntngggggncnnnnaaggggggtttntccccccacncccccccnnnnnaaccgnggccnttnaaacncccccgncgggtttttngtnngtntncncccnncnacnctntnnancccngntntnanactttnggcnaagngggccctntaaccgccccggtctccnttttcngcnnttcctttcccccttttccttttntctcggcccanngtttngcnccccggttttttccccnnggnanaaanncnncncaaaattttcgggggggcccccccnttntnaagggggtttccccnaattttnnngnggnttttnannggngnccccnccnaaccccccaaaaaaaattttnantttaagggngnnaangggntcccacttttatgtggggccccacc

SEQ ID NO: 61 - DNA, 63 bp, Artificial sequence
  nucleic acid encoding IRES-inhibitory peptide IP 1-01
aggtcagactacaaggacgtcgacgacaaggcttatcaattgatgcagcgataccggtactag

SEQ ID NO: 62 - DNA, 132 bp, Artificial sequence
  nucleic acid encoding IRES-inhibitory peptide IP 1-02
aggtcagactacaaggacgacgacgacaaggcttatcaatcaatcatagaggtaccaattgatgcatcgataccggtactagtcggaccgcatatgcccgggcgtaccgcggccgctcgagcatgcatctag

SEQ ID NO: 63 - DNA, 138 bp, Artificial sequence; identical to SEQ ID NO: 49
  nucleic acid encoding IRES-inhibitory peptide IP 1-04

SEQ ID NO: 64 - DNA, 81 bp, Artificial sequence
  nucleic acid encoding IRES-inhibitory peptide IP 1-06
aggtcagactacaaggacgacgacgacaaggttgtggtgtctagaaaagggtaccaattgatgcatcgataccggtactag

SEQ ID NO: 65 - DNA, 57 bp, Artificial sequence
  nucleic acid encoding IRES-inhibitory peptide IP 1-09
aggtcagactacaaggacgacgacgacaaggtggtcgaccgcaagggtaccaattga

SEQ ID NO: 66 - DNA, 105 bp, Artificial sequence
  nucleic acid encoding IRES-inhibitory peptide IP 1-17
aggtcagactacaaggacgacgacgacacaattgatgcatcgataccggtactagtcggaccgcatatgcccgggcgtaccgcggccgctcgagcatgcatctag

SEQ ID NO: 67 - DNA, 129 bp, Artificial sequence; identical to SEQ ID NO: 53
  nucleic acid encoding IRES-inhibitory peptide IP 1-18

SEQ ID NO: 68 - DNA, 57 bp, Artificial sequence
  nucleic acid encoding IRES-inhibitory peptide IP 1-22
aggtcagactacaaggacgacgacgacaaggcttatcaatcaatcacgtttgattga

SEQ ID NO: 69 - DNA, 69 bp, Artificial sequence
  nucleic acid encoding IRES-inhibitory peptide IP 1-23
aggtcagactacaaggacgacgacgacaagtttaaccggtaccaattgatgcatcgataccggtactag

SEQ ID NO: 70 - DNA, 144 bp, Artificial sequence
  nucleic acid encoding IRES-inhibitory peptide IP 2-01
aggtcagactacaaggacgacgacgacaaggcttatcaatcaatcaggggcgtgccgatgaaggtaccaattgatgcatcgataccggtactagtcggaccgcatatgcccgggcgtaccgcggccgctcgagcatgcatctag

SEQ ID NO: 71 - DNA, 63 bp, Artificial sequence
  nucleic acid encoding IRES-inhibitory peptide IP 2-02
aggtcagactacaaggacgacgacgacaaggcttatcaatcaatcacgcgcctgatcaattga

SEQ ID NO: 72 - DNA, 54 bp, Artificial sequence
  nucleic acid encoding IRES-inhibitory peptide IP 2-04
gactacaaggacgacgacgacaaggcttatcaatcaatcagcgcctatgattga

SEQ ID NO: 73 - DNA, 54 bp, Artificial sequence
  nucleic acid encoding IRES-inhibitory peptide IP 2-09
aggtcagactacaaggacgacgacgacaaggcttatcaatcaatcattcgctga

SEQ ID NO: 74 - DNA, 45 bp, Artificial sequence
  nucleic acid encoding IRES-inhibitory peptide IP 2-10
aggtcagactacaaggacgatgacgacaaggcttatctcaattga

SEQ ID NO: 75 - protein, 20 aa, Artificial sequence
  IRES-inhibitory peptide IP 1-01 with FLAG tag
RSDYKDVDDKAYQLMQRYRY

SEQ ID NO: 76 - protein, 43 aa, Artificial sequence
  IRES-inhibitory peptide IP 1-02 with FLAG tag
RSDYKDDDDKAYQSIIEVPIDASIPVLVGPHMPGRTAAARACI

SEQ ID NO: 77 - protein, 45 aa, Artificial sequence
  IRES-inhibitory peptide IP 1-04 with FLAG tag
RSDYKDDDDKVYQSMRRCRVPIDASIPVLVGPHMPGRTAAARACI

SEQ ID NO: 78 - protein, 26 aa, Artificial sequence
  IRES-inhibitory peptide IP 1-06 with FLAG tag
RSDYKDDDDKVVVSRKGYQLMHRYRY

SEQ ID NO: 79 - protein, 18 aa, Artificial sequence
  IRES-inhibitory peptide IP 1-09 with FLAG tag
RSDYKDDDDKVVDRKGTN

SEQ ID NO: 80 - protein, 34 aa, Artificial sequence
  IRES-inhibitory peptide IP 1-17 with FLAG tag
RSDYKDDDDTIDASIPVLVGPHMPGRTAAARACI

SEQ ID NO: 81 - protein, 42 aa, Artificial sequence
  IRES-inhibitory peptide IP 1-18 with FLAG tag
RSDYKDDDDKAYQSITVPIDASIPVLVGPHMPGRTAAARACI

SEQ ID NO: 82 - protein, 18 aa, Artificial sequence
  IRES-inhibitory peptide IP 1-22 with FLAG tag
RSDYKDDDDKAYQSITFD

SEQ ID NO: 83 - protein, 22 aa, Artificial sequence
  IRES-inhibitory peptide IP 1-23 with FLAG tag
RSDYKDDDDKFNRYQLMHRYRY

SEQ ID NO: 84 - protein, 47 aa, Artificial sequence
  IRES-inhibitory peptide IP 2-01 with FLAG tag
RSDYKDDDDKAYQSIRGVPMKVPIDASIPVLVGPHMPGRTAAARACI

SEQ ID NO: 85 - protein, 20 aa, Artificial sequence
  IRES-inhibitory peptide IP 2-02 with FLAG tag
RSDYKDDDDKAYQSITRLIN

SEQ ID NO: 86 - protein, 17 aa, Artificial sequence
  IRES-inhibitory peptide IP 2-04 with FLAG tag
DYKDDDDKAYQSISAYD

SEQ ID NO: 87 - protein, 17 aa, Artificial sequence
  IRES-inhibitory peptide IP 2-09 with FLAG tag
RSDYKDDDDKAYQSIIR

SEQ ID NO: 88 - protein, 14 aa, Artificial sequence
  IRES-inhibitory peptide IP 2-10 with FLAG tag
RSDYKDDDDKAYLN

SEQ ID NO: 89 - protein, 10 aa, Artificial sequence
  IRES-inhibitory peptide IP 1-01
AYQLMQRYRY

SEQ ID NO: 90 - protein, 33 aa, Artificial sequence
  IRES-inhibitory peptide IP 1-02
AYQSIIEVPIDASIPVLVGPHMPGRTAAARACI

SEQ ID NO: 91 - protein, 35 aa, Artificial sequence
  IRES-inhibitory peptide IP 1-04
VYQSMRRCRVPIDASIPVLVGPHMPGRTAAARACI

SEQ ID NO: 92 - protein, 16 aa, Artificial sequence
  IRES-inhibitory peptide IP 1-06
VVVSRKGYQLMHRYRY

SEQ ID NO: 93 - protein, 8 aa, Artificial sequence
  IRES-inhibitory peptide IP 1-09
VVDRKGTN

SEQ ID NO: 94 - protein, 25 aa, Artificial sequence
  IRES-inhibitory peptide IP 1-17
TIDASIPVLVGPHMPGRTAAARACI

SEQ ID NO: 95 - protein, 32 aa, Artificial sequence
  IRES-inhibitory peptide IP 1-18
AYQSITVPIDASIPVLVGPHMPGRTAAARACI

SEQ ID NO: 96 - protein, 8 aa, Artificial sequence
  IRES-inhibitory peptide IP 1-22
AYQSITFD

SEQ ID NO: 97 - protein, 12 aa, Artificial sequence
  IRES-inhibitory peptide IP 1-23
FNRYQLMHRYRY

SEQ ID NO: 98 - protein, 37 aa, Artificial sequence
  IRES-inhibitory peptide IP 2-01
AYQSIRGVPMKVPIDASIPVLVGPHMPGRTAAARACI

SEQ ID NO: 99 - protein, 10 aa, Artificial sequence
  IRES-inhibitory peptide IP 2-02
AYQSITRLIN

SEQ ID NO: 100 - protein, 9 aa, Artificial sequence
  IRES-inhibitory peptide IP 2-04
AYQSISAYD

SEQ ID NO: 101 - protein, 7 aa, Artificial sequence
  IRES-inhibitory peptide IP 2-09
AYQSIIR

SEQ ID NO: 102 - protein, 4 aa, Artificial sequence
  IRES-inhibitory peptide IP 2-10
AYLN

SEQ ID NO: 103 - protein, 17 aa, Artificial sequence
  IRES-inhibitory peptide
IDASIPVLVGPHMPGRT

SEQ ID NO: 104 - protein, 24 aa, Artificial sequence
  IRES-inhibitory peptide
IDASIPVLVGPHMPGRTAAARACI

SEQ ID NO: 105 - protein, 19 aa, Artificial sequence
  IRES-inhibitory peptide
VPIDASIPVLVGPHMPGRT

SEQ ID NO: 106 - protein, 26 aa, Artificial sequence
  IRES-inhibitory peptide
VPIDASIPVLVGPHMPGRTAAARACI

SEQ ID NO: 107 - protein, 9 aa, Artificial sequence
  IRES-inhibitory peptide
YQLMQRYRY

SEQ ID NO: 108 - protein, 28 aa, Artificial sequence
  IRES-inhibitory peptide and HIV-1 TAT protein transduction domain
GRKKRRQRRRGIDASIPVLVGPHMPGRT

SEQ ID NO: 109 - protein, 30 aa, Artificial sequence
  IRES-inhibitory peptide and HIV-1 TAT protein transduction domain
GRKKRRQRRRGVPIDASIPVLVGPHMPGRT

SEQ ID NO: 110 - protein, 20 aa, Artificial sequence
  IRES-inhibitory peptide and HIV-1 TAT protein transduction domain
GRKKRRQRRRGYQLMQRYRY

SEQ ID NO: 111 - DNA, 33 bp, artificial sequence
  nucleic aicd encoding IRES inhibitory peptide IP 1-01 without FLAG sequence
gcttatcaattgatgcagcgataccggtactag

SEQ ID NO: 112 - DNA, 102 bp, artificial sequence
  nucleic aicd encoding IRES inhibitory peptide IP 1-02 without FLAG sequence
gcttatcaatcaatcatagaggtaccaattgatgcatcgataccggtactagtcggaccgcatatgcccgggcgtaccgcggccgctcgagcatgcatctag

SEQ ID NO: 113 - DNA, 108 bp, artificial sequence
  nucleic aicd encoding IRES inhibitory peptide IP 1-04 without FLAG sequence
gtttatcaatcaatgcgccgatgcagggtaccaattgatgcatcgataccggtactagtcggaccgcatatgcccgggcgtaccgcggccgctcgagcatgcatctag

SEQ ID NO: 114 - DNA, 51 bp, artificial sequence
  nucleic aicd encoding IRES inhibitory peptide IP 1-06 without FLAG sequence
gttgtggtgtctagaaaagggtaccaattgatgcatcgataccggtactag

SEQ ID NO: 115 - DNA, 27 bp, artificial sequence
  nucleic aicd encoding IRES inhibitory peptide IP 1-09 without FLAG sequence
gtggtcgaccgcaagggtaccaattga

SEQ ID NO: 116 - DNA, 81 bp, artificial sequence
  nucleic aicd encoding IRES inhibitory peptide IP 1-17 without FLAG sequence
gacacaattgatgcatcgataccggtactagtcggaccgcatatgcccgggcgtaccgcggccgctcgagcatgcatctag

SEQ ID NO: 117 - DNA, 99 bp, artificial sequence
  nucleic aicd encoding IRES inhibitory peptide IP 1-18 without FLAG sequence
gcttatcaatcaatcacggtaccaattgatgcatcgataccggtactagtcggaccgcatatgcccgggcgtaccgcggccgctcgagcatgcatctag

SEQ ID NO: 118 - DNA, 27 bp, artificial sequence
  nucleic aicd encoding IRES inhibitory peptide IP 1-22 without FLAG sequence
gcttatcaatcaatcacgtttgattga

SEQ ID NO: 119 - DNA, 39 bp, artificial sequence
  nucleic aicd encoding IRES inhibitory peptide IP 1-23 without FLAG sequence
tttaaccggtaccaattgatgcatcgataccggtactag

SEQ ID NO: 120 - DNA, 114 bp, artificial sequence
  nucleic aicd encoding IRES inhibitory peptide IP 2-01 without FLAG sequence
gcttatcaatcaatcaggggcgtgccgatgaaggtaccaattgatgcatcgataccggtactagtcggaccgcatatgcccgggcgtaccgcggccgctcgagcatgcatctag

SEQ ID NO: 121 - DNA, 33 bp, artificial sequence
  nucleic aicd encoding IRES inhibitory peptide IP 2-02 without FLAG sequence
gcttatcaatcaatcacgcgcctgatcaattga

SEQ ID NO: 122 - DNA, 30 bp, artificial sequence
  nucleic aicd encoding IRES inhibitory peptide IP 2-04 without FLAG sequence
gcttatcaatcaatcagcgcctatgattga

SEQ ID NO: 123 - DNA, 24 bp, artificial sequence
  nucleic aicd encoding IRES inhibitory peptide IP 2-09 without FLAG sequence
gcttatcaatcaatcattcgctga

SEQ ID NO: 124 - DNA, 15 bp, artificial sequence
  nucleic aicd encoding IRES inhibitory peptide IP 2-10 without FLAG sequence
gcttatctcaattga

SEQ ID NO: 125 - DNA, 54 bp, artificial sequence
  nucleic aicd encoding IRES inhibitory peptide
ccaattgatgcatcgataccggtactagtcggaccgcatatgcccgggcgtacc

SEQ ID NO: 126 - DNA, 54 bp, artificial sequence; identical to SEQ ID NO: 125
  nucleic aicd encoding IRES inhibitory peptide

SEQ ID NO: 127 - DNA, 51 bp, artificial sequence
  nucleic aicd encoding IRES inhibitory peptide
attgatgcatcgataccggtactagtcggaccgcatatgcccgggcgtacc

SEQ ID NO: 128 - DNA, 54 bp, artificial sequence; identical to SEQ ID NO: 125
  nucleic aicd encoding IRES inhibitory peptide

SEQ ID NO: 129 - DNA, 54 bp, artificial sequence; identical to SEQ ID NO: 125
  nucleic aicd encoding IRES inhibitory peptide

SEQ ID NO: 130 - DNA, 57 bp, artificial sequence
  nucleic aicd encoding IRES inhibitory peptide
gtaccaattgatgcatcgataccggtactagtcggaccgcatatgcccgggcgtacc

SEQ ID NO: 131 - DNA, 57 bp, artificial sequence; identical to SEQ ID NO: 130
  nucleic aicd encoding IRES inhibitory peptide

SEQ ID NO: 132 - DNA, 57 bp, artificial sequence; identical to SEQ ID NO: 130
  nucleic aicd encoding IRES inhibitory peptide

SEQ ID NO: 133 - DNA, 57 bp, artificial sequence; identical to SEQ ID NO: 130
  nucleic aicd encoding IRES inhibitory peptide

SEQ ID NO: 134 - DNA, 27 bp, artificial sequence
  nucleic aicd encoding IRES inhibitory peptide
tatcaattgatgcagcgataccggtac

SEQ ID NO: 135 - DNA, 27 bp, artificial sequence
  nucleic aicd encoding IRES inhibitory peptide
taccaattgatgcatcgataccggtac

SEQ ID NO: 136 - DNA, 27 bp, artificial sequence
  nucleic aicd encoding IRES inhibitory peptide
taccaattgatgcatcgataccggtac

SEQ ID NO: 137 - DNA, 87 bp, artificial sequence
  nucleic aicd encoding IRES inhibitory peptide with TAT protein transduction domain
ggtcgtaaaaaacgtcgtcaacgtcgtcgtggtccaattgatgcatcgataccggtactagtcggaccgcatatgcccgggcgtacc

SEQ ID NO: 138 - DNA, 87 bp, artificial sequence; identical to SEQ ID NO: 137
  nucleic aicd encoding IRES inhibitory peptide with TAT protein transduction domain

SEQ ID NO: 139 - DNA, 84 bp, artificial sequence
  nucleic aicd encoding IRES inhibitory peptide with TAT protein transduction domain
ggtcgtaaaaaacgtcgtcaacgtcgtcgtggtattgatgcatcgataccggtactagtcggaccgcatatgcccgggcgtacc

SEQ ID NO: 140 - DNA, 87 bp, artificial sequence; identical to SEQ ID NO: 137
  nucleic aicd encoding IRES inhibitory peptide with TAT protein transduction domain

SEQ ID NO: 141 - DNA, 87 bp, artificial sequence; identical to SEQ ID NO: 137
  nucleic aicd encoding IRES inhibitory peptide with TAT protein transduction domain

SEQ ID NO: 142 - DNA, 90 bp, artificial sequence
  nucleic aicd encoding IRES inhibitory peptide with TAT protein transduction domain
ggtcgtaaaaaacgtcgtcaacgtcgtcgtggtgtaccaattgatgcatcgataccggtactagtcggaccgcatatgcccgggcgtacc

SEQ ID NO: 143 - DNA, 90 bp, artificial sequence; identical to SEQ ID NO: 142
  nucleic aicd encoding IRES inhibitory peptide with TAT protein transduction domain

SEQ ID NO: 144 - DNA, 90 bp, artificial sequence; identical to SEQ ID NO: 142
  nucleic aicd encoding IRES inhibitory peptide with TAT protein transduction domain

SEQ ID NO: 145 - DNA, 90 bp, artificial sequence; identical to SEQ ID NO: 142
  nucleic aicd encoding IRES inhibitory peptide with TAT protein transduction domain

SEQ ID NO: 146 - DNA, 63 bp, artificial sequence
  nucleic aicd encoding IRES inhibitory peptide with TAT protein transduction domain
ggtcgtaaaaaacgtcgtcaacgtcgtcgtggtgtatatcaattgatgcagcgataccggtac

SEQ ID NO: 147 - DNA, 63 bp, artificial sequence
  nucleic aicd encoding IRES inhibitory peptide with TAT protein transduction domain
ggtcgtaaaaaacgtcgtcaacgtcgtcgtggtgtataccaattgatgcatcgataccggtac

SEQ ID NO: 148 - DNA, 63 bp, artificial sequence; identical to SEQ ID NO: 147
  nucleic aicd encoding IRES inhibitory peptide with TAT protein transduction domain